(19) United States
(12) Patent Application Publication
(10) Pub. No.: US 2016/0178584 A1
(43) Pub. Date: Jun. 23, 2016

(54) METHODS FOR DIAGNOSING AND ASSESSING KIDNEY DISEASE

(71) Applicant: THE REGENTS OF THE UNIVERSITY OF CALIFORNIA, Oakland, CA (US)

(72) Inventors: Kumar SHARMA, Del Mar, CA (US); Robert NAVIAUX, San Diego, CA (US)

(21) Appl. No.: 14/963,138

(22) Filed: Dec. 8, 2015

Related U.S. Application Data
(63) Continuation of application No. 13/843,680, filed on Mar. 15, 2013, now abandoned.
Application No. 13/843,680 is a continuation-in-part of application No. PCT/US2011/056229, filed on Oct. 13, 2011.
(60) Provisional application No. 61/393,276, filed on Oct. 14, 2010.
(60) Provisional application No. 61/670,985, filed on Jul. 12, 2012.

(51) Int. Cl. G01N 30/72; G01N 30/00; G01N 33/487

(52) U.S. Cl. CPC G01N 30/7206; G01N 33/487; G01N 30/00; G01N 2800/347; G01N 2800/56

(57) ABSTRACT

The technology relates in part to methods for identifying the presence of kidney disease, determining the level of kidney disease, or the progression of kidney disease, in a subject that has or has not been diagnosed with diabetes. The technology further relates to methods for determining the targets for therapy for kidney disease, the efficacy of a treatment for kidney disease, and methods for determining the toxicity of a therapeutic in a subject with kidney disease.

The technology relates in part to methods for identifying the presence of kidney disease, determining the level of kidney disease, or the progression of kidney disease, in a subject that has or has not been diagnosed with diabetes. The technology further relates to methods for determining the targets for therapy for kidney disease, the efficacy of a treatment for kidney disease, and methods for determining the toxicity of a therapeutic in a subject with kidney disease.

### RELATED APPLICATIONS

This application is a continuation of U.S. patent application Ser. No. 13/843,680, filed Mar. 15, 2013, now pending, which is a continuation-in-part application (“CIP”) of Patent Convention Treaty (PCT) International Application Serial No: PCT/US2011/056229, filed Oct. 13, 2011, which claims benefit of priority to U.S. Provisional Patent Application Ser. No. 61/393,276, filed Oct. 14, 2010. U.S. patent application Ser. No. 13/843,680, filed Mar. 15, 2013, now pending, also claims the benefit of priority under 35 U.S.C. §119(e) of U.S. Provisional Patent Application Ser. No. 61/670,985, filed Jul. 12, 2012. The aforementioned applications are expressly incorporated herein by reference in their entirety and for all purposes.

### GOVERNMENT RIGHTS

This invention was made with government support under grants DK063017, and NIDDK grant number 7R01DK063017-05/1DP3DK094352-01, all awarded by the National Institutes of Health. The government has certain rights in the invention.

### TECHNICAL FIELD

This invention pertains to the fields of medical therapeutics and diagnostics. In alternative embodiments, the technology relates in part to methods for identifying the presence of kidney disease, determining the level of kidney disease, or the progression of kidney disease, in a subject that has or has not been diagnosed with diabetes. The technology further relates to methods for determining the targets for therapy for kidney disease, the efficacy of a treatment for kidney disease, and methods for determining the toxicity of a therapeutic in a subject with kidney disease.

The technology relates in part to methods for identifying the presence of kidney disease, determining the level of kidney disease, or the progression of kidney disease, in a subject that has or has not been diagnosed with diabetes. The technology further relates to methods for determining the targets for therapy for kidney disease, the efficacy of a treatment for kidney disease, and methods for determining the toxicity of a therapeutic in a subject with kidney disease.

### BACKGROUND

Diabetes is the leading cause of chronic kidney disease in the United States, but the disease can be caused by other diseases and disorders such as, for example, cardiovascular disease, hypertension, and obesity. Often, there are no symptoms of the disease until the kidneys are seriously and irreversibly damaged. Therefore, it is important to have tools to diagnose early stages of the disease.

Current markers for kidney disease are blood creatinine and urine protein. The blood creatinine is thought to reflect the filtration rate of the nephron, but may be in the normal range, despite significant disease, and may not indicate progression or improvement. The urine protein may indicate leakage of blood protein into the urine and may indicate kidney disease in some patients, but not all, and may also not indicate whether the disease is improving or deteriorating.

There is a need for methods of determining the likelihood or level of kidney disease in a patient. There is also a need for methods for determining the effectiveness or toxicity of a kidney disease therapy in a patient. Further, there is a need for method for identifying individual molecules or metabolites that have been found to distinguish patients with diabetic kidney disease from normal, non-diabetic subjects, or patients with diabetic kidney disease from patients with diabetes and no kidney disease, kidney disease of non-diabetic causes from diabetic kidney disease, and patients with diabetes and no kidney disease from normal, non-diabetic subjects.

### SUMMARY

The invention provides and relates in part to methods for identifying the presence of kidney disease, determining the level of kidney disease, or the progression of kidney disease, in a subject that has or has not been diagnosed with diabetes. The technology further relates to methods for determining the efficacy of a treatment for kidney disease, and methods for determining the toxicity of a therapeutic in a subject with kidney disease, or toxicity to the kidneys. The technology further relates to the identification of biomarkers that indicate kidney disease, for example, diabetic kidney disease. The biomarkers reflect mitochondrial function and the overall health of the organ. The technology may, for example, also be used to identify a therapeutic.

A method is provided of identifying the presence or level of kidney disease in a subject, including determining the level of at least one organic acid of glycolic acid, 3-OH isobutyric acid, 3-OH isovaleric acid, aconitic acid, homovanillic acid, citric acid, uracil, fumaric acid, oleic acid, or azelaic acid in a subject. A comparison is provided of the level of the at least one organic acid to a reference level of the at least one organic acid from a control or the same subject collected at a previous time. Detection can be either simultaneous or sequential and may be from the same biological sample or from multiple samples from the same or different subjects. The identification of the presence of kidney disease in the subject where the at least one organic acid level in the subject is decreased when compared to the reference level of the at least one organic acid. It is appreciated that at least one organic acid may include any number up to all ten organic acids and combination of said acids alone or in combination with other small organic molecules with a molecular weight of less than 700 Daltons or metabolites such as, for example, blood creatinine or blood urea nitrogen.

Independently, or in combination with the determination of the at least one organic acid, the level of 5-oxoproline in a subject is determined. By comparing the level of 5-oxoproline to a reference level of 5-oxoproline, the presence or level of kidney disease in the subject where the level of 5-oxoproline in the subject is increased when compared to the reference 5-oxoproline level is identified. Other diseases so identified include diabetes or diabetic kidney disease.

It is appreciated that the reference level of the organic acid or the 5-oxoproline is readily determined from a healthy patient or from a sample obtained from the subject at an earlier time. In other aspects, the reference level of the organic acid or the 5-oxoproline is determined from an analysis of samples obtained from more than one healthy patient.

The level of the at least one organic acid is decreased at least 1.5-, 2-, 3-, 4-fold or even lower compared to the reference level. The level of 5-oxoproline is increased at least 1.5-, 2-, 3-, 4-fold or even lower compared to the reference level.

In other aspects, in the methods of the embodiments, the subject has not been diagnosed with diabetes. In other aspects, the subject has kidney disease. In other aspects, the level of the organic acid or the 5-oxoproline is determined using gas chromatography. In other aspects, the level of the organic acid or the 5-oxoproline is determined using mass spectrometry. In other aspects, the level of the organic acid is determined from a biological sample from the subject. In other aspects, the sample contains urine, or a urine fraction, or blood or a blood fraction. Although certain examples of detection of the level of an organic acid are presented herein, the technology is not limited to these examples, as various methods of detection of organic acid levels may be used.

In some embodiments, a method is provided of determining the progression of kidney disease over time in a subject diagnosed with kidney disease, comprising determining the level of at least one organic acid of glycolic acid, 3-OH isobutyric acid, 3-OH isovaleric acid, aconitic acid, homovanillic acid, citric acid, uracil, fumaric acid, oleic acid, or azelaic acid in a subject; comparing the level of the at least one organic acid to the level of the at least one organic acid determined in a sample obtained from the subject at an earlier time point; and determining that the kidney disease has progressed in the subject where the at least one organic acid level in the subject is decreased when compared to the level determined in the sample obtained from the subject at the earlier time point. It is appreciated that the at least one organic acid is two such acids, three such acids, four such acids, five such acids, six such acids, 7 such, 8 such, 9 such or all 10 of such acids, alone or in combination with other small organic molecules with a molecular weight of less than 700 Daltons or metabolites such as creatinine.

In other aspects of any of the methods of the embodiment, the method further comprises determining the level of 5-oxoproline in a subject, comparing the level to the level determined in a sample obtained from the subject at an earlier time point, and determining that kidney disease has progressed in the subject where the level of 5-oxoproline in the subject is increased when compared to the 5-oxoproline level in the sample obtained from the subject at the earlier time point.

In some aspects, the subject has diabetes. In other aspects, the subject has diabetic kidney disease. In other aspects, the level of the organic acid or acids is decreased at least 1.5 fold compared to the level in the sample obtained from the subject at the earlier time point. In other aspects, the level of the organic acid or acids is decreased at least 2 fold compared to the level in the sample obtained from the subject at the earlier time point. In other aspects, the level of 5-oxoproline is increased at least 3 fold compared to the level in the sample obtained from the subject at the earlier time point. In other aspects, the level of 5-oxoproline is increased at least 4 fold compared to the level in the sample obtained from the subject at the earlier time point. In other aspects, the subject has not been diagnosed with diabetes. In other aspects, the subject has kidney disease. In other aspects, the level of the organic acid or the 5-oxoproline is determined using gas chromatography. In other aspects, the level of the organic acid or the 5-oxoproline is determined using mass spectrometry. In other aspects, the level of the organic acid is determined from a biological sample from the subject. In other aspects, the sample contains urine or a urine fraction.

In some embodiments, a method is provided, comprising: administering a therapeutic to a subject diagnosed with kidney disease; determining the level of at least one organic acid selected from the group consisting of glycolic acid, 3-OH isobutyric acid, 3-OH isovaleric acid, aconitic acid, homovanillic acid, citric acid, uracil, fumaric acid, oleic acid, or azelaic acid in the subject; and determining whether the dosage of the therapeutic subsequently administered to the subject is adjusted based on the level of the at least one organic acid.

In some embodiments, a method is provided, comprising determining the level of at least one organic acid selected from the group consisting of glycolic acid, 3-OH isobutyric acid, 3-OH isovaleric acid, aconitic acid, homovanillic acid, citric acid, uracil, fumaric acid, oleic acid, or azelaic acid in a subject diagnosed with kidney disease, wherein the subject has been administered a therapeutic; and maintaining a subsequent dosage of the therapeutic or adjusting a subsequent dosage of the therapeutic administered to the subject based on the level of the at least one organic acid in the subject.

In some embodiments, a method is provided comprising determining the level of at least one organic acid selected from the group consisting of glycolic acid, 3-OH isobutyric acid, 3-OH isovaleric acid, aconitic acid, homovanillic acid, citric acid, uracil, fumaric acid, oleic acid, or azelaic acid in a subject diagnosed with kidney disease, wherein the subject has been administered a therapeutic; and determining whether the dosage of the therapeutic subsequently administered to the subject is adjusted based on the level of the at least one organic acid in the subject.

In some embodiments, a method is provided comprising receiving information comprising the level of at least one organic acid selected from the group consisting of glycolic acid, 3-OH isobutyric acid, 3-OH isovaleric acid, aconitic acid, homovanillic acid, citric acid, uracil, fumaric acid, oleic acid, or azelaic acid in a subject to whom a therapeutic has been administered; and maintaining a subsequent dosage of the therapeutic, or adjusting a subsequent dosage of the therapeutic to the subject based on the level of the at least one organic acid in the subject.

In some embodiments, a method is provided comprising determining the level of at least one organic acid selected from the group consisting of glycolic acid, 3-OH isobutyric acid, 3-OH isovaleric acid, aconitic acid, homovanillic acid, citric acid, uracil, fumaric acid, oleic acid, or azelaic acid in a subject diagnosed with kidney disease, wherein the subject has been administered a therapeutic; and transmitting the determined level to a decision maker who maintains a subsequent dosage of the therapeutic or adjusts a subsequent dosage of the therapeutic to the subject based on the level of the at least one organic acid identified in the subject.

In some embodiments, a method is provided comprising determining the level of at least one organic acid selected from the group consisting of glycolic acid, 3-OH isobutyric acid, 3-OH isovaleric acid, aconitic acid, homovanillic acid, citric acid, uracil, fumaric acid, oleic acid, or azelaic acid in a subject diagnosed with kidney disease, wherein the subject has been administered a therapeutic; and transmitting an indication to maintain a subsequent dosage of the therapeutic or adjust a subsequent dosage of the therapeutic to the subject based on the level of the at least one organic acid in the subject.

In some embodiments, a method is provided comprising administering a therapeutic to a subject diagnosed with kidney disease; determining the level of at least one organic acid selected from the group consisting of glycolic acid, 3-OH isobutyric acid, 3-OH isovaleric acid, aconitic acid, homovanillic acid, citric acid, uracil, fumaric acid, oleic acid, or azelaic acid in the subject; maintaining a subsequent dosage of the therapeutic, or adjusting a subsequent dosage of the therapeutic to the subject based on the level of the at least one organic acid in the subject.

In some aspects, the method comprises determining the levels of at least two organic acids selected from the group consisting of glycolic acid, 3-OH isobutyric acid, 3-OH isovaleric acid, aconitic acid, homovanillic acid, citric acid, uracil, fumaric acid, oleic acid, or azelaic acid. In some aspects, the method comprises determining the levels of at least three organic acids selected from the group consisting of glycolic acid, 3-OH isobutyric acid, 3-OH isovaleric acid, aconitic acid, homovanillic acid, citric acid, uracil, fumaric acid, oleic acid, or azelaic acid. In some aspects, the method comprises determining the levels of at least four organic acids selected from the group consisting of glycolic acid, 3-OH isobutyric acid, 3-OH isovaleric acid, aconitic acid, homovanillic acid, citric acid, uracil, fumaric acid, oleic acid, or azelaic acid. In some aspects, the method comprises determining the levels of at least five organic acids selected from the group consisting of glycolic acid, 3-OH isobutyric acid, 3-OH isovaleric acid, aconitic acid, homovanillic acid, citric acid, uracil, fumaric acid, oleic acid, or azelaic acid. In some aspects, the method comprises determining the levels of at least six organic acids selected from the group consisting of glycolic acid, 3-OH isobutyric acid, 3-OH isovaleric acid, aconitic acid, homovanillic acid, citric acid, uracil, fumaric acid, oleic acid, or azelaic acid. In some aspects, the method comprises determining the levels of at least seven organic acids selected from the group consisting of glycolic acid, 3-OH isobutyric acid, 3-OH isovaleric acid, aconitic acid, homovanillic acid, citric acid, uracil, fumaric acid, oleic acid, or azelaic acid. In some aspects, the method comprises determining the levels of at least eight organic acids selected from the group consisting of glycolic acid, 3-OH isobutyric acid, 3-OH isovaleric acid, aconitic acid, homovanillic acid, citric acid, uracil, fumaric acid, oleic acid, or azelaic acid. In some aspects, the method comprises determining the levels of at least nine organic acids selected from the group consisting of glycolic acid, 3-OH isobutyric acid, 3-OH isovaleric acid, aconitic acid, homovanillic acid, citric acid, uracil, fumaric acid, oleic acid, or azelaic acid. In some aspects, the subject has diabetes. In some aspects, the subject has not been diagnosed with diabetes. In some aspects, the level of at least one organic acid is determined using gas chromatography. In some aspects, the level of the organic acid is determined using mass spectrometry. In some aspects, the level of the organic acid is determined from a biological sample from the subject. In some aspects, the sample contains urine or a urine fraction.

In some embodiments, a method is provided for reducing toxicity of a treatment, comprising; administering a therapeutic to a subject in need thereof; determining the level of at least one organic acid selected from the group consisting of glycolic acid, 3-OH isobutyric acid, 3-OH isovaleric acid, aconitic acid, homovanillic acid, citric acid, uracil, fumaric acid, oleic acid, or azelaic acid in the subject; and maintaining a subsequent dosage of the therapeutic or adjusting a subsequent dosage of the therapeutic to the subject based on the level of the at least one organic acid in the subject.

In some aspects, the method comprises determining the levels of at least two organic acids selected from the group consisting of glycolic acid, 3-OH isobutyric acid, 3-OH isovaleric acid, aconitic acid, homovanillic acid, citric acid, uracil, fumaric acid, oleic acid, or azelaic acid. In some aspects, the method comprises determining the levels of at least three organic acids selected from the group consisting of glycolic acid, 3-OH isobutyric acid, 3-OH isovaleric acid, aconitic acid, homovanillic acid, citric acid, uracil, fumaric acid, oleic acid, or azelaic acid. In some aspects, the method comprises determining the levels of at least four organic acids selected from the group consisting of glycolic acid, 3-OH isobutyric acid, 3-OH isovaleric acid, aconitic acid, homovanillic acid, citric acid, uracil, fumaric acid, oleic acid, or azelaic acid. In some aspects, the method comprises determining the levels of at least five organic acids selected from the group consisting of glycolic acid, 3-OH isobutyric acid, 3-OH isovaleric acid, aconitic acid, homovanillic acid, citric acid, uracil, fumaric acid, oleic acid, or azelaic acid. In some aspects, the method comprises determining the levels of at least six organic acids selected from the group consisting of glycolic acid, 3-OH isobutyric acid, 3-OH isovaleric acid, aconitic acid, homovanillic acid, citric acid, uracil, fumaric acid, oleic acid, or azelaic acid. In some aspects, the method comprises determining the levels of at least seven organic acids selected from the group consisting of glycolic acid, 3-OH isobutyric acid, 3-OH isovaleric acid, aconitic acid, homovanillic acid, citric acid, uracil, fumaric acid, oleic acid, or azelaic acid. In some aspects, the method comprises determining the levels of at least eight organic acids selected from the group consisting of glycolic acid, 3-OH isobutyric acid, 3-OH isovaleric acid, aconitic acid, homovanillic acid, citric acid, uracil, fumaric acid, oleic acid, or azelaic acid. In some aspects, the method comprises determining the levels of at least nine organic acids selected from the group consisting of glycolic acid, 3-OH isobutyric acid, 3-OH isovaleric acid, aconitic acid, homovanillic acid, citric acid, uracil, fumaric acid, oleic acid, or azelaic acid. In some aspects, the method comprises determining the level of the at least one organic acid in the subject before administering the therapeutic, and comparing the level in the subject before administering the therapeutic to the level in the subject after administering the therapeutic. In some aspects, the level of the organic acid is determined from a biological sample from the subject. In some aspects, the sample contains urine or a urine fraction. In some aspects, the level of the organic acid is determined using mass spectrometry. In some aspects, the level of the organic acid is determined from a biological sample from the subject.

In some embodiments, methods are provided for identifying the presence or level of kidney disease in a subject, comprising determining the level of at least one organic acid selected from the group consisting of glycolic acid, 3-OH isobutyric acid, 3-OH isovaleric acid, aconitic acid, homovanillic acid, citric acid, uracil, fumaric acid, oleic acid and azelaic acid in a sample obtained from the subject; comparing the level of the at least one organic acid with a reference level of the at least one organic acid, wherein the reference level has been determined from at least one sample collected from the same subject at a different time period; or the reference level has been determined from a sample or samples collected from one or more other subjects; and identifying the presence or level of kidney disease in the subject where the at least one organic acid level in the subject is decreased when compared to the reference level of the at least one organic acid. In some aspects, the level at least two organic acids selected from the group consisting of glycolic acid, 3-OH isobutyric acid, 3-OH isovaleric acid, aconitic acid, homovanillic acid, citric acid, uracil, fumaric acid, oleic acid, and azelaic acid, are determined, compared to at least two reference organic acids, and the presence or level of kidney disease in the subject is identified where the at least two organic acid levels in the subject are decreased when compared to the at least two reference organic acid levels.

In some aspects, the level of at least three organic acids selected from the group consisting of glycolic acid, 3-OH isobutyric acid, 3-OH isovaleric acid, aconitic acid, homovanillic acid, citric acid, uracil, fumaric acid, oleic acid, and azelaic acid, are determined, compared to at least three reference organic acids, and the presence or level of kidney disease in the subject is identified where the at least three organic acid levels in the subject are decreased when compared to the at least three reference organic acid levels. In some aspects, the level of at least four organic acids selected from the group consisting of glycolic acid, 3-OH isobutyric acid, 3-OH isovaleric acid, aconitic acid, homovanillic acid, citric acid, uracil, fumaric acid, oleic acid, and azelaic acid, are determined, compared to at least four reference organic acids, and the presence or level of kidney disease in the subject is identified where the at least four organic acid levels in the subject are decreased when compared to the at least four reference organic acid levels. In some aspects, the level of at least five organic acids selected from the group consisting of glycolic acid, 3-OH isobutyric acid, 3-OH isovaleric acid, aconitic acid, homovanillic acid, citric acid, uracil, fumaric acid, oleic acid, and azelaic acid, are determined, compared to at least five reference organic acids, and the presence or level of kidney disease in the subject is identified where the at least five organic acid levels in the subject are decreased when compared to the at least five reference organic acid levels. In some aspects, the level of at least six organic acids selected from the group consisting of glycolic acid, 3-OH isobutyric acid, 3-OH isovaleric acid, aconitic acid, homovanillic acid, citric acid, uracil, fumaric acid, oleic acid, and azelaic acid, are determined, compared to at least six reference organic acids, and the presence or level of kidney disease in the subject is identified where the at least six organic acid levels in the subject are decreased when compared to the at least six reference organic acid levels. In some aspects, the level of at least seven organic acids selected from the group consisting of glycolic acid, 3-OH isobutyric acid, 3-OH isovaleric acid, aconitic acid, homovanillic acid, citric acid, uracil, fumaric acid, oleic acid, and azelaic acid, are determined, compared to at least seven reference organic acids, and the presence or level of kidney disease in the subject is identified where the at least seven organic acid levels in the subject are decreased when compared to the at least seven reference organic acid levels. In some aspects, the level of at least eight organic acids selected from the group consisting of glycolic acid, 3-OH isobutyric acid, 3-OH isovaleric acid, aconitic acid, homovanillic acid, citric acid, uracil, fumaric acid, oleic acid, and azelaic acid, are determined, compared to at least eight reference organic acids, and the presence or level of kidney disease in the subject is identified where the at least eight organic acid levels in the subject are decreased when compared to the at least eight reference organic acid levels. In some aspects, the level of at least nine organic acids selected from the group consisting of glycolic acid, 3-OH isobutyric acid, 3-OH isovaleric acid, aconitic acid, homovanillic acid, citric acid, uracil, fumaric acid, oleic acid, and azelaic acid, are determined, compared to at least nine reference organic acids, and the presence or level of kidney disease in the subject is identified where the at least nine organic acid levels in the subject are decreased when compared to the at least nine reference organic acid levels. In some aspects, the level of at least ten organic acids selected from the group consisting of glycolic acid, 3-OH isobutyric acid, 3-OH isovaleric acid, aconitic acid, homovanillic acid, citric acid, uracil, fumaric acid, oleic acid, and azelaic acid, are determined, compared to at least ten reference organic acids, and the presence or level of kidney disease in the subject is identified where the at least ten organic acid levels in the subject are decreased when compared to the at least ten reference organic acid levels.

In some embodiments, the method further comprises determining the level of 5-oxoproline in a sample obtained from the subject, comparing the level to the level to a reference level of 5-oxoproline, and identifying the presence or level of kidney disease in the subject where the level of 5-oxoproline in the subject is increased when compared to the reference 5-oxoproline level. In some embodiments, the method also comprises determining the level of citrate in a sample obtained from the subject, comparing the level to the level to a reference level of citrate, and identifying the presence or level of kidney disease in the subject where the level of citrate in the subject is decreased when compared to the reference citrate level.

In some embodiments, the level of the organic acid or acids is decreased at least 1.5 fold compared to the reference level. In some embodiments, the level of the organic acid or acids is decreased at least 2 fold compared to the reference level. In some embodiments, the level of 5-oxoproline is increased compared to the reference level. In some embodiments, the level of citrate is increased compared to the reference level.

Also provided are methods of determining the progression of kidney disease over time in a subject diagnosed with kidney disease, comprising determining the level of at least one organic acid selected from the group consisting of glycolic acid, 3-OH isobutyric acid, 3-OH isovaleric acid, aconitic acid, homovanillic acid, citric acid, uracil, fumaric acid, oleic acid and azelaic acid, in a sample obtained from the subject; comparing the level of the at least one organic acid to the level of the at least one organic acid determined in a sample obtained from the subject at an earlier time point; determining that the kidney disease has progressed in the subject where the at least one organic acid level in the subject is decreased when compared to the level determined in the sample obtained from the subject at the earlier time point. In some embodiments, the method comprises determining the level of at least two organic acids selected from the group consisting of glycolic acid, 3-OH isobutyric acid, 3-OH isovaleric acid, aconitic acid, homovanillic acid, citric acid, uracil, fumaric acid, oleic acid and azelaic acid, comparing the level of the at least two organic acids to the level of the at least two organic acids determined in a sample obtained from the subject at an earlier time point, and determining that the kidney disease has progressed in the subject where the at least two organic acid levels in the subject are decreased when compared to the levels determined in the sample obtained from the subject at the earlier time point. In some embodiments, the method comprises determining the level of at least three, four, five, six, seven, eight, nine, or ten organic acids selected from the group consisting of glycolic acid, 3-OH isobutyric acid, 3-OH isovaleric acid, aconitic acid, homovanillic acid, citric acid, uracil, fumaric acid, oleic acid and azelaic acid, comparing the level of the at least two organic acids to the level of the at least three, four, five, six, seven, eight, nine, or ten organic acids, respectively, determined in a sample obtained from the subject at an earlier time point, and determining that the kidney disease has progressed in the subject where the at least three, four, five, six, seven, eight, nine, or ten, respectively, organic acid levels in the subject are decreased when compared to the levels determined in the sample obtained from the subject at the earlier time point. In some embodiments, the method further comprises determining the level of 5-oxoproline in a sample obtained from the subject, comparing the level to the level determined in a sample obtained from the subject at an earlier time point, and determining that kidney disease has progressed in the subject where the level of 5-oxoproline in the subject is increased when compared to the 5-oxoproline level in the sample obtained from the subject at the earlier time point. In some embodiments, the method further comprises determining the level of citrate in a sample obtained from the subject, comparing the level to the level determined in a sample obtained from the subject at an earlier time point, and determining that kidney disease has progressed in the subject where the level of citrate in the subject is increased when compared to the citrate level in the sample obtained from the subject at the earlier time point.

In some embodiments, the level of the organic acid or acids is decreased at least 1.5 fold compared to the level in the sample obtained from the subject at the earlier time point. In some embodiments, the level of the organic acid or acids is decreased at least 2 fold compared to the level in the sample obtained from the subject at the earlier time point.

In certain embodiments, the subject has diabetes. In certain embodiments, the subject has diabetic kidney disease. In certain embodiments, the subject has not been diagnosed with diabetes. In certain embodiments, the subject has kidney disease.

In some embodiments, the level of the organic acid, the 5-oxoproline, or the citrate is determined using gas chromatography. In some embodiments, the level of the organic acid, the 5-oxoproline, or the citrate is determined using mass spectrometry. In some embodiments, the level of the organic acid is determined from a biological sample from the subject. In some embodiments, the sample contains urine or a urine fraction, or blood or a blood fraction. Also provided are methods comprising administering a therapeutic to a subject diagnosed with kidney disease; determining the level of at least one organic acid selected from the group consisting of glycolic acid, 3-OH isobutyric acid, 3-OH isovaleric acid, aconitic acid, homovanillic acid, citric acid, uracil, fumaric acid, oleic acid and azelaic acid, in a sample obtained from the subject; and determining whether the dosage of the therapeutic subsequently administered to the subject is adjusted based on the level of the at least one organic acid. In some embodiments, a method is provided comprising determining the level of at least one organic acid selected from the group consisting of glycolic acid, 3-OH isobutyric acid, 3-OH isovaleric acid, aconitic acid, homovanillic acid, citric acid, uracil, fumaric acid, oleic acid and azelaic acid, in a sample obtained from a subject diagnosed with kidney disease, wherein the subject has been administered a therapeutic; and maintaining a subsequent dosage of the therapeutic or adjusting a subsequent dosage of the therapeutic administered to the subject based on the level of the at least one organic acid in the sample. In some embodiments the method comprises determining the level of at least two, three, four, five, six, seven, eight, nine, or ten organic acids selected from the group consisting of glycolic acid, 3-OH isobutyric acid, 3-OH isovaleric acid, aconitic acid, homovanillic acid, citric acid, uracil, fumaric acid, oleic acid and azelaic acid, and maintaining a subsequent dosage of the therapeutic or adjusting a subsequent dosage of the therapeutic administered to the subject based on the levels of at least two, three, four, five, six, seven, eight, nine, or ten, respectively, organic acids in the sample. In some embodiments, the method further comprises determining the level of 5-oxoproline in a sample obtained from the subject, and maintaining a subsequent dosage of the therapeutic or adjusting a subsequent dosage of the therapeutic administered to the subject based on the levels of 5-oxoproline in the sample. In some embodiments, the method further comprises determining the level of citrate in a sample obtained from the subject, and maintaining a subsequent dosage of the therapeutic or adjusting a subsequent dosage of the therapeutic administered to the subject based on the levels of citrate in the sample.

Also provided in some embodiments are methods for reducing toxicity of a treatment, comprising: determining the pre-treatment level of at least one organic acid selected from the group consisting of glycolic acid, 3-OH isobutyric acid, 3-OH isovaleric acid, aconitic acid, homovanillic acid, citric acid, uracil, fumaric acid, oleic acid and azelaic acid, in a sample obtained from the subject; administering a therapeutic to the subject; determining the post-treatment level of the at least one organic acid selected from the group consisting of glycolic acid, 3-OH isobutyric acid, 3-OH isovaleric acid, aconitic acid, homovanillic acid, citric acid, uracil, fumaric acid, oleic acid and azelaic acid, in the subject after; and lowering the subsequent dosage of the therapeutic where the post-treatment level of the at least one organic acid is decreased compared to the pre-treatment level of the at least one organic acid in the sample. In some embodiments, the method comprises determining the levels of at least two, three, four, five, six, seven, eight, nine, or ten organic acids selected from the group consisting of glycolic acid, 3-OH isobutyric acid, 3-OH isovaleric acid, aconitic acid, homovanillic acid, citric acid, uracil, fumaric acid, oleic acid and azelaic acid, and lowering the subsequent dosage of the therapeutic where the post-treatment levels of the at least two, three, four, five, six, seven, eight, nine, or ten organic acids are decreased compared to the pre-treatment levels of the at least two, three, four, five, six, seven, eight, nine, or ten, respectively, organic acids in the sample. In some embodiments, the method further comprises determining the pre-treatment level of 5-oxoproline, in a sample obtained from the subject, determining the post-treatment level of 5-oxoproline, in the subject, and lowering the subsequent dosage of the therapeutic where the post-treatment level of 5-oxoproline is increased compared to the pre-treatment level of 5-oxoproline in the sample. In some embodiments, the method further comprises the pre-treatment level of citrate, in the subject, determining the post-treatment level of citrate, in a sample obtained from the subject, and lowering the subsequent dosage of the therapeutic where the post-treatment level of citrate is decreased compared to the pre-treatment level of citrate in the sample.

Also provided in some embodiments are methods of identifying the presence or level of diabetes related complications in a subject, comprising determining the level of at least one metabolite selected from the group consisting of lactic acid, glycolic acid, fumaric acid, malic acid, adipic acid, 2-OH-glutaric acid, aconitic acid, homovanillic acid, stearic acid, 3-OH-isobutyric acid, palmitic acid, and citrate in a sample obtained from the subject; comparing the level of the at least one metabolite with a reference level of the at least one metabolite, wherein the reference level has been determined from at least one sample collected from the same subject at a different time period; or the reference level has been determined from a sample or samples collected from one or more other subjects; and identifying the presence or level of diabetes-related complications in the subject where the at least one metabolite level in the subject is decreased when compared to the reference level of the at least one metabolite. In some embodiments, the level of at least two, three, four, five, six, seven, eight, nine, ten, eleven, or twelve metabolites are determined, compared to at least two, three, four, five, six, seven, eight, nine, ten, eleven, or twelve, reference metabolites, and the presence or level of diabetes related complications in the subject is identified where the at least two, three, four, five, six, seven, eight, nine, ten, eleven, or twelve, respectively, metabolite levels in the subject are decreased when compared to the at least two, three, four, five, six, seven, eight, nine, ten, eleven, or twelve, reference metabolite levels. In some embodiments, the diabetes related complication is a microvascular complication. In some embodiments, the diabetes related complication is a macrovascular complication.

Also provided in some embodiments are methods of determining the progression of a diabetes related complication over time in a subject diagnosed with a diabetes related complication, comprising determining the level at least one metabolite selected from the group consisting of lactic acid, glycolic acid, fumaric acid, malic acid, adipic acid, 2-OH-glutaric acid, aconitic acid, homovanillic acid, stearic acid, 3-OH-isobutyric acid, palmitic acid, and citrate in a sample obtained from the subject; comparing the level of the at least one metabolite to the level of the at least one metabolite determined in a sample obtained from the subject at an earlier time point; and determining that the diabetes related complication has progressed in the subject where the at least one metabolite level in the subject is decreased when compared to the level determined in the sample obtained from the subject at the earlier time point. In some embodiments, the method comprises determining the progression of a diabetes related complication over time in a subject diagnosed with a diabetes related complication, comprising determining the level at least two, three, four, five, six, seven, eight, nine, ten, eleven, or twelve metabolites selected from the group consisting of lactic acid, glycolic acid, fumaric acid, malic acid, adipic acid, 2-OH-glutaric acid, aconitic acid, homovanillic acid, stearic acid, 3-OH-isobutyric acid, palmitic acid, and citrate in a sample obtained from the subject; comparing the level of the at least two, three, four, five, six, seven, eight, nine, ten, eleven, or twelve metabolites to the level of the at least two, three, four, five, six, seven, eight, nine, ten, eleven, or twelve metabolites, respectively determined in a sample obtained from the subject at an earlier time point; and determining that the diabetes related complication has progressed in the subject where the at least two, three, four, five, six, seven, eight, nine, ten, eleven, or twelve metabolite levels in the subject are decreased when compared to the levels determined in the sample obtained from the subject at the earlier time point.

Also provided in some embodiments are methods comprising: administering a therapeutic to a subject diagnosed with a diabetes related complication; determining the level of at least one metabolite selected from the group consisting of lactic acid, glycolic acid, fumaric acid, malic acid, adipic acid, 2-OH-glutaric acid, aconitic acid, homovanillic acid, stearic acid, 3-OH-isobutyric acid, palmitic acid, and citrate in a sample obtained from the subject; and determining whether the dosage of the therapeutic subsequently administered to the subject is adjusted based on the level of the at least one metabolite. In some embodiments, the method comprises determining the level of at least two, three, four, five, six, seven, eight, nine, ten, eleven, or twelve metabolites selected from the group consisting of lactic acid, glycolic acid, fumaric acid, malic acid, adipic acid, 2-OH-glutaric acid, aconitic acid, homovanillic acid, stearic acid, 3-OH-isobutyric acid, palmitic acid, and citrate in a sample obtained from the subject; and determining whether the dosage of the therapeutic subsequently administered to the subject is adjusted based on the level of the at least two, three, four, five, six, seven, eight, nine, ten, eleven, or twelve metabolites, respectively.

Also provided in some embodiments are methods of identifying the presence or level of diabetes, cardiovascular disease, hypertension, or chronic kidney disease in an obese subject, comprising determining the level of at least one metabolite selected from the group consisting of lactic acid, glycolic acid, fumaric acid, malic acid, adipic acid, 2-OH-glutaric acid, aconitic acid, homovanillic acid, stearic acid, 3-OH-isobutyric acid, palmitic acid, and citrate in a sample obtained from the subject; comparing the level of the at least one metabolite with a reference level of the at least one metabolite, wherein the reference level has been determined from at least one sample collected from the same subject at a different time period; or the reference level has been determined from a sample or samples collected from one or more other subjects; and identifying the presence or level of diabetes-related complications in the subject where the at least one metabolite level in the subject is decreased when compared to the reference level of the at least one metabolite. In some embodiments, the method comprises determining the level of at least two, three, four, five, six, seven, eight, nine, ten, eleven, or twelve metabolites selected from the group consisting of lactic acid, glycolic acid, fumaric acid, malic acid, adipic acid, 2-OH-glutaric acid, aconitic acid, homovanillic acid, stearic acid, 3-OH-isobutyric acid, palmitic acid, and citrate in a sample obtained from the subject; comparing the level of the at least two, three, four, five, six, seven, eight, nine, ten, eleven, or twelve metabolites with a reference level of the at least two, three, four, five, six, seven, eight, nine, ten, eleven, or twelve metabolites, wherein the reference level has been determined from at least one sample collected from the same subject at a different time period; or the reference level has been determined from a sample or samples collected from one or more other subjects; and identifying the presence or level of diabetes-related complications in the subject where the at least two, three, four, five, six, seven, eight, nine, ten, eleven, or twelve metabolite levels in the subject is decreased when compared to the reference levels of the at least two, three, four, five, six, seven, eight, nine, ten, eleven, or twelve metabolites, respectively.

Also provided in some embodiments are methods of identifying the presence or level of diabetes, cardiovascular disease, hypertension, or chronic kidney disease in an obese subject, comprising determining the level of at least one metabolite selected from the group consisting of lactic acid, glycolic acid, fumaric acid, malic acid, adipic acid, 2-OH-glutaric acid, aconitic acid, homovanillic acid, stearic acid, 3-OH-isobutyric acid, palmitic acid, and citrate in a sample obtained from the subject; comparing the level of the at least one metabolite with a reference level of the at least one metabolite, wherein the reference level has been determined from at least one sample collected from the same subject at a different time period; or the reference level has been determined from a sample or samples collected from one or more other subjects; and identifying the presence or level of diabetes, cardiovascular disease, hypertension, or chronic kidney disease in the subject where the at least one metabolite level in the subject is decreased when compared to the reference level of the at least one metabolite. In some embodiments, the method comprises determining the level of at least two, three, four, five, six, seven, eight, nine, ten, eleven, or twelve metabolites selected from the group consisting of lactic acid, glycolic acid, fumaric acid, malic acid, adipic acid, 2-OH-glutaric acid, aconitic acid, homovanillic acid, stearic acid, 3-OH-isobutyric acid, palmitic acid, and citrate in a sample obtained from the subject; comparing the level of the at least two, three, four, five, six, seven, eight, nine, ten, eleven, or twelve metabolites with a reference level of the at least two, three, four, five, six, seven, eight, nine, ten, eleven, or twelve metabolites, wherein the reference level has been determined from at least one sample collected from the same subject at a different time period; or the reference level has been determined from a sample or samples collected from one or more other subjects; and identifying the presence or level of diabetes, cardiovascular disease, hypertension, or chronic kidney disease in the subject where the at least two, three, four, five, six, seven, eight, nine, ten, eleven, or twelve metabolites level in the subject is decreased when compared to the reference level of the at least two, three, four, five, six, seven, eight, nine, ten, eleven, or twelve metabolites, respectively.

Also provided in some embodiments are methods of identifying the presence or level of hypertension in a subject, comprising determining the level of at least one metabolite selected from the group consisting of lactic acid, glycolic acid, fumaric acid, malic acid, adipic acid, 2-OH-glutaric acid, aconitic acid, homovanillic acid, stearic acid, 3-OH-isobutyric acid, palmitic acid, and citric acid in a sample obtained from the subject; comparing the level of the at least one metabolite with a reference level of the at least one metabolite, wherein the reference level has been determined from at least one sample collected from the same subject at a different time period; or the reference level has been determined from a sample or samples collected from one or more other subjects; and identifying the presence or level of hypertension in the subject where the at least one metabolite level in the subject is decreased when compared to the reference level of the at least one metabolite. In some embodiments, the method comprises determining the level of at least two, three, four, five, six, seven, eight, nine, ten, eleven, or twelve metabolites selected from the group consisting of lactic acid, glycolic acid, fumaric acid, malic acid, adipic acid, 2-OH-glutaric acid, aconitic acid, homovanillic acid, stearic acid, 3-OH-isobutyric acid, palmitic acid, and citric acid in a sample obtained from the subject; comparing the level of the at least two, three, four, five, six, seven, eight, nine, ten, eleven, or twelve metabolites with a reference level of the at least two, three, four, five, six, seven, eight, nine, ten, eleven, or twelve metabolites, wherein the reference level has been determined from at least one sample collected from the same subject at a different time period; or the reference level has been determined from a sample or samples collected from one or more other subjects; and identifying the presence or level of hypertension in the subject where the at least two, three, four, five, six, seven, eight, nine, ten, eleven, or twelve metabolite levels in the subject is decreased when compared to the reference level of the at least two, three, four, five, six, seven, eight, nine, ten, eleven, or twelve metabolites, respectively.

Also provided in some embodiments are methods of identifying the presence or level of liver disease in a subject having obesity, diabetes, or chronic kidney disease, comprising determining the level of at least one metabolite selected from the group consisting of lactic acid, glycolic acid, fumaric acid, malic acid, adipic acid, 2-OH-glutaric acid, aconitic acid, homovanillic acid, stearic acid, 3-OH-isobutyric acid, palmitic acid, and citric acid in a sample obtained from the subject; comparing the level of the at least one metabolite with a reference level of the at least one metabolite, wherein the reference level has been determined from at least one sample collected from the same subject at a different time period; or the reference level has been determined from a sample or samples collected from one or more other subjects; and identifying the presence or level of liver disease in the subject where the at least one metabolite level in the subject is decreased when compared to the reference level of the at least one metabolite. In some embodiments, the method comprises determining the level of at least two, three, four, five, six, seven, eight, nine, ten, eleven, or twelve metabolites selected from the group consisting of lactic acid, glycolic acid, fumaric acid, malic acid, adipic acid, 2-OH-glutaric acid, aconitic acid, homovanillic acid, stearic acid, 3-OH-isobutyric acid, palmitic acid, and citric acid in a sample obtained from the subject; comparing the level of the at least two, three, four, five, six, seven, eight, nine, ten, eleven, or twelve metabolites with a reference level of the at least two, three, four, five, six, seven, eight, nine, ten, eleven, or twelve metabolites, wherein the reference level has been determined from at least one sample collected from the same subject at a different time period; or the reference level has been determined from a sample or samples collected from one or more other subjects; and identifying the presence or level of liver disease in the subject where the at least two, three, four, five, six, seven, eight, nine, ten, eleven, or twelve metabolites level in the subject is decreased when compared to the reference level of the at least two, three, four, five, six, seven, eight, nine, ten, eleven, or twelve metabolites, respectively.

Also provided in some embodiments are methods of identifying the presence or level of joint involvement in a subject having obesity, diabetes, or chronic kidney disease, comprising determining the level of at least one metabolite selected from the group consisting of lactic acid, glycolic acid, fumaric acid, malic acid, adipic acid, 2-OH-glutaric acid, aconitic acid, homovanillic acid, stearic acid, 3-OH-isobutyric acid, palmitic acid, citric acid, and 5-oxoproline in a sample obtained from the subject; comparing the level of the at least one metabolite with a reference level of the at least one metabolite, wherein the reference level has been determined from at least one sample collected from the same subject at a different time period; or the reference level has been determined from a sample or samples collected from one or more other subjects; and identifying the presence or level of joint involvement in the subject where the at least one metabolite level in the subject is decreased when compared to the reference level of the at least one metabolite. In some embodiments, the method comprises determining the level of at least two, three, four, five, six, seven, eight, nine, ten, eleven, twelve, or thirteen metabolites selected from the group consisting of lactic acid, glycolic acid, fumaric acid, malic acid, adipic acid, 2-OH-glutaric acid, aconitic acid, homovanillic acid, stearic acid, 3-OH-isobutyric acid, palmitic acid, citric acid, and 5-oxoproline in a sample obtained from the subject; comparing the level of the at least two, three, four, five, six, seven, eight, nine, ten, eleven, twelve, or thirteen metabolites with a reference level of the at least one metabolite, wherein the reference level has been determined from at least one sample collected from the same subject at a different time period; or the reference level has been determined from a sample or samples collected from one or more other subjects; and identifying the presence or level of joint involvement in the subject where the at least two, three, four, five, six, seven, eight, nine, ten, eleven, twelve or thirteen metabolites level in the subject is decreased when compared to the reference level of the at least two, three, four, five, six, seven, eight, nine, ten, eleven, twelve, or thirteen metabolites, respectively.

Also provided in some embodiments are methods of identifying the presence or level of sleep apnea, restrictive lung disease or obstructive lung disease in a subject having diabetes, obesity, or chronic kidney disease, comprising determining the level of at least one metabolite selected from the group consisting of lactic acid, glycolic acid, fumaric acid, malic acid, adipic acid, 2-OH-glutaric acid, aconitic acid, homovanillic acid, stearic acid, 3-OH-isobutyric acid, palmitic acid, citric acid, and 5-oxoproline in a sample obtained from the subject; comparing the level of the at least one metabolite with a reference level of the at least one metabolite, wherein the reference level has been determined from at least one sample collected from the same subject at a different time period; or the reference level has been determined from a sample or samples collected from one or more other subjects; and identifying the presence or level of sleep apnea, restrictive lung disease or obstructive lung disease in the subject where the at least one metabolite level in the subject is decreased when compared to the reference level of the at least one metabolite.

In some embodiments, the method comprises determining the level of at least two, three, four, five, six, seven, eight, nine, ten, eleven, or twelve metabolites selected from the group consisting of lactic acid, glycolic acid, fumaric acid, malic acid, adipic acid, 2-OH-glutaric acid, aconitic acid, homovanillic acid, stearic acid, 3-OH-isobutyric acid, palmitic acid, citric acid, and 5-oxoproline in a sample obtained from the subject; comparing the level of the at least two, three, four, five, six, seven, eight, nine, ten, eleven, or twelve metabolites with a reference level of the at least one metabolite, wherein the reference level has been determined from at least one sample collected from the same subject at a different time period; or the reference level has been determined from a sample or samples collected from one or more other subjects; and identifying the presence or level of sleep apnea, restrictive lung disease or obstructive lung disease in the subject where the at least two, three, four, five, six, seven, eight, nine, ten, eleven, or twelve metabolites level in the subject is decreased when compared to the reference level of the at least two, three, four, five, six, seven, eight, nine, ten, eleven, or twelve metabolites, respectively.

In certain embodiments, the subject has diabetes. In certain embodiments, the subject has diabetic kidney disease. In certain embodiments, the subject has not been diagnosed with diabetes. In certain embodiments, the subject has kidney disease.

In some embodiments, the reference level of the organic acid, the metabolite, the 5-oxoproline, or the citrate is determined from a sample obtained from a healthy patient. In some embodiments, the reference level of the organic acid, the metabolite, the 5-oxoproline, or the citrate is determined from a sample obtained from the subject at an earlier time. In some embodiments, the reference level of the organic acid, the 5-oxoproline, or the citrate is determined from an analysis of samples obtained from more than one healthy patient. In certain embodiments, the organic acid is selected from the group consisting of glycolic acid, 3-OH isobutyric acid, 3-OH isovaleric acid, aconitic acid, homovanillic acid, citric acid, and uracil.

In some embodiments, the level of the organic acid, the metabolite, the 5-oxoproline, or the citrate is determined using gas chromatography. In some embodiments, the level of the organic acid, the metabolite, the 5-oxoproline, or the citrate is determined using mass spectrometry. In some embodiments, the level of the organic acid, the 5-oxoproline, the citrate, or the metabolite, is determined from a biological sample from the subject. In some embodiments, the sample contains urine, or a urine fraction, or blood or a blood fraction.

The technology relates in part to methods for identifying the presence of kidney disease, determining the level of kidney disease, or the progression of kidney disease, in a subject that has or has not been diagnosed with diabetes. The technology further relates to methods for determining the efficacy of a treatment for kidney disease, and methods for determining the toxicity of a therapeutic in a subject with kidney disease, or toxicity to the kidneys. The technology further relates to the identification of biomarkers that indicate kidney disease, for example, diabetic kidney disease. The biomarkers reflect mitochondrial function and the overall health of the organ. The technology may, for example, also be used to identify a therapeutic.

Thus, provided in certain embodiments are methods for identifying the presence or level of kidney disease in a subject, comprising determining the level of at least one, two, three, for, five, or six metabolites selected from the group consisting of 3-methyl adipic acid, 2-methyl acetoacetate, 3-methyl crotonyl glycine, 3-hydroxy propionate, 2-ethyl 3-OH propionate, and tiglylglycine in a sample obtained from the subject; comparing the level of the at least one metabolite with a reference level of the at least one metabolite, wherein the reference level has been determined from at least one sample collected from the same subject at a different time period; or the reference level has been determined from a sample or samples collected from one or more other subjects; and identifying the presence or level of kidney disease in the subject where the at least one metabolite level in the subject is decreased when compared to the reference level of the at least one metabolite. The methods may further comprise determining the level of at least one, two, three, four, five, six, seven, eight, nine, ten, eleven, or twelve additional metabolites selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate′ e, and tiglylglycine in a sample obtained from the subject, comparing the level of the at least one additional metabolite with a reference level of the at least one additional metabolite, wherein the reference level has been determined from at least one sample collected from the same subject at a different time period; or the reference level has been determined from a sample or samples collected from one or more other subjects; and identifying the presence or level of kidney disease in the subject where the at least one metabolite and the at least one additional metabolite levels in the subject are decreased when compared to the reference levels of the at least one metabolite and the at least one additional metabolite.

In some embodiments, the subject is human. In some embodiments, the subject has diabetes. In some embodiments, the subject has diabetic kidney disease. In some embodiments, the reference level of the metabolite, the 5-oxoproline, or the citrate is determined from a sample obtained from a healthy patient. In some embodiments, the reference level of the metabolite is determined from a sample obtained from the subject at an earlier time. In some embodiments, the reference level of the metabolite is determined from an analysis of samples obtained from more than one healthy patient. In some embodiments, the level of the metabolite or acids is decreased at least 1.5 fold compared to the reference level. In some embodiments, the level of the metabolite or acids is decreased at least 2 fold compared to the reference level. In some embodiments, the level of the metabolite or acids is decreased at least 3 fold compared to the reference level. In some embodiments, the level of the metabolite or acids is decreased at least 4 fold compared to the reference level. In some embodiments, the subject has not been diagnosed with diabetes. In some embodiments, the subject has kidney disease. In some embodiments, the level of the metabolite is determined using gas chromatography. In some embodiments, the level of the metabolite is determined using mass spectrometry. In some embodiments, the level of the metabolite is determined from a biological sample from the subject. In some embodiments, the sample contains urine, or a urine fraction, or blood, or a blood fraction.

In some embodiments, the levels of thirteen metabolites selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine are decreased when compared to the reference levels of the thirteen metabolites.

Also provided are methods comprising obtaining a sample from a subject; detecting the amount of a panel of metabolites comprising glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine and comparing said amount to the amount of the panel of metabolites in a sample obtained from the subject at an earlier time point. The method may further comprise providing an outcome based on the comparison. The outcome may be, for example, a determination that the level of at least one, two, three, four, five, six, seven, eight, nine, ten, eleven, twelve, or thirteen metabolites is decreased, a diagnosis, such as, for example, a diagnosis of diabetic kidney disease, a determination of toxicity of a therapeutic, the effectiveness of a therapeutic, or a determination that the level of a disease, such as diabetic kidney disease, has progressed. In some embodiments, the subject is human. In some embodiments, the subject has diabetes. In some embodiments, the subject has diabetic kidney disease. In some embodiments, the subject is obese.

Certain embodiments are described further in the following description, examples, claims, and drawings.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1 is a bar graph of citrate levels in patients with diabetic nephropathy who had been enrolled in a clinical trial and in normal volunteers. The patients had samples of urine evaluated prior to administration of an experimental drug (P) (n=49) or an FDA approved drug (Z) (n=24). For the purpose of this submission, patients were not treated before their urines were evaluated. As patients were entered into a clinical trial they met the inclusion and exclusion criteria of the trial and therefore well characterized. Urine metabolomics was performed on each individual sample. A cut-off p value of P<0.00846 was chosen to have a false detection rate less than 0.05 to account for multiple testing.

FIG. 2 is a series of bar graphs of data in control patients (n=23), patients having diabetes without chronic kidney disease (Diabetic; n=27), and patients having chronic kidney disease without diabetes (CKD, n=15), and patients having chronic kidney disease and diabetes (CKD-DM; n=27).

FIG. 3 is a series of bar graphs of data in control patients (n=23), patients having diabetes without chronic kidney disease (Diabetic; n=27), and patients having chronic kidney disease without diabetes (CKD, n=15), and patients having chronic kidney disease and diabetes (CKD-DM; n=27).

FIG. 4 is a series of bar graphs of data in control patients (n=23), patients having diabetes without chronic kidney disease (Diabetic; n=27), and patients having chronic kidney disease without diabetes (CKD, n=15), and patients having chronic kidney disease and diabetes (CKD-DM; n=27).

FIG. 5 graphically illustrates results of principal components analysis for metabolites altered in diabetic kidney disease. The figure shows the plot of principal component 1 (x-axis) versus principal component 2 (y-axis). Blue diamonds represent the control group, red squares represent the screening group, green triangles represent the validation group, purple circles represent the Type 1 diabetes group, and orange circles represent the type 2 diabetes group.

FIGS. 6A-H are a series of schematics illustrating a biochemical network map of urine metabolites altered in diabetic kidney disease. FIGS. 6A-C and E-H illustrate in detail insets A, B, C, D, E, F and G of FIG. 6D, as indicated below. Pink small circle nodes represent chemicals which are measured, and large red hexagonal nodes represent metabolites which are altered in diabetic renal disease. Grey nodes represent compounds which are not measured and grey rectangles represent enzymes. Compounds whose concentrations are significantly altered are shown in the magnified inserts, falling in the following areas of metabolism:

FIG. 6A illustrates in detailed area (magnified insert) D, of FIG. 6D, schematically illustrating valine catabolism (3-hydroxyisobutyric acid [HIBA]) and the isoleucine catabolism L-pathway (tiglylglycine [TigGly], 2-methylacetoacetic acid [2MAcAc]) and R-pathway (2-ethyl-3-hydroxypropionate [2E3HPropionate]).

FIG. 6B illustrates in detailed area (magnified insert) C, of FIG. 6D, schematically illustrating leucine catabolism (3-hydroxyisovaleric acid [3HIVA] and valine catabolism (3-hydroxyisobutyric acid [HIBA]).

FIG. 6C illustrates in detailed area (magnified insert) E, of FIG. 6D, schematically illustrating the Propionate metabolism (3-hydroxypropionate [30HProp]).

FIG. 6D schematically illustrates a biochemical network map of urine metabolites altered in diabetic kidney disease.

FIG. 6E illustrates in detailed area (magnified insert) B, of FIG. 6D, schematically illustrating pyrimidine metabolism (uridine).

FIG. 6F illustrates in detailed area (magnified insert) F, of FIG. 6D, schematically illustrating branched chain fatty acid metabolism (3-methyladipic acid [3MAdipic]).

FIG. 6G illustrates in detailed area (magnified insert) G, of FIG. 6D, schematically illustrating oxalate metabolism (glycolic acid).

FIG. 6H illustrates in detailed area (magnified insert) A, of FIG. 6D, schematically illustrating part of the Krebs cycle (citrate, aconitate).

FIG. 7 illustrates schematics illustrating a network map combining metabolites with enzymes that regulate its production. Proteins which interact with these enzymes are also shown (i.e. first neighbors of the enzymes on protein-protein interaction network). The map was drawn using Cytoscape.

FIG. 8A, FIG. 8B, FIG. 8C and FIG. 8D illustrate results showing that mitochondrial biogenesis is reduced in diabetic nephropathy:

FIG. 8A illustrates four images of immunostaining of cytochrome C oxidase, a representative immunostaining of cytochrome C oxidase (complex IV) subunit II staining in normal and diabetic kidney (40×mag).

FIG. 8B graphically illustrates data if a semi-quantitative analysis (n=5 per group, p<0.05).

FIG. 8C graphically illustrates data depicting the copy number of exosome-protected, DNase I-resistant mitochondrial DNA in urinary exosomes from patients with diabetic kidney disease (Diabetes) vs. healthy controls (Controls). (Controls 432+/−147 copies/ng; Diabetes 36+/−18 copies/ng: p<0.01, N=16 per group).

FIG. 8D graphically illustrates data demonstrating gene expression of PGClalpha from biopsy samples from pre-transplant biopsies (Control, n=8), patients with diabetic nephropathy (Diab Neph, n=14), minimal change disease (Min Change, n=6).

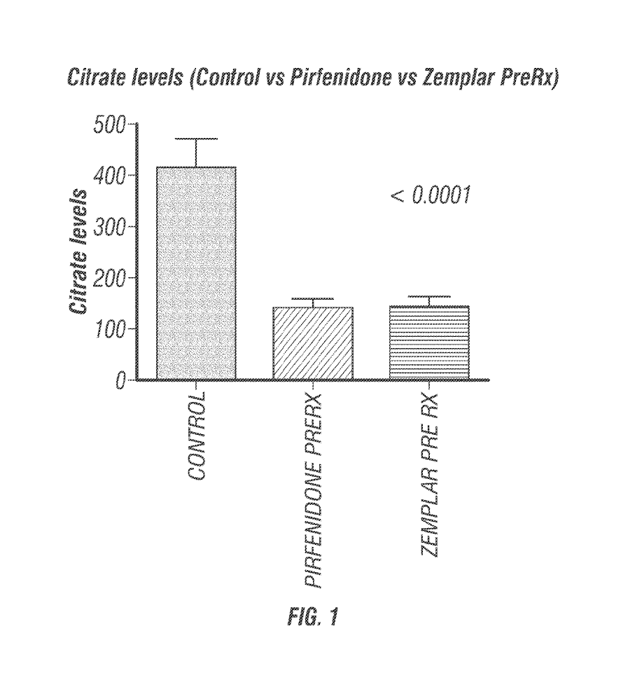

### DETAILED DESCRIPTION

The present technology provides relates to the identification of the presence of kidney disease, determination of the level of kidney disease, or the progression of kidney disease, in a subject that has or has not been diagnosed with diabetes. The present technology also provides methods for determining the efficacy of a treatment for kidney disease, and methods for determining the toxicity of a therapeutic to a subject's kidneys. The present technology further relates to the identification of biomarkers that indicate kidney disease, for example, diabetic kidney disease. The biomarkers reflect mitochondrial function and the overall health of the organ. The technology may, for example, also be used to identify a therapeutic for treating or preventing a kidney condition in the subject. It is appreciated that organic acids or 5-oxoproline readily obtained from blood, plasma, saliva, CSF, joint fluid, urine, as well as solid tissue biopsy. While urine is a sampling fluid in many embodiments of the technology owing to direct contact with the kidney, it is appreciated that other biological fluids have advantages in being sampled for other purposes and therefore allow for inventive determination of nephrological condition as part of a battery of tests performed on a single sample such as blood, plasma, serum, saliva, CSF, joint fluid or urine. It may be appreciated that the organic fluids may be detected using standard techniques, such as, for example, those presented herein, allowing for the varying methods for sample collection and initial processing.

A subject illustratively includes a dog, a cat, a horse, a cow, a pig, a sheep, a goat, a chicken, non-human primate, a human, a rat, and a mouse. Subjects may, for example, include those suspected of having or at risk for developing diabetic kidney disease, diabetes, chronic kidney disease.

“Marker” as used herein, refers to a small organic molecule or metabolite thereof which is differentially present in a sample taken from patients having kidney disease and/or or a proclivity for the disease as compared to a comparable sample taken from control subjects (e.g., a person with a negative diagnosis, normal or healthy subject) or from a historical value of the marker for the patient.

The phrase “kidney disease” as used herein indicates any disease or condition that affects the kidneys such as, for example, chronic kidney disease, acute kidney disease, congenital kidney disease, polycystic kidney disease, hypertensive kidney disease, inflammatory kidney disease, glomerulonephritis, tubulo-interstitial disease, and the like. Chronic kidney disease often manifests in such a way that there are no detectable symptoms until there is irreversible damage to the kidneys.

The terms “patient”, “individual” or “subject” are used interchangeably herein, and is meant a mammalian subject to be treated, for example, a human. In some cases, the processes of the present technology find use in experimental animals, in veterinary application, and in the development of vertebrate models for disease, including, but not limited to, rodents including mice, rats, and hamsters; birds, fish reptiles, and primates.

The terms “normal subject” and “healthy subject” refer to a mammalian subject, for example, a human, that is not or has not suffered from kidney disease and does not have a history of past kidney disease.

“Biological Sample” is used herein includes polynucleotides, polypeptides, peptides, antibodies fragments and correlateable breakdown products and is a bodily fluid; a soluble fraction of a cell preparation, or media in which cells are grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint; skin; or hair; and fragments of the aforementioned.

The term “diluent,” as used herein, refers to, for example, any composition added to the biological sample so that the sample may be analyzed according to the methods of the current technology such as, for example gas chromatography or mass spectrometry. Appropriate diluents are discussed herein, and for example, in Hartmann, S., et al., Clin Chem. 2006 June; 52(6):1127-37. Epub 2006 Apr. 13; and Barshop, B A, et al., Mol Genet Metab. 2000 January; 69(1):64-8.

The term “substrate,” as used herein, refers to, for example, any material or composition to which the biological sample may be bound, such as, for example, beads, solid surfaces, microtiter plates, wafers, antibodies, filters, concentrators, and the like.

The panel of biomarkers discussed herein may be able to indicate the overall health of the kidney. It appears to be independent of the blood creatinine and the urine protein. The measurements of these metabolites may, for example, be used to identify patients with a reduction in kidney function, possibly at an earlier stage than the blood creatinine marker. In another example, the panel of metabolites may be used to determine whether a new or existing drug is harmful or beneficial for the kidney.

The level of one or more organic acids or metabolites may be compared to a reference level of the particular organic acids or metabolites. The reference level may be determined from a biological sample obtained from the same subject at an earlier time period, for example, about 1, 2, 3, 4, 5, 6, 7 days or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 weeks, or 1, 2, 3, 4, 5, 6, 7, 8, 9, 10, 15, 20, or 25 years or more before the test biological sample is obtained. In other embodiments, two or more biological samples are obtained from a subject, and the levels of the organic acid or metabolite are determined in order to, for example, determining the progression of kidney disease, or another disease or condition discussed herein. The two or more biological samples may be obtained independently, from, for example, about 1, 2, 3, 4, 5, 6, 7 days or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 weeks, or 1, 2, 3, 4, 5, 6, 7, 8, 9, 10, 15, 20, or 25 years or more apart.

Alternatively, the reference level may be determined from a biological sample obtained from a healthy patient, before, at the same time, or after the biological sample is obtained from the subject. The reference level may be determined as an average of the levels of biological samples obtained from more than one healthy patient. The reference level may be determined by the same entity that determines the level of the organic acid or metabolite in the biological sample obtained from the subject. Or, the reference level may be a level known, or published, by another entity.

Optimized and Personalized Therapeutic Treatment

Treatment for kidney disease, for example, diabetic kidney disease, may be optimized by determining the concentration of glycolic acid, 3-OH isobutyric acid, 3-OH isovaleric acid, aconitic acid, homovanillic acid, citric acid, uracil, or glutaric acid, 5-oxoproline or a panel of these biomarkers, during the course of treatment. Different patients having different stages or types of kidney disease or diabetes, may react differently to various therapies. The response to treatment may be monitored by following the glycolic acid, 3-OH isobutyric acid, 3-OH isovaleric acid, aconitic acid, homovanillic acid, citric acid, uracil, 5-oxoproline or glutaric acid concentrations or levels in various body fluids or tissues. The determination of the concentration, level, or amount of a metabolite, such as, glycolic acid, 3-OH isobutyric acid, 3-OH isovaleric acid, aconitic acid, homovanillic acid, citric acid, uracil, or glutaric acid, may include detection by gas chromatography, mass spectrometry, or both in tandem, or other methods. Optimizing treatment for individual patients may help to avoid side effects as a result of overdosing, may help to determine when the treatment is ineffective and to change the course of treatment, or may help to determine when doses may be increased. Technology discussed herein optimizes therapeutic methods for treating kidney disease, for example, diabetic kidney disease, by allowing a clinician to track a biomarker, such as, for example, glycolic acid, 3-OH isobutyric acid, 3-OH isovaleric acid, aconitic acid, homovanillic acid, citric acid, uracil, 5-oxoproline or glutaric acid, or a panel of these biomarkers, and determine whether a subsequent dose of a drug or vaccine for administration to a subject may be maintained, reduced or increased, and to determine the timing for the subsequent dose. By a “panel” of biomarkers is meant at least two, three, four, five, six, seven, eight, nine, ten or eleven of the biomarkers selected from the group consisting of glycolic acid, 3-OH isobutyric acid, 3-OH isovaleric acid, aconitic acid, homovanillic acid, citric acid, uracil, 5-oxoproline and glutaric acid.

For example, the amount or concentration of certain biomarkers may change during the course of treatment of kidney disease. Predetermined target levels of such biomarkers, or biomarker thresholds may be identified in normal subject, which allow a clinician to determine whether a subsequent dose of a drug administered to a subject in need thereof, such as a subject with a kidney disease, such as, for example, diabetic kidney disease. Based on this determination, the treatment may be increased, decreased or maintained. A clinician can make such a determination based on whether the presence, absence or amount of a biomarker is below, above or about the same as a biomarker threshold, respectively, in certain embodiments.

For example, determining that an over-represented biomarker level is significantly reduced and/or that an under-represented biomarker level is significantly increased after drug treatment or vaccination provides an indication to a clinician that an administered drug is exerting a therapeutic effect. By “level” is meant the concentration of the biomarker in a fluid or tissue, or the absolute amount in a tissue. Based on such a biomarker determination, a clinician could make a decision to maintain a subsequent dose of the drug or raise or lower the subsequent dose, including modifying the timing of administration. The term “drug” or “therapeutic” includes traditional pharmaceuticals, such as small molecules, as well as biologics, such as nucleic acids, antibodies, proteins, polypeptides, modified cells and the like. In another example, determining that an over-represented biomarker level is not significantly reduced and/or that an under-represented biomarker level is not significantly increased provides an indication to a clinician that an administered drug is not significantly exerting a therapeutic effect. Based on such a biomarker determination, a clinician could make a decision to increase a subsequent dose of the drug. Given that drugs can be toxic to a subject and exert side effects, methods provided herein optimize therapeutic approaches as they provide the clinician with the ability to “dial in” an efficacious dosage of a drug and minimize side effects. In specific examples, methods provided herein allow a clinician to “dial up” the dose of a drug to a therapeutically efficacious level, where the dialed up dosage is below a toxic threshold level. Accordingly, treatment methods discussed herein enhance efficacy and reduce the likelihood of toxic side effects. Also, the methods discussed herein may be used to analyze the toxicity of a therapeutic not designed to treat kidney disease, but designed to treat another disease or condition. Using these methods, toxicity of the therapeutic to the kidney may be determined.

Sources of Biomarkers

The presence, absence or amount of a biomarker can be determined within a subject (e.g., in situ) or outside a subject (e.g., ex vivo). In some embodiments, presence, absence or amount of a biomarker can be determined in cells (e.g., differentiated cells, stem cells), and in certain embodiments, presence, absence or amount of a biomarker can be determined in a substantially cell-free medium (e.g., in vitro). The term “identifying the presence, absence or amount of a biomarker in a subject” as used herein refers to any method known in the art for assessing the biomarker and inferring the presence, absence or amount in the subject (e.g., in situ, ex vivo or in vitro methods).

A fluid or tissue sample often is obtained from a subject for determining presence, absence or amount of biomarker ex vivo. Non-limiting parts of the body from which a tissue sample may be obtained include leg, arm, abdomen, upper back, lower back, chest, hand, finger, fingernail, foot, toe, toenail, neck, rectum, nose, throat, mouth, scalp, face, spine, throat, heart, lung, breast, kidney, liver, intestine, colon, pancreas, bladder, cervix, testes, muscle, skin, hair, tumor or area surrounding a tumor, and the like, in some embodiments. A tissue sample can be obtained by any suitable method known in the art, including, without limitation, biopsy (e.g., shave, punch, incisional, excisional, curettage, fine needle aspirate, scoop, scallop, core needle, vacuum assisted, open surgical biopsies) and the like, in certain embodiments. Examples of a fluid that can be obtained from a subject includes, without limitation, blood, cerebrospinal fluid, spinal fluid, lavage fluid (e.g., bronchoalveolar, gastric, peritoneal, ductal, ear, arthroscopic), urine, interstitial fluid, feces, sputum, saliva, nasal mucous, prostate fluid, lavage, semen, lymphatic fluid, bile, tears, sweat, breast milk, breast fluid, fluid from region of inflammation, fluid from region of muscle wasting and the like, in some embodiments.

A sample from a subject may be processed prior to determining presence, absence or amount of a biomarker. For example, a blood sample from a subject may be processed to yield a certain fraction, including without limitation, plasma, serum, buffy coat, red blood cell layer and the like, and biomarker presence, absence or amount can be determined in the fraction. In certain embodiments, a tissue sample (e.g., tumor biopsy sample) can be processed by slicing the tissue sample and observing the sample under a microscope before and/or after the sliced sample is contacted with an agent that visualizes a biomarker (e.g., antibody). In some embodiments, a tissue sample can be exposed to one or more of the following non-limiting conditions: washing, exposure to high salt or low salt solution (e.g., hypertonic, hypotonic, isotonic solution), exposure to shearing conditions (e.g., sonication, press (e.g., French press)), mincing, centrifugation, separation of cells, separation of tissue and the like. In certain embodiments, a biomarker can be separated from tissue and the presence, absence or amount determined in vitro. A sample also may be stored for a period of time prior to determining the presence, absence or amount of a biomarker (e.g., a sample may be frozen, cryopreserved, maintained in a preservation medium (e.g., formaldehyde)).

A sample can be obtained from a subject at any suitable time of collection after a drug is delivered to the subject. For example, a sample may be collected within about one hour after a drug is delivered to a subject (e.g., within about 5, 10, 15, 20, 25, 30, 35, 40, 45, 55 or 60 minutes of delivering a drug), within about one day after a drug is delivered to a subject (e.g., within about 2, 3, 4, 5, 6, 7, 8, 9, 10, 11, 12, 13, 14, 15, 16, 17, 18, 19, 20, 21, 22, 23 or 24 hours of delivering a drug) or within about two weeks after a drug is delivered to a subject (e.g., within about 2, 3, 4, 5, 6, 7, 8, 9, 10, 11, 12, 13 or 14 days of delivering the drug). A collection may be made on a specified schedule including hourly, daily, semi-weekly, weekly, bi-weekly, monthly, bi-monthly, quarterly, and yearly, and the like, for example. If a drug is administered continuously over a time period (e.g., infusion), the delay may be determined from the first moment of drug is introduced to the subject, from the time the drug administration ceases, or a point in-between (e.g., administration time frame midpoint or other point).

Biomarker Detection

The presence, absence or amount of one or more biomarkers may be determined by any suitable method known in the art, and non-limiting determination methods are discussed herein. Determining the presence, absence or amount of a biomarker sometimes comprises use of a biological assay. In a biological assay, one or more signals detected in the assay can be converted to the presence, absence or amount of a biomarker. Converting a signal detected in the assay can comprise, for example, use of a standard curve, one or more standards (e.g., internal, external), a chart, a computer program that converts a signal to a presence, absence or amount of biomarker, and the like, and combinations of the foregoing.

Indication for Adjusting or Maintaining Subsequent Drug Dose

An indication for adjusting or maintaining a subsequent drug dose can be based on the presence or absence of a biomarker. For example, when (i) low sensitivity determinations of biomarker levels are available, (ii) biomarker levels shift sharply in response to a drug, (iii) low levels or high levels of biomarker are present, and/or (iv) a drug is not appreciably toxic at levels of administration, presence or absence of a biomarker can be sufficient for generating an indication of adjusting or maintaining a subsequent drug dose.

An indication for adjusting or maintaining a subsequent drug dose often is based on the amount or level of a biomarker. An amount of a biomarker can be a mean, median, nominal, range, interval, maximum, minimum, or relative amount, in some embodiments. An amount of a biomarker can be expressed with or without a measurement error window in certain embodiments. An amount of a biomarker in some embodiments can be expressed as a biomarker concentration, biomarker weight per unit weight, biomarker weight per unit volume, biomarker moles, biomarker moles per unit volume, biomarker moles per unit weight, biomarker weight per unit cells, biomarker volume per unit cells, biomarker moles per unit cells and the like. Weight can be expressed as femtograms, picograms, nanograms, micrograms, milligrams and grams, for example. Volume can be expressed as femtoliters, picoliters, nanoliters, microliters, milliliters and liters, for example. Moles can be expressed in picomoles, nanomoles, micromoles, millimoles and moles, for example. In some embodiments, unit weight can be weight of subject or weight of sample from subject, unit volume can be volume of sample from the subject (e.g., blood sample volume) and unit cells can be per one cell or per a certain number of cells (e.g., micrograms of biomarker per 1000 cells). In some embodiments, an amount of biomarker determined from one tissue or fluid can be correlated to an amount of biomarker in another fluid or tissue, as known in the art.

An indication for adjusting or maintaining a subsequent drug dose often is generated by comparing a determined level of biomarker in a subject to a predetermined level of biomarker. A predetermined level of biomarker sometimes is linked to a therapeutic or efficacious amount of drug in a subject, sometimes is linked to a toxic level of a drug, sometimes is linked to presence of a condition, sometimes is linked to a treatment midpoint and sometimes is linked to a treatment endpoint, in certain embodiments. A predetermined level of a biomarker sometimes includes time as an element, and in some embodiments, a threshold is a time-dependent signature.

For example, an organic acid level of about 0.2-fold less than a normal level, or less (e.g., about 0.3, 0.4, 0.5, 0.6, 0.7, 0.8, 0.9, 1.0, 1.25, 1.5, 1.75, 2, 2.5, 3, 3.5, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75-fold less than a normal level) may indicate that the dosage of the drug or the frequency of administration may be increased in a subsequent administration.

For example, an organic acid level of about 5% fold less than a normal level, or less (e.g., about 6%, 7%, &%, 9%, 10%, 15%, 20%, 25%, 30%, 35%, 40%, 45%, 50%, 55%, 60%, 65%, 70%, 75%, or 80% less than a normal level) may indicate that the dosage of the drug or the frequency of administration may be increased in a subsequent administration.

The term “dosage” is meant to include both the amount of the dose and the frequency of administration, such as, for example, the timing of the next dose.

Some treatment methods comprise (i) administering a drug to a subject in one or more administrations (e.g., 1, 2, 3, 4, 5, 6, 7, 8, 9 or 10 doses), (ii) determining the presence, absence or amount of a biomarker in or from the subject after (i), (iii) providing an indication of increasing, decreasing or maintaining a subsequent dose of the drug for administration to the subject, and (iv) optionally administering the subsequent dose to the subject, where the subsequent dose is increased, decreased or maintained relative to the earlier dose(s) in (i). In some embodiments, presence, absence or amount of a biomarker is determined after each dose of drug has been administered to the subject, and sometimes presence, absence or amount of a biomarker is not determined after each dose of the drug has been administered (e.g., a biomarker is assessed after one or more of the first, second, third, fourth, fifth, sixth, seventh, eighth, ninth or tenth dose, but not assessed every time after each dose is administered).

An indication for adjusting a subsequent drug dose can be considered a need to increase or a need to decrease a subsequent drug dose. An indication for adjusting or maintaining a subsequent drug dose can be considered by a clinician, and the clinician may act on the indication in certain embodiments. In some embodiments, a clinician may opt not to act on an indication. Thus, a clinician can opt to adjust or not adjust a subsequent drug dose based on the indication provided.

An indication of adjusting or maintaining a subsequent drug dose, and/or the subsequent drug dosage, can be provided in any convenient manner. An indication may be provided in tabular form (e.g., in a physical or electronic medium) in some embodiments. For example, a biomarker threshold may be provided in a table, and a clinician may compare the presence, absence or amount of the biomarker determined for a subject to the threshold. The clinician then can identify from the table an indication for subsequent drug dose. In certain embodiments, an indication can be presented (e.g., displayed) by a computer after the presence, absence or amount of a biomarker is provided to computer (e.g., entered into memory on the computer). For example, presence, absence or amount of a biomarker determined for a subject can be provided to a computer (e.g., entered into computer memory by a user or transmitted to a computer via a remote device in a computer network), and software in the computer can generate an indication for adjusting or maintaining a subsequent drug dose, and/or provide the subsequent drug dose amount. A subsequent dose can be determined based on certain factors other than biomarker presence, absence or amount, such as weight of the subject, one or more metabolite levels for the subject (e.g., metabolite levels pertaining to liver function) and the like, for example.

Once a subsequent dose is determined based on the indication, a clinician may administer the subsequent dose or provide instructions to adjust the dose to another person or entity. The term “clinician” as used herein refers to a decision maker, and a clinician is a medical professional in certain embodiments. A decision maker can be a computer or a displayed computer program output in some embodiments, and a health service provider may act on the indication or subsequent drug dose displayed by the computer. A decision maker may administer the subsequent dose directly (e.g., infuse the subsequent dose into the subject) or remotely (e.g., pump parameters may be changed remotely by a decision maker).

A subject can be prescreened to determine whether or not the presence, absence or amount of a particular biomarker may be determined. Non-limiting examples of prescreens include identifying the presence or absence of a genetic marker (e.g., polymorphism, particular nucleotide sequence); identifying the presence, absence or amount of a particular metabolite. A prescreen result can be used by a clinician in combination with the presence, absence or amount of a biomarker to determine whether a subsequent drug dose may be adjusted or maintained.

### Identification of the Presence of Kidney Disease

The present technology provides relates to the identification of the presence of kidney disease, determination of the level of kidney disease, or the progression of kidney disease, in a subject that has or has not been diagnosed with diabetes. The present technology also provides methods for determining the efficacy of a treatment for kidney disease, and methods for determining the toxicity of a therapeutic to a subject's kidneys. The present technology further relates to the identification of biomarkers that indicate kidney disease, for example, diabetic kidney disease. The biomarkers reflect mitochondrial function and the overall health of the organ. The technology may, for example, also be used to identify a therapeutic for treating or preventing a kidney condition in the subject. It is appreciated that metabolites such as organic acids may be readily obtained from blood, plasma, saliva, CSF, joint fluid, urine, as well as solid tissue biopsy. While urine is a sampling fluid in many embodiments of the technology owing to direct contact with the kidney, it is appreciated that other biological fluids have advantages in being sampled for other purposes and therefore allow for inventive determination of nephrological condition as part of a battery of tests performed on a single sample such as blood, plasma, serum, saliva, CSF, joint fluid or urine. It may be appreciated that the organic fluids may be detected using standard techniques, such as, for example, those presented herein, allowing for the varying methods for sample collection and initial processing.

A subject illustratively includes a dog, a cat, a horse, a cow, a pig, a sheep, a goat, a chicken, non-human primate, a human, a rat, and a mouse. Subjects may, for example, include those suspected of having or at risk for developing diabetic kidney disease, diabetes, chronic kidney disease.

“Marker” as used herein, refers to a small organic molecule or metabolite thereof which is differentially present in a sample taken from patients having kidney disease and/or or a proclivity for the disease as compared to a comparable sample taken from control subjects (e.g., a person with a negative diagnosis, normal or healthy subject) or from a historical value of the marker for the patient.

The phrase “kidney disease” as used herein indicates any disease or condition that affects the kidneys such as, for example, chronic kidney disease, acute kidney disease, congenital kidney disease, polycystic kidney disease, hypertensive kidney disease, inflammatory kidney disease, glomerulonephritis, tubulo-interstitial disease, and the like. Chronic kidney disease often manifests in such a way that there are no detectable symptoms until there is irreversible damage to the kidneys.

The terms “patient”, “individual” or “subject” are used interchangeably herein, and is meant a mammalian subject to be treated, for example, a human. In some cases, the processes of the present technology find use in experimental animals, in veterinary application, and in the development of vertebrate models for disease, including, but not limited to, rodents including mice, rats, and hamsters; birds, fish reptiles, and primates.

The terms “normal subject” and “healthy subject” refer to a mammalian subject, for example, a human, that is not or has not suffered from kidney disease and does not have a history of past kidney disease.

“Biological Sample” is used herein includes polynucleotides, polypeptides, peptides, antibodies fragments and correlateable breakdown products and is a bodily fluid; a soluble fraction of a cell preparation, or media in which cells are grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint; skin; or hair; and fragments of the aforementioned.

“Additional” as in “additional metabolite” or “additional organic acid” is meant a metabolite or organic acid other than the “at least one” or one metabolite or organic acid selected from the group consisting of 3-methyl adipic acid, 2-methyl acetoacetate, 3-methyl crotonyl glycine, 3-hydroxy propionate, 2-ethyl 3-OH propionate, and tiglylglycine. The “additional” metabolite or organic acid may, or may not be selected from the group consisting of 3-methyl adipic acid, 2-methyl acetoacetate, 3-methyl crotonyl glycine, 3-hydroxy propionate, 2-ethyl 3-OH propionate, and tiglylglycine, however it is not meant to be the same metabolite or organic acid already selected.

The term “diluent,” as used herein, refers to, for example, any composition added to the biological sample so that the sample may be analyzed according to the methods of the current technology such as, for example gas chromatography or mass spectrometry. Appropriate diluents are discussed herein, and for example, in Hartmann, S., et al., Clin Chem. 2006 June; 52(6):1127-37. Epub 2006 Apr. 13; and Barshop, B A, et al., Mol Genet Metab. 2000 January; 69(1):64-8.

The term “substrate,” as used herein, refers to, for example, any material or composition to which the biological sample may be bound, such as, for example, beads, solid surfaces, microtiter plates, wafers, antibodies, filters, concentrators, and the like.

The panel of biomarkers discussed herein may be able to indicate the overall health of the kidney. It appears to be independent of the blood creatinine and the urine protein. The measurements of these metabolites may, for example, be used to identify patients with a reduction in kidney function, possibly at an earlier stage than the blood creatinine marker. In another example, the panel of metabolites may be used to determine whether a new or existing drug is harmful or beneficial for the kidney.

The level of one or more organic acids or metabolites may be compared to a reference level of the particular organic acids or metabolites. The reference level may be determined from a biological sample obtained from the same subject at an earlier time period, for example, about 1, 2, 3, 4, 5, 6, 7 days or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 weeks, or 1, 2, 3, 4, 5, 6, 7, 8, 9, 10, 15, 20, or 25 years or more before the test biological sample is obtained. In other embodiments, two or more biological samples are obtained from a subject, and the levels of the organic acid or metabolite are determined in order to, for example, determining the progression of kidney disease, or another disease or condition discussed herein. The two or more biological samples may be obtained independently, from, for example, about 1, 2, 3, 4, 5, 6, 7 days or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 weeks, or 1, 2, 3, 4, 5, 6, 7, 8, 9, 10, 15, 20, or 25 years or more apart.

Alternatively, the reference level may be determined from a biological sample obtained from a healthy patient, before, at the same time, or after the biological sample is obtained from the subject. The reference level may be determined as an average of the levels of biological samples obtained from more than one healthy patient. The reference level may be determined by the same entity that determines the level of the organic acid or metabolite in the biological sample obtained from the subject. Or, the reference level may be a level known, or published, by another entity.

Optimized and Personalized Therapeutic Treatment

Treatment for kidney disease, for example, diabetic kidney disease, may be optimized by determining the concentration of glycolic acid, 3-OH isobutyric acid, 3-OH isovaleric acid, aconitic acid, homovanillic acid, citric acid, uracil, or glutaric acid, 5-oxoproline or a panel of these biomarkers, during the course of treatment. Different patients having different stages or types of kidney disease or diabetes, may react differently to various therapies. The response to treatment may be monitored by following the glycolic acid, 3-OH isobutyric acid, 3-OH isovaleric acid, aconitic acid, homovanillic acid, citric acid, uracil, 5-oxoproline or glutaric acid concentrations or levels in various body fluids or tissues. The determination of the concentration, level, or amount of a metabolite, such as, glycolic acid, 3-OH isobutyric acid, 3-OH isovaleric acid, aconitic acid, homovanillic acid, citric acid, uracil, or glutaric acid, may include detection by gas chromatography, mass spectrometry, or both in tandem, or other methods. Optimizing treatment for individual patients may help to avoid side effects as a result of overdosing, may help to determine when the treatment is ineffective and to change the course of treatment, or may help to determine when doses may be increased. Technology discussed herein optimizes therapeutic methods for treating kidney disease, for example, diabetic kidney disease, by allowing a clinician to track a biomarker, such as, for example, glycolic acid, 3-OH isobutyric acid, 3-OH isovaleric acid, aconitic acid, homovanillic acid, citric acid, uracil, 5-oxoproline or glutaric acid, or a panel of these biomarkers, and determine whether a subsequent dose of a drug or vaccine for administration to a subject may be maintained, reduced or increased, and to determine the timing for the subsequent dose. By a “panel” of biomarkers is meant at least two, three, four, five, six, seven, eight, nine, ten, eleven, twelve, or thirteen of the biomarkers selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine.

For example, the amount or concentration of certain biomarkers may change during the course of treatment of kidney disease. Predetermined target levels of such biomarkers, or biomarker thresholds may be identified in normal subject, which allow a clinician to determine whether a subsequent dose of a drug administered to a subject in need thereof, such as a subject with a kidney disease, such as, for example, diabetic kidney disease. Based on this determination, the treatment may be increased, decreased or maintained. A clinician can make such a determination based on whether the presence, absence or amount of a biomarker is below, above or about the same as a biomarker threshold, respectively, in certain embodiments.

For example, determining that an over-represented biomarker level is significantly reduced and/or that an under-represented biomarker level is significantly increased after drug treatment or vaccination provides an indication to a clinician that an administered drug is exerting a therapeutic effect. By “level” is meant the concentration of the biomarker in a fluid or tissue, or the absolute amount in a tissue. Based on such a biomarker determination, a clinician could make a decision to maintain a subsequent dose of the drug or raise or lower the subsequent dose, including modifying the timing of administration. The term “drug” or “therapeutic” includes traditional pharmaceuticals, such as small molecules, as well as biologics, such as nucleic acids, antibodies, proteins, polypeptides, modified cells and the like. In another example, determining that an over-represented biomarker level is not significantly reduced and/or that an under-represented biomarker level is not significantly increased provides an indication to a clinician that an administered drug is not significantly exerting a therapeutic effect. Based on such a biomarker determination, a clinician could make a decision to increase a subsequent dose of the drug. Given that drugs can be toxic to a subject and exert side effects, methods provided herein optimize therapeutic approaches as they provide the clinician with the ability to “dial in” an efficacious dosage of a drug and minimize side effects. In specific examples, methods provided herein allow a clinician to “dial up” the dose of a drug to a therapeutically efficacious level, where the dialed up dosage is below a toxic threshold level. Accordingly, treatment methods discussed herein enhance efficacy and reduce the likelihood of toxic side effects. Also, the methods discussed herein may be used to analyze the toxicity of a therapeutic not designed to treat kidney disease, but designed to treat another disease or condition. Using these methods, toxicity of the therapeutic to the kidney may be determined.

Sources of Biomarkers

The presence, absence or amount of a biomarker can be determined within a subject (e.g., in situ) or outside a subject (e.g., ex vivo). In some embodiments, presence, absence or amount of a biomarker can be determined in cells (e.g., differentiated cells, stem cells), and in certain embodiments, presence, absence or amount of a biomarker can be determined in a substantially cell-free medium (e.g., in vitro). The term “identifying the presence, absence or amount of a biomarker in a subject” as used herein refers to any method known in the art for assessing the biomarker and inferring the presence, absence or amount in the subject (e.g., in situ, ex vivo or in vitro methods).

A fluid or tissue sample often is obtained from a subject for determining presence, absence or amount of biomarker ex vivo. Non-limiting parts of the body from which a tissue sample may be obtained include leg, arm, abdomen, upper back, lower back, chest, hand, finger, fingernail, foot, toe, toenail, neck, rectum, nose, throat, mouth, scalp, face, spine, throat, heart, lung, breast, kidney, liver, intestine, colon, pancreas, bladder, cervix, testes, muscle, skin, hair, tumor or area surrounding a tumor, and the like, in some embodiments. A tissue sample can be obtained by any suitable method known in the art, including, without limitation, biopsy (e.g., shave, punch, incisional, excisional, curettage, fine needle aspirate, scoop, scallop, core needle, vacuum assisted, open surgical biopsies) and the like, in certain embodiments. Examples of a fluid that can be obtained from a subject includes, without limitation, blood, cerebrospinal fluid, spinal fluid, lavage fluid (e.g., bronchoalveolar, gastric, peritoneal, ductal, ear, arthroscopic), urine, interstitial fluid, feces, sputum, saliva, nasal mucous, prostate fluid, lavage, semen, lymphatic fluid, bile, tears, sweat, breast milk, breast fluid, fluid from region of inflammation, fluid from region of muscle wasting and the like, in some embodiments.

A sample from a subject may be processed prior to determining presence, absence or amount of a biomarker. For example, a blood sample from a subject may be processed to yield a certain fraction, including without limitation, plasma, serum, buffy coat, red blood cell layer and the like, and biomarker presence, absence or amount can be determined in the fraction. In certain embodiments, a tissue sample (e.g., tumor biopsy sample) can be processed by slicing the tissue sample and observing the sample under a microscope before and/or after the sliced sample is contacted with an agent that visualizes a biomarker (e.g., antibody). In some embodiments, a tissue sample can be exposed to one or more of the following non-limiting conditions: washing, exposure to high salt or low salt solution (e.g., hypertonic, hypotonic, isotonic solution), exposure to shearing conditions (e.g., sonication, press (e.g., French press)), mincing, centrifugation, separation of cells, separation of tissue and the like. In certain embodiments, a biomarker can be separated from tissue and the presence, absence or amount determined in vitro. A sample also may be stored for a period of time prior to determining the presence, absence or amount of a biomarker (e.g., a sample may be frozen, cryopreserved, maintained in a preservation medium (e.g., formaldehyde)).

A sample can be obtained from a subject at any suitable time of collection after a drug is delivered to the subject. For example, a sample may be collected within about one hour after a drug is delivered to a subject (e.g., within about 5, 10, 15, 20, 25, 30, 35, 40, 45, 55 or 60 minutes of delivering a drug), within about one day after a drug is delivered to a subject (e.g., within about 2, 3, 4, 5, 6, 7, 8, 9, 10, 11, 12, 13, 14, 15, 16, 17, 18, 19, 20, 21, 22, 23 or 24 hours of delivering a drug) or within about two weeks after a drug is delivered to a subject (e.g., within about 2, 3, 4, 5, 6, 7, 8, 9, 10, 11, 12, 13 or 14 days of delivering the drug). A collection may be made on a specified schedule including hourly, daily, semi-weekly, weekly, bi-weekly, monthly, bi-monthly, quarterly, and yearly, and the like, for example. If a drug is administered continuously over a time period (e.g., infusion), the delay may be determined from the first moment of drug is introduced to the subject, from the time the drug administration ceases, or a point in-between (e.g., administration time frame midpoint or other point).

Biomarker Detection

The presence, absence or amount of one or more biomarkers may be determined by any suitable method known in the art, and non-limiting determination methods are discussed herein. Determining the presence, absence or amount of a biomarker sometimes comprises use of a biological assay. In a biological assay, one or more signals detected in the assay can be converted to the presence, absence or amount of a biomarker. Converting a signal detected in the assay can comprise, for example, use of a standard curve, one or more standards (e.g., internal, external), a chart, a computer program that converts a signal to a presence, absence or amount of biomarker, and the like, and combinations of the foregoing.

Indication for Adjusting or Maintaining Subsequent Drug Dose

An indication for adjusting or maintaining a subsequent drug dose can be based on the presence or absence of a biomarker. For example, when (i) low sensitivity determinations of biomarker levels are available, (ii) biomarker levels shift sharply in response to a drug, (iii) low levels or high levels of biomarker are present, and/or (iv) a drug is not appreciably toxic at levels of administration, presence or absence of a biomarker can be sufficient for generating an indication of adjusting or maintaining a subsequent drug dose.

An indication for adjusting or maintaining a subsequent drug dose often is based on the amount or level of a biomarker. An amount of a biomarker can be a mean, median, nominal, range, interval, maximum, minimum, or relative amount, in some embodiments. An amount of a biomarker can be expressed with or without a measurement error window in certain embodiments. An amount of a biomarker in some embodiments can be expressed as a biomarker concentration, biomarker weight per unit weight, biomarker weight per unit volume, biomarker moles, biomarker moles per unit volume, biomarker moles per unit weight, biomarker weight per unit cells, biomarker volume per unit cells, biomarker moles per unit cells and the like. Weight can be expressed as femtograms, picograms, nanograms, micrograms, milligrams and grams, for example. Volume can be expressed as femtoliters, picoliters, nanoliters, microliters, milliliters and liters, for example. Moles can be expressed in picomoles, nanomoles, micromoles, millimoles and moles, for example. In some embodiments, unit weight can be weight of subject or weight of sample from subject, unit volume can be volume of sample from the subject (e.g., blood sample volume) and unit cells can be per one cell or per a certain number of cells (e.g., micrograms of biomarker per 1000 cells). In some embodiments, an amount of biomarker determined from one tissue or fluid can be correlated to an amount of biomarker in another fluid or tissue, as known in the art.

An indication for adjusting or maintaining a subsequent drug dose often is generated by comparing a determined level of biomarker in a subject to a predetermined level of biomarker. A predetermined level of biomarker sometimes is linked to a therapeutic or efficacious amount of drug in a subject, sometimes is linked to a toxic level of a drug, sometimes is linked to presence of a condition, sometimes is linked to a treatment midpoint and sometimes is linked to a treatment endpoint, in certain embodiments. A predetermined level of a biomarker sometimes includes time as an element, and in some embodiments, a threshold is a time-dependent signature.

For example, an organic acid or metabolite level of about 0.2-fold less than a normal level, or less (e.g., about 0.3, 0.4, 0.5, 0.6, 0.7, 0.8, 0.9, 1.0, 1.25, 1.5, 1.75, 2, 2.5, 3, 3.5, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75-fold less than a normal level) may indicate that the dosage of the drug or the frequency of administration may be increased in a subsequent administration.

For example, an organic acid or metabolite level of about 5% fold less than a normal level, or less (e.g., about 6%, 7%, &%, 9%, 10%, 15%, 20%, 25%, 30%, 35%, 40%, 45%, 50%, 55%, 60%, 65%, 70%, 75%, or 80% less than a normal level) may indicate that the dosage of the drug or the frequency of administration may be increased in a subsequent administration.

The term “dosage” is meant to include both the amount of the dose and the frequency of administration, such as, for example, the timing of the next dose.

Some treatment methods comprise (i) administering a drug to a subject in one or more administrations (e.g., 1, 2, 3, 4, 5, 6, 7, 8, 9 or 10 doses), (ii) determining the presence, absence or amount of a biomarker in or from the subject after (i), (iii) providing an indication of increasing, decreasing or maintaining a subsequent dose of the drug for administration to the subject, and (iv) optionally administering the subsequent dose to the subject, where the subsequent dose is increased, decreased or maintained relative to the earlier dose(s) in (i). In some embodiments, presence, absence or amount of a biomarker is determined after each dose of drug has been administered to the subject, and sometimes presence, absence or amount of a biomarker is not determined after each dose of the drug has been administered (e.g., a biomarker is assessed after one or more of the first, second, third, fourth, fifth, sixth, seventh, eighth, ninth or tenth dose, but not assessed every time after each dose is administered).

An indication for adjusting a subsequent drug dose can be considered a need to increase or a need to decrease a subsequent drug dose. An indication for adjusting or maintaining a subsequent drug dose can be considered by a clinician, and the clinician may act on the indication in certain embodiments. In some embodiments, a clinician may opt not to act on an indication. Thus, a clinician can opt to adjust or not adjust a subsequent drug dose based on the indication provided.

An indication of adjusting or maintaining a subsequent drug dose, and/or the subsequent drug dosage, can be provided in any convenient manner. An indication may be provided in tabular form (e.g., in a physical or electronic medium) in some embodiments. For example, a biomarker threshold may be provided in a table, and a clinician may compare the presence, absence or amount of the biomarker determined for a subject to the threshold. The clinician then can identify from the table an indication for subsequent drug dose. In certain embodiments, an indication can be presented (e.g., displayed) by a computer after the presence, absence or amount of a biomarker is provided to computer (e.g., entered into memory on the computer). For example, presence, absence or amount of a biomarker determined for a subject can be provided to a computer (e.g., entered into computer memory by a user or transmitted to a computer via a remote device in a computer network), and software in the computer can generate an indication for adjusting or maintaining a subsequent drug dose, and/or provide the subsequent drug dose amount. A subsequent dose can be determined based on certain factors other than biomarker presence, absence or amount, such as weight of the subject, one or more metabolite levels for the subject (e.g., metabolite levels pertaining to liver function) and the like, for example.

Once a subsequent dose is determined based on the indication, a clinician may administer the subsequent dose or provide instructions to adjust the dose to another person or entity. The term “clinician” as used herein refers to a decision maker, and a clinician is a medical professional in certain embodiments. A decision maker can be a computer or a displayed computer program output in some embodiments, and a health service provider may act on the indication or subsequent drug dose displayed by the computer. A decision maker may administer the subsequent dose directly (e.g., infuse the subsequent dose into the subject) or remotely (e.g., pump parameters may be changed remotely by a decision maker).

A subject can be prescreened to determine whether or not the presence, absence or amount of a particular biomarker may be determined. Non-limiting examples of prescreens include identifying the presence or absence of a genetic marker (e.g., polymorphism, particular nucleotide sequence); identifying the presence, absence or amount of a particular metabolite. A prescreen result can be used by a clinician in combination with the presence, absence or amount of a biomarker to determine whether a subsequent drug dose may be adjusted or maintained.

The following examples, and the figures, are intended to clarify the invention, and to demonstrate and further illustrate certain preferred embodiments and aspects without restricting the subject of the invention to the examples and figures.

### EXAMPLES

The examples set forth below illustrate certain embodiments and do not limit the technology.

### Example 1

### Urine Metabolite Profiles of Diabetic Patients

The urine metabolite profile of diabetic patients with kidney disease was studied to help identify organic acid derangements that can serve as biomarkers. The study population included 14 patients with a diagnosis of diabetic kidney disease with Chronic Kidney disease (D-CKD) 3-4 (Mean GFR 31.79+/−7.329). This population was compared to a control of 23 healthy volunteers with no diabetes or kidney disease. The control group contained 6 (26%) and the diabetic kidney disease with Chronic Kidney disease group contained 5 (35%) females.

Twenty-four hour urine was collected from both control and D-CKD subjects. A composite quantitative urine organic acid estimation was done using the Agilent 5973 Glass Chromatography and Mass spectrometry. Seventy-six different organic acids were looked at per sample and the results were standardized per mmol of creatinine.

The two groups were compared for each of the seventy-six metabolites and found 11 significant metabolites using the unpaired t test. A cut-off p value of P<0.00846 was chosen to have a false detection rate less than 0.05 to account for multiple testing. The panel of metabolites was reported as the amount of metabolite per standard amount of creatinine. Glomerular filtration rate (GFR), albumin creatinine ratio (ACR) and protein creatinine ratio (PCR) of the Diabetes with Chronic Kidney Disease group were then correlated to the values of the eleven significant metabolites using linear regression analysis.

The organic acid profile of urine from Diabetes with Chronic Kidney Disease patients was significantly decreased in metabolites of intermediate pathway. The following organic acids were significantly decreased in the Diabetes with Chronic Kidney Disease group, when compared to the healthy controls: glycolic acid, 3-OH isobutyric acid, 3-OH isovaleric acid, aconitic acid, homovanillic acid, citric acid, uracil. The level of 5-oxoproline was significantly increased in the Diabetes with Chronic Kidney Disease group when compared to the controls. Of these metabolites, only the citric acid levels and 3-OH isovaleric acid correlated to albumin creatinine ratio but not to GFR.

Using a second cohort of patients that were enrolled in a separate trial, 6 of 9 metabolites demonstrated significance. One of these metabolites, citrate, showed an increase with an interventional treatment (pirfenidone). The finding that this metabolite increases with treatment suggests that this biomarker may predict response to the drug and allow for a better guided treatment plan.

### Example 2

### Clinical Trial of the Therapeutic Pirfenidone for Diabetic Nephropathy

Urine metabolomics may be used to identify biomarkers for predicting responses to a drug. The testing and analysis of various drugs may be performed using methods similar to those provided in this example of the drug pirfenidone. As an example how urine metabolomics could provide information on the function of a drug in a clinical trial, the following is presented.

Pirfenidone is an orally-available anti-fibrotic agent that has shown efficacy in animal models of diabetic nephropathy and human focal segmental glomerulosclerosis. A randomized double-blind, placebo-controlled exploratory trial of pirfenidone was performed in subjects with diabetic nephropathy with elevated albuminura and reduced glomerular filtration rate (eGFR) (20-75 ml/min per 1.73 m²). The pre-specified primary outcome was eGFR change after one year of therapy. 78 subjects were randomly allocated to placebo (n=27), pirfenidone 1200 mg/d (n=26), or pirfenidone 2400 mg/d (n=25). 52 subjects completed the study. During the course of the study, eGFR increased with pirfenidone 1200 mg/d [+3.3±8.5 ml/min per 1.73 m² (mean±SD)], whereas eGFR fell with placebo (−2.2±4.8 ml/min per 1.73 m², p<0.03 versus pirfenidone 1200 mg/d) and with pirfenidone 2400 mg/d (−1.9±6.7 ml/min per 1.73 m², P=0.25 versus placebo). Of the initial 77 subjects, 5 initiated hemodialysis during the study: 4 in the placebo group, 0 in the pirfenidone 1200 mg group and 1 in the pirfenidone 2400 mg group (P=0.25). Baseline serum albumin levels were statistically associated with eGFR improvement in the entire cohort (p=0.001) and in the pirfenidone 2400 group (+0.9 [95% CI 0.7 to 1.1], p=0.03). Baseline levels of serum biomarkers of inflammation and fibrosis (IFN-gamma, TNF, soluble TNFR1, FGF23, and YKL-40) were significantly and directly correlated with eGFR change at one year but did not predict response to therapy. These results suggest that pirfenidone is a promising agent for larger studies in individuals with overt diabetic nephropathy.

Diabetic nephropathy remains the leading cause of end-stage kidney disease (ESKD) in the US, accounting for over 40% of incident ESKD cases. Diabetic nephropathy is characterized by inflammation, accumulation of mesangial matrix in established disease, marked tubulointerstitial fibrosis and vascular hyalinosis in advanced disease. Both mesangial matrix expansion and tubulointerstitial fibrosis correlate with progression of diabetic nephropathy to ESKD 1-3. The standard of care for diabetic nephropathy has been the use of inhibitors of the renin-angiotensin system (RAS), including angiotensin converting enzyme inhibitors (ACE-i) 4 and angiotensin II receptor blockers (ARBs) 5 6, and tight glycemic control. Blood pressure-independent benefits of RAS inhibitors may contribute to renal protection, possibly via inhibiting pro-fibrotic factors, such as transforming growth factor-beta (TGF-β) 7. However, the intensive use of RAS inhibitors is often limited by severe hyperkalemia, further reduction in the systemic blood pressure, and decreased renal blood flow. Even when maximized, they may decrease rate of progression, but do not arrest or reverse diabetic nephropathy. In addition, a recent large randomized clinical study found that combined ACE-i/ARB therapy was associated with worse renal outcomes in both diabetic and non-diabetic individuals without severe nephropathy at baseline 8. Therefore, novel approaches that block progression of nephropathy and do not rely on blocking the RAS axis may provide important additional therapies to block progressive diabetic nephropathy and renal failure.

Several growth factors or cytokines that are locally produced in the kidney appear to contribute to the extracellular matrix accumulation, inflammation and scarring in progressive diabetic nephropathy. The TGF-β system is activated and plays a pathogenetic role in diabetic kidney disease in animal models of both type 1 9 and type 2 diabetes 10. In addition, several studies in patients with both type 1 and type 2 diabetes indicate increased renal production of TGF-β 11, 12. The TNF-alpha system has also been recently linked with human diabetic nephropathy based on circulating blood levels 13 and gene expression in kidneys from patients with diabetic nephropathy 14. An orally bioavailable compound, pirfenidone, has been found to inhibit TGF-θ production and consequent matrix deposition in experimental animal models of lung and kidney disease 15,16. In animal models and cell culture studies, pirfenidone also reduces TNF-alpha-production 17,18. Oral pirfenidone administered to db/db mice after the onset of established diabetic kidney disease was effective in reducing glomerulosclerosis 19. In addition, in an open label clinical study in patients with advanced and treatment-refractory focal segmental sclerosis there was a 25% reduction in the rate of estimated glomerular filtration rate (eGFR) decline in patients on pirfenidone as compared to the rate of decline before pirfenidone 20.

It was hypothesized that administration of pirfenidone to type 1 and type 2 diabetic patients with established diabetic nephropathy would slow the rate of eGFR decline. A double-blind, placebo-controlled exploratory trial was performed, using a dose ranging protocol, as the optimal dose of pirfenidone has not been established.

### Subjects

Seventy-eight (78) subjects with diabetes, reduced eGFR and proteinuria were randomized to one of the three study arms (Table 1). Fifty-two (52) subjects completed 54 weeks of the trial. There were no statistically significant differences in demographic variables or kidney function at baseline between the completer group (n=52) and the non-completer group (n=26) (Supplemental Table 1). Of the 52 study completers baseline variables were similar across the three treatment groups except for higher diastolic blood pressure in the prifenidone 2400 mg group and higher serum albumin in the pirfenidone 1200 mg group (Table 1). Group analysis presented involves only study completers, either as a study group or as treatment group, except when data is specifically mentioned as pertaining to all enrolled subjects.

### Primary Endpoint

There was a significant difference in eGFR change from baseline to end-of-study in the pirfenidone 1200 mg group compared to placebo (Table 2). The mean inter-group difference in eGFR change was +5.5 ml/min per 1.73 m² (−2.2 ml/min per 1.73 m² in placebo vs. +3.3 ml/min per 1.73 m2 pirfenidone 1200 mg), 95% confidence interval (CI) 1.1, 9.9 ml/min per 1.73 m², P=0.03) A post-hoc two sample t-test of changes from baseline to month 6 also detected a significant difference between pirfenidone 1200 mg and placebo groups (5.3 ml/min/1.73 m² [CI 1.3 to 9.3] ml/min per 1.73 m²; p=0.02). The mean difference in eGFR change between the pirfenidone 2400 mg and placebo groups was 0.3 ml/min per 1.73 m² (CI −3.7, 4.2 ml/min per 1.73 m2), P=0.89.

In view of the extent of missing data, which might put in jeopardy the assumption of independently distributed data, both permutation tests and ANCOVA were performed with re-weighted least squares (IWLS), with controlling for baseline values and their interaction with treatment. The significance of comparison of eGFR change for pirfenidone 1200 mg versus placebo comparison was confirmed by the permutation test (P=0.012) and the ANCOVA with IWLS (P=0.019). Baseline characteristics of completers versus non-completers were found to be balanced for clinical variables shown in Supplemental Table 1 with no differences reaching statistical significance. General estimating equation (GEE) analysis of eGFR values as predictors of dropout at the next visit also did not refute the assumption that the missing data was missing at random.

Five subjects required dialysis during the course of the study; four subjects were in the placebo group and one subject was in the pirfenidone 2400 mg group. These subjects were considered to have completed the study at the time of dialysis initiation, and subjects who required dialysis were subsequently removed from the study. Imputing the eGFR as 10 ml/min per 1.73 m² for subjects requiring dialysis revealed an even greater effect of the pirfenidone 1200 mg/d regimen to slow the eGFR decline (Table 2).

### Secondary Outcomes

Urine albumin/creatinine ratio (ACR) was evaluated as a secondary outcome. There were no significant differences among study groups in change in ACR from baseline to the end of study
(P value across treatment groups=0.19). No significant change in urine TGF-beta levels was found within the placebo or treatment groups over 54 weeks. Urine TGF-beta levels increased by 1.4 pg/mg creatinine (95% CI 0.2, 6.6) over 12 months in the placebo group, numerically increased by 0.3 pg/mg (95% CI −1.6, 5.6) in the pirfenidone 1200 mg group, and numerically declined by 0.1 pg/mg (95% CI −3.4, 3.7) in the pirfenidone 2400 mg group (all P values >0.05).

### Predictors of Response to Pirfenidone

At baseline, diastolic blood pressure was higher in the pirfenidone 2400 mg group and plasma albumin was higher in the pirfenidone 1200 mg group (Table 1). The diastolic blood pressure was not associated with eGFR change in any treatment group or in the total study population. Multiple linear regression models, however, demonstrated that higher baseline plasma albumin was associated with eGFR change among all groups (p<0.001) (Table 3). Among individual groups, a 0.1 g/dL higher baseline plasma albumin was associated with a significant improvement in eGFR of 0.9 ml/min/1.73 m² in the pirfenidone 2400 group (95% CI 0.1 to 1.7, p=0.03), approached significance in the pirfenidone 1200 group (p=0.06), but was not significant in the placebo group (p=0.21) (Table 3).

Several plasma biomarkers were studied that have correlated with kidney function decline, inflammation or fibrosis in prior studies (Table 4) 13,21-23. Interestingly, the baseline levels of many plasma biomarkers studied were correlated with baseline eGFR (Table 5). The strongest associations were observed with TNF, soluble TNF-R1 (sTNF-R1), and FGF-23, with higher levels associated with lower baseline eGFR. A highly significant correlation between FGF23 and sTNF-R1 (r=0.7273) and TNF (r=0.623), P<0.001 was found for both (Supplemental Table 2). Although the inflammatory plasma biomarkers were highly correlated with the baseline eGFR, none changed significantly with pirfenidone treatment (Supplemental Table 3), and none were statistically significantly associated with eGFR change in any treatment group.

### Adverse Events and Study Non-Completion

The adverse events that contributed to patients' withdrawal from the study are listed in Table 6. Adverse events were predominantly gastrointestinal, fatigue, and photosensitivity rash. None of the adverse effects were significantly more common among the two pirfenidone groups compared to the placebo group, although there were a higher number of subjects who withdrew from the study due to gastrointestinal side effects and fatigue in the pirfenidone groups as compared to placebo. Of note, the incidence of gastrointestinal side effects and fatigue appeared to be dose related. One subject in the high dose pirfenidone group was diagnosed with adenocarcinoma of the prostate within the first few weeks of starting drug and withdrew from the study.

### Discussion

Novel treatment approaches for diabetic nephropathy that reduce the rate of renal function decline are urgently needed, as the number of patients with ESKD attributed to diabetes continues to increase. The available approaches to reduce the rate of renal function decline primarily work in an indirect manner via reducing hyperglycemia or blood pressure.

A rate of decline of eGFR of −2.2±4.8 ml/min per 1.73 m² per year in the placebo group was observed, which is lower than in other recent studies 5,6 demonstrating that conservative therapy was maximized. An improvement in eGFR was observed in the pirfenidone 1200 group with a net increase of +3.3±8.5 ml per min 1.73 m² over the span of 54 weeks. The pirfenidone 2400 group had an intermediate change in eGFR (−1.9±6.7 ml/min per 1.73 m²), which was not significantly different compared to the placebo arm.

The eGFR improvement in the pirfenidone 1200 mg group was noted as early as 6 months after treatment initiation and was maintained through the end-of-study. It is possible that the early increase in eGFR may be due to a hemodynamic effect. Whether the benefit on eGFR is due to a reduction of matrix expansion in the glomerulus or tubulointerstitial compartments remains unknown as biopsies were not performed in this study. The significant improvement in eGFR suggests that treatment to reduce renal fibrosis may confer some degree of regression of the disease process in diabetic nephropathy. Regression of mesangial matrix expansion has been reported in patients with established diabetic nephropathy who underwent a pancreas transplant, based on follow-up biopsies 24,25 however a period of 5-10 years was required for pathologic improvement to be manifest. In animal studies, a combination of ACEi and ARBs have been found to reverse lesions of renal fibrosis 26,27. These studies provide evidence that renal fibrosis is not necessarily irreversible. A potent anti-fibrotic approach may be able to arrest and potentially improve renal function within a short time frame. Future studies with repeated biopsies would be informative to demonstrate whether glomerular and tubulinterstitial markers of fibrosis are reversible with innovative therapies.

Novel non-invasive biomarkers that correlate with progression and/or regression may be suitable as surrogate markers for demonstrating regression and would be of great assistance in future studies evaluating anti-fibrotic therapies. A candidate list of biomarkers within this study was selected. None of the selected biomarkers significantly changed with therapy, or predicted response to therapy. Whether these results reflect low statistical power due to the relatively small sample size within each treatment arm, or whether other markers may reflect treatment response more precisely is presently unknown, and an important area for future research. Of great interest, however, was that several biomarkers were strongly associated with eGFR at baseline. Many of these are considered to be part of the inflammatory response (TNF, sTNF-R1, IFN-γ, and IL-1) and these findings are consistent with recent data in patients with type 1 diabetes 13. As the study included patients with both type 1 and type 2 (predominantly the latter) diabetes, these inflammatory biomarkers validate the results of the prior study and suggest that these factors may signify the ongoing inflammation occurring systemically, and potentially influencing progression within the kidney. Further support for inflammatory mechanisms in diabetic nephropathy is provided from gene expression data and renal biopsies from patients with diabetic nephropathy 14. In these studies using isolated glomeruli, markers of the inflammatory network were strongly up-regulated.

Albuminuria is the classic biomarker for diabetic nephropathy and reduction is thought to be beneficial. A reduction in albuminuria has been found to correlate with reducing the rate of renal function decline with ARB treatment 28. Notably, albuminuria did not decrease with pirfendione treatment. This data is similar to what was observed in a recent open-label clinical study of pirfenidone in patients with advanced FSGS, as pirfenidone treatment was associated with reduction of the rate of renal function decline without attenuating albuminuria 20. Similarly, in an animal model pirfenidone conferred benefit to glomerular histology and reduced gene expression of matrix molecules in the diabetic db/db mouse, without lowering albuminuria 19. Urine levels of TGF-beta have been found to be increased in patients with diabetic nephropathy 29-31 and may reflect ongoing renal production 31. However, in the present study urine levels of TGF-□□ was not significantly affected by pirfenidone; this may be due to the wide variability of urine levels in patients and/or the small sample size evaluated here.

Thus far, no specific marker has been identified to predict the response of pirfenidone. A biomarker that could predict the benefit of pirfenidone and potentially its side effect profile would be of great benefit. In our study, a beneficial effect was noted only with the 1200 mg dose and not 2400 mg. Of note, the dose used in the FSGS open label study was 2400 mg/d (which was tolerated by ⅔ of subjects, with the remainder receiving a lower dose). Of the clinical laboratory variables measured at baseline, the plasma albumin level was significantly associated with change in eGFR in subjects in the pirfenidone 2400 mg group. It is possible that pirfenidone binding to albumin may impact the pharmacokinetic profile, although how increased albumin binding of pirfenidone may be beneficial is unclear. The present study also highlights the need to identify new markers of renal disease progression, apart from albuminuria, that could be used as surrogate markers of anti-fibrotic therapies.

Pirfenidone is a pyridone derivative that has both anti-fibrotic and anti-inflammatory properties. There has been a growing interest in this drug to protect against a variety of progressive fibrotic disorders 32-36. The drug has been approved in Japan for idiopathic pulmonary fibrosis (IPF). Two phase III studies of pirfenidone for IPF in the North America, Europe, and Australia reported equivocal statistical outcomes, but overall reduced disease severity. At present, the drug has not been approved by the FDA for clinical use in the United States.

This study had several important limitations. First, it was designed as an exploratory, small-scale study and the findings may need replication with a larger study. Second, the incomplete ascertainment of outcomes may have influenced the results. In this regard, change in eGFR was the primary outcome, which by definition required eGFR measurements at study entry and end-of-study. Individuals who dropped out were therefore excluded from the efficacy analysis, which may have introduced bias in either direction. Third, the lack of efficacy of high dose pirfenidone remains unexplained.

In conclusion, this study is the first randomized, placebo-controlled clinical trial that demonstrates that an oral anti-fibrotic drug slows renal function decline in subjects with established diabetic nephropathy. The similar effect observed with the use of pirfenidone in focal segmental glomerulosclerosis lends further weight to this observation.

### Concise Methods

### Study Design

The study protocol was approved by the institutional review boards of the participating centers and all patients provided written consent. Trial sites were Thomas Jefferson University, Philadelphia, Pa.; the Mayo Clinic, Rochester, Minn.; and the NIH Clinical Center, Bethesda, Md. At the pre-study evaluation, the potential study candidates provided a medical history and underwent a complete physical examination, including clinical laboratory determinations and the measurement of vital signs. Entry criteria included a history of type 1 or type 2 diabetes, eGFR 20-75 ml/min/1.73 m2, microalbuminuria or overt proteinuria, blood pressure <140/90 mm Hg on an a ACEi, ARB or the combination, if tolerated. Exclusion criteria included other causes of kidney disease, history of photosensitivity rash, and liver disease. A stratified block randomization scheme was used to maximize the balance of patients with type 1 and type 2 diabetes assigned to each treatment group, using randomization blocks of size 4.

Eligible subjects were randomly assigned to receive pirfenidone 1200 mg daily, pirfenidone 2400 mg daily, or placebo for 54 weeks. Subjects received two 400 mg capsules of pirfenidone three times, one capsule of pirfenidone and one capsule of placebo three times daily), or two capsules of placebo three times daily. Subjects who developed nausea, heartburn or reflux, epigastric pain, or severe fatigue that persisted for more than one week had a dose reduction of 25% and then gradually brought back to their intended dose. If symptoms persisted, the dose was kept at the lowest tolerable dose (but not less than 50% of original dose) if the subject desired to continue in the protocol.

### Endpoints

The primary endpoint was eGFR change from baseline to end of study. Baseline eGFR was determined from two serum creatinine measurements within 3 weeks prior to starting study drug. End of study eGFR was determined from two serum creatinine measurements at week 52 and week 54, prior to stopping study medication. Serum creatinine was measured by the Jaffe method separately at each clinic site laboratory. eGFR was determined by the 4-variable Modification of Diet in Renal Disease Study equation 37. Secondary endpoints included the change in urine ACR and the change in urine TGF-□/urine creatinine.

### Biomarker Analysis

Biomarkers were analyzed in plasma and urine from samples collected at both baseline visits and both end-of-study visits, with the two results averaged. Plasma biomarkers were measured by MesoScale Discovery (MSD) platform at the UCSD Clinical Translational Research Institute (CTRI) facility. The MSD platform uses electrochemiluminescence tags on specific antibodies that emit light when electrochemically stimulated. The specific blood biomarkers measured were interferon-y (IFNy), interleukin 1 (IL-1), tumor necrosis factor (TNF), soluble TNF receptor 1 (S TNF R1), YKL40 (also called chitinase like protein or human cartilage glycoprotein-39), brain natriuretic peptide (BNP), and TGF-beta1. Fibroblast growth factor 23 (FGF23) was measured by a C-terminal ELISA assay (Immutopics, San Clemente, Calif.). Urine biomarkers included urine TGF-beta1 (measured by Quantikine, R&D Biosystems, as previously described 29). Urine albumin was measured by nephelometry and urine creatinine was measured by the Jaffe method.

### Statistical Methods

The pre-specified primary endpoint analysis was the eGFR change, compared between each pirfenidone treatment group with placebo group using the Student t-test. Secondary endpoints (ACR change and urine TGF-beta1 change□□s were similarly evaluated. When one of the two visits was missing, a single observation was used. Due to missing data the assumption that the data points arose from an identical distribution (equal variance) was put in question. Therefore, in addition to the two-sample t-tests, we also performed permutation tests with 10,000 permutations of the treatment assignments, as well as an analysis of covariance (ANCOVA) 38 with iterated re-weighted least-squares (IWLS) 39 as confirmatory analyses. Data are presented as mean±SD for normally distributed variables, and medians and interquartile ranges for skewed variables.

In exploratory analysis, linear regression was performed to evaluate associations between baseline eGFR as the dependent variable and each biomarker at baseline as the independent variable. Skewed variables were natural log transformed, and each biomarker was evaluated as “per SD greater” to facilitate comparisons of strengths of association. Biomarker change scores were calculated as (end-of-study value—baseline level). Analysis of variance (ANOVA) was used to determine if the change in biomarker levels differed by randomized treatment assignment. It was also whether baseline biomarker levels were associated with eGFR change using multivariable linear regression. These models were adjusted for the other biomarkers and for clinical factors that are well established markers of progressive diabetic nephropathy, including age, sex, race, SBP, DBP, baseline ACR, and baseline eGFR.

Since some subjects did not complete the study, differences in baseline characteristics were compared between completers and non-completers using the Student t-test or the Mann-Whitney test for continuous variables and chi-squared test or Fisher's exact test for discrete variables. An analysis based on logistic regression models was conducted to determine whether this violates the assumption of missing completely at random (MCAR) 40. This included using baseline characteristics to predict dropout and using eGFR values at a particular visit to predict dropout at the next visit. The latter part of the logistic regression was fitted using generalized estimating equations (GEE) 41. All data analyses were conducted using R and STATA version 11.0 (College Station, Tex.)

### REFERENCES REFERRED TO IN THIS EXAMPLE

1. Steffes, M. W., Osterby, R., Chavers, B. & Mauer, S. M. Mesangial expansion as a central mechanism for loss of kidney function in diabetic patients. Diabetes 38, 1077-1081 (1989).
2. Lane, P., Steffes, M., Fioretto, P. & Mauer, S. Renal interstitial expansion in insulin-dependent diabetes mellitus. Kidney Int 43, 661 (1993).
3. Fioretto, P., Steffes, M., Sutherland, D. & Mauer, M. Sequential renal biopsies in insulin-dependent diabetic patients: Structural factors associated with clinical progression. Kidney International 48, 1929-1935 (1995).
4. Lewis, E., Hunsicker, L., Bain, R. & Rohde, R. The effect of angiotensin-converting-enzyme inhibition on diabetic nephropathy. The New England J Med 329, 1456-1462 (1993).
5. Brenner, B. et al. Effects of losartan on renal and cardiovascular outcomes in patients with Type 2 diabetes and nephropathy. N Engl J Med 345, 861 (2001).
6. Lewis, E. et al. Renoprotective effect of the angiotensin-receptor antagonist irbesartan in patients with nephropathy due to type 2 diabetes. The New England J Med 345, 851-860 (2001).
7. Sharma, K. et al. Captopril-induced reduction of serum levels of transforming growth factor-b 1 correlates with long-term renoprotection in insulin-dependent diabetic patients. Am J Kid Dis 34, 818-823 (1999).
8. Mann, J. F. et al. Renal outcomes with telmisartan, ramipril, or both, in people at high vascular risk (the ONTARGET study): a multicentre, randomised, double-blind, controlled trial. Lancet 372, 547-53 (2008).
9. Sharma, K., Guo, J., Jin, Y. & Ziyadeh, F. N. Neutralization of TGF-b by anti-TGF-b antibody attenuates kidney hypertrophy and the enhanced extracellular matrix gene expression in STZ-induced diabetic mice. Diabetes 45, 522-530 (1996).
10. Ziyadeh, F., Hoffman, B., Han, D., Iglesias-de la Cruz, C., Hong, S., Isono, M., Chen, S., McGowan, T., Sharma, K. Long-term prevention of renal insufficiency excess matrix gene expression and glomerular mesangial matrix expansion by treatment with monoclonal antitransforming growth factor-b antibody in db/db diabetic mice. Proc Natl Acad Sci USA 97, 8015-8020 (2000).
11. Huang, C. et al. Cellular basis of diabetic nephropathy: II. The transforming growth factor-beta system and diabetic nephropathy lesions in type 1 diabetes. Diabetes 51, 3577-81 (2002).
12. Sharma, K. et al. Increased renal production of transforming growth factor-b 1 in patients with type II diabetes. Diabetes 46, 854-859 (1997).
13. Niewczas, M. A. et al. Serum concentrations of markers of TNFalpha and Fas-mediated pathways and renal function in nonproteinuric patients with type 1 diabetes. Clin J Am Soc Nephrol 4, 62-70 (2009).
14. Lorz, C. et al. The death ligand TRAIL in diabetic nephropathy. J Am Soc Nephrol 19, 904-14 (2008).
15. Iyer, S., Gurujeyalakshmi, G. & Giri, S. Effects of pirfenidone on transforming growth factor-B gene expression at the transcriptional level in bleomycin hamster model of lung fibrosis. The Journal of Pharmacology and Experimental Therapeutics 291, 367-373 (1999).
16. Shihab, F., Bennett, W., Yi, H. & Andoh, T. Pirfenidone treatment decreases transforming growth factor-beta1 and matrix proteins and ameliorates fibrosis in chronic cyclosporine nephrotoxicity. American Journal of Transplantation 2, 111-119 (2002).
17. Oku, H., Nakazato, H., Horikawa, T., Tsuruta, Y. & Suzuki, R. Pirfenidone suppresses tumor necrosis factor-alpha, enhances interleukin-10 and protects mice from endotoxic shock. Eur J Pharmacol 446, 167-76 (2002).
18. Grattendick, K. J., Nakashima, J. M., Feng, L., Giri, S. N. & Margolin, S. B. Effects of three anti-TNF-alpha drugs: etanercept, infliximab and pirfenidone on release of TNF-alpha in medium and TNF-alpha associated with the cell in vitro. Int Immunopharmacol 8, 679-87 (2008).
19. RamachandraRao, S. P. et al. Pirfenidone is renoprotective in diabetic kidney disease. J Am Soc Nephrol 20, 1765-75 (2009).
20. Cho, M. E., Smith, D. C., Branton, M. H., Penzak, S. R. & Kopp, J. B. Pirfenidone slows renal function decline in patients with focal segmental glomerulosclerosis. Clin J Am Soc Nephrol 2, 906-13 (2007).
21. Parker, B. D. et al. The associations of fibroblast growth factor 23 and uncarboxylated matrix Gla protein with mortality in coronary artery disease: the Heart and Soul Study. Ann Intern Med 152, 640-8.
22. Marsell, R. et al. Fibroblast growth factor-23 is associated with parathyroid hormone and renal function in a population-based cohort of elderly men. Eur J Endocrinol 158, 125-9 (2008).
23. Larsson, T., Nisbeth, U., Ljunggren, O., Juppner, H. & Jonsson, K. B. Circulating concentration of FGF-23 increases as renal function declines in patients with chronic kidney disease, but does not change in response to variation in phosphate intake in healthy volunteers. Kidney Int 64, 2272-9 (2003).
24. Fioretto, P., Steffes, M. W., Sutherland, D. E. R., Goetz, F. C. & Mauer, M. Reversal of Lesions of Diabetic Nephropathy after Pancreas Transplantation. N Engl J Med 339, 69-75 (1998).
25. Fioretto, P., Kim, Y. & Mauer, M. Diabetic nephropathy as a model of reversibility of established renal lesions. Curr Opin Nephrol Hypertens 7, 489-94 (1998).
26. Ma, L. J. et al. Regression of sclerosis in aging by an angiotensin inhibition-induced decrease in PAI-1. Kidney Int 58, 2425-36 (2000).
27. Ma, L.-J. & Fogo, A. Role of angiotensin II in glomerular injury. Seminars in Nephrology 21, 544-553 (2001).
28. Eijkelkamp, W. B. et al. Albuminuria is a target for renoprotective therapy independent from blood pressure in patients with type 2 diabetic nephropathy: post hoc analysis from the Reduction of Endpoints in NIDDM with the Angiotensin II Antagonist Losartan (RENAAL) trial. J Am Soc Nephrol 18, 1540-6 (2007).
29. Agarwal, R., Siva, S., Dunn, S. R. & Sharma, K. Add-On Angiotensin II Receptor Blockade Lowers Urinary Transforming Growth Factor-{beta} Levels. American Journal of Kidney Diseases 39, 486-492 (2002).
30. McGowan, T. A., Dunn, S. R., Falkner, B. & Sharma, K. Stimulation of Urinary TGF-{beta} and Isoprostanes in Response to Hyperglycemia in Humans. Clin J Am Soc Nephrol 1, 263-268 (2006).
31. Sharma, K. et al. Increased renal production of transforming growth factor-beta1 in patients with type II diabetes. Diabetes 46, 854-859 (1997).
32. Gahl, W. et al. Effect of pirfenidone on the pulmonary fibrosis of Hermansky-Pudlak syndrome. Mol Genetics & Metab 76, 234-242 (2002).
33. Bowen, J., Maravilla, K. & Margolin, S. Open-label study of pirfenidone In patients with progressive forms of multiple sclerosis. Multiple Sclerosis 9, 280-283 (2003).
34. Park, H. et al. Pirfenidone suppressed the developoment of glomerulosclerosis in the FGS/Kist mouse. Journal of Korean Medical Science 18, 527-533 (2003).
35. Giri, S. et al. Amelioration of doxorubicin-induced cardiac and renal toxicity by pirfenidone in rats. Cancer Chemotherapy & Pharmacology 53, 141-150 (2004).
36. Tada, S. et al. Pirfenidone inhibits dimethylnitrosamine-induced hepatic fibrosis in rats. Clin & Exp Pharmacology and Physiology 28, 522-527 (2001).
37. Levey, A. S. et al. A more accurate method to estimate glomerular filtration rate from serum creatinine: A new prediction equation. Ann Int Med 130, 461-470 (1999).
38. Yang L, T. A. Efficiency study of estimators for a treatment effect in a pretest-posttest trial. The American Statistician 55, 314 (2001).
39. Venables, W. N., Ripley, B. D. Modern Applied Statistics with S, (Springer, 2002).
40. Ridout, M. S. Testing for random dropouts in repeated measurement data. Biometrics 47, 1617-9; discussion 1619-21 (1991).
41. Zeger, S. L. & Liang, K. Y. Longitudinal data analysis for discrete and continuous outcomes. Biometrics 42, 121-30 (1986).

TABLE 1
Characteristics of Study Participants at Study Entry

| | | Prifenidone | Prifenidone | |
|---|---|---|---|---|
| | Placebo | 1200 | 2400 | P-value |
| n | 21 | 17 | 14 | |
| Age, mean ± SD | 59 ± 12 | 58 ± 10 | 55 ± 13 | 0.56 |
| Male n, % | 12 (57%) | 11 (65%) | 10 (71%) | 0.69 |
| Black n, % | 6 (29%) | 7 (41%) | 2 (21%) | 0.48 |
| Weight (lbs), mean ± SD | 210 ± 50 | 206 ± 28 | 218 ± 49 | 0.74 |
| BMI (kg/m²⁾, mean ± SD | 31.9 ± 7.2 | 31.5 ± 5.3 | 32.8 ± 8.2 | 0.88 |
| SBP (mmHg) mean ± SD | 129 ± 15 | 123 ± 9 | 130 ± 10 | 0.24 |
| DBP (mmHg) mean ± SD | 71 ± 9 | 70 ± 8 | 78 ± 7 | 0.02 |
| Heart Rate (bpm), mean ± SD | 71 ± 8 | 70 ± 12 | 71 ± 13 | 0.94 |
| Diabetes Mellitus type 2 n, % | 16 (76%) | 13 (76%) | 11 (79%) | 0.99 |
| Diabetes duration (years), mean ± SD | 24 ± 13 | 18 ± 10 | 19 ± 9 | 0.25 |
| ACE/ARB use n, % | | | | 0.55 |
| ACE only | 6 (29%) | 7 (41%) | 4 (29%) | |
| ARB only | 10 (48%) | 3 (18%) | 5 (36%) | |
| ACE + ARB | 4 (19%) | 6 (35%) | 5 (36%) | |
| Smoking n, % | | | | 0.68 |
| Current | 1 (5%) | 0 (0%) | 0 (0%) | |
| Former | 11 (52%) | 8 (50%) | 5 (38%) | |
| HbA1c (g/dL), mean ± SD | 7.3 ± 1.4 | 7.4 ± 1.2 | 7.1 ± 1.4 | 0.81 |
| HDL cholesterol (mg/dL), mean ± SD | 45 ± 11 | 44 ± 9 | 43 ± 12 | 0.86 |
| LDL cholesterol (mg/dL), mean ± SD | 96 ± 28 | 108 ± 39 | 104 ± 24 | 0.53 |
| Triglycerides (mg/dL), median (IQR)* | 104 (70, 171) | 116 (76, 188) | 109 (88, 157) | 0.83 |
| Serum albumin (g/dl) mean ± SD | 4.4 ± 0.3 | 4.6 ± 0.4 | 4.2 ± 0.4 | 0.04 |
| eGFR (ml/min/1.73 m²), mean ± SD | 39 ± 13 | 38 ± 13 | 39 ± 13 | 0.95 |
| Urine albumin/creatinine (mg/g) | 79 (36, 514) | 131 (48, 450) | 252 (118, 984) | 0.38 |
| median (IQR)* | | | | |

Legend. Baseline demographic, clinical and laboratory parameters for study completers are presented. Data are presented as mean ± SD, with analysis by Chi Square or Fisher's Exact tests for categorical variables and ANOVA for continuous variables, except for variables (*) for which data was not normally distributed which are presented as median and IQR and were analyzed by Kruskal-Wallis test.

TABLE 2
Change in eGFR Over 54 Weeks by Treatment Group

| | | eGFR Change, |
|---|---|---|
| | eGFR Change | with imputation |
| | (ml/min/1.73 m²) | (ml/min/1.73 m²) |
| Placebo | −2.2 ± 4.8 | −3.7 ± 5.7 |
| Prifenidone 1200 mg | 3.3 ± 8.5* | 3.3 ± 8.5** |
| Prifenidone 2400 mg | −1.9 ± 6.7 | −2.1 ± 6.4 |
| Total | −0.3 ± 7.0 | −0.7 ± 9.1 |

Legend. Shown are the eGFR changes over the course of the study in the 52 study completers and in the 52 completers plus the 5 subjects who began dialysis during the study, for whom an end-of-study eGFR was imputed as 10 ml/min per 1.73 m2.
*denotes p = 0.026 versus placebo and
**denotes p < 0.006 versus placebo.

TABLE 3
Association of Baseline Serum Albumin (per
0.1 g/L higher) with 54 Week eGFR Change.

| | N | eGFR Change (95% CI) | P-value |
|---|---|---|---|
| All Completers | 52 | 0.9 (0.5, 1.4) | <0.001 |
| Placebo Arm | 21 | 0.4 (−0.2, 1.1) | 0.21 |
| Prifenidone 1200 | 17 | 1.1 (0.0, 2.2) | 0.06 |
| Prifenidone 2400 | 14 | 0.9 (0.1, 1.7) | 0.03 |

Legend. Shown is the association of baseline serum albumin on change in eGFR over the 54 week period, using linear regression. Positive values indicate that a higher serum albumin level was associated with improvement in eGFR during the course of the study.

TABLE 4
Association of baseline plasma biomarker
levels with baseline eGFR in completers.

| Biomarker | N | Change GFR (95% CI) | P |
|---|---|---|---|
| IFN-gamma * | 48 | −5.1 (−8.7, −1.5) | 0.006 |
| IL-1 * | 48 | −4.4 (−8.1, −0.8) | 0.019 |
| TNF | 48 | −6.7 (−10.0, −3.3) | <0.001 |
| sTNF-R1 | 47 | −9.0 (−11.9, −6.2) | <0.001 |
| FGF-23 * | 48 | −8.1 (−11.2, −5.0) | <0.001 |
| YKL-40 | 47 | −4.8 (−8.5, −1.1) | 0.011 |
| BNP * | 46 | −0.9 (−4.8, 3.1) | 0.667 |

Legend. Shown are the cross-sectional associations of biomarkers at the baseline study visit with eGFR at the baseline study visit using linear regression. Skewed variables were natural logarithm transformed (*), subsequently, each biomarker modeled as per 1 SD higher, to facilitate comparison in strengths of association. Negative values indicate that higher baseline biomarker level was associated with a lower GFR at the baseline study visit.

TABLE 5
Reasons for subject non-completion

| | | Prifenidone | Prifenidone | |
|---|---|---|---|---|
| | Placebo | 1200 mg | 2400 mg | |
| | (n = 27) | (n = 26) | (n = 25) | P-value |
| N | 6 | 9 | 11 | |
| Dialysis | 4 (15%) | 0 (0%) | 1 (4%) | 0.12 |
| Gastrointestinal | 1 (4%) | 3 (13%) | 6 (25%) | 0.10 |
| symptoms | | | | |
| Rash | 0 (0%) | 1 (4%) | 1 (4%) | 0.53 |
| Heart failure | 1 (4%) | 1 (4%) | 0 (0%) | 0.77 |
| Aortic dilation | 0 (0%) | 1 (4%) | 0 (0%) | 0.32 |
| Urinary tract | 0 (0%) | 0 (0%) | 1 (4%) | 0.64 |
| infection | | | | |
| Fatigue | 0 (0%) | 0 (0%) | 3 (12%) | 0.06 |
| Cancer | 0 (0%) | 0 (0%) | 1 (4%) | 0.65 |
| Lost to follow up | 0 | 1 | 0 | |

Legend. Shown are the number of subjects randomized to each treatment group who discontinued treatment before end-of-study, together with the reasons for discontinuation. Several subjects had more than one adverse event that contributed to their withdrawal from the study. 1 patient withdrew from study for personal reasons before the 1^{st} post-treatment visit. P value was calculated using Pearson's Chi-square test.

SUPPLEMENTAL TABLE 1
Demographic and baseline laboratory differences
between study completers and non-completers

| | Completers | Non-Completers | P- |
|---|---|---|---|
| | (n = 52) | (n = 26) | value |
| Randomization, n (%) | | | 0.25 |
| Placebo | 21 (78%) | 6 (22%) | |
| Prifenidone 1200 | 17 (65%) | 9 (35%) | |
| Prifenidone 2400 | 14 (56%) | 11 (44%) | |
| Age (yrs) ± SD | 58 ± 12 | 59 ± 18 | 0.75 |
| Male n, (%) | 33 (63%) | 16 (62%) | 0.87 |
| Black n, % | 16 (31%) | 10 (38%) | 0.50 |
| eGFR ml/min/1.73 | 38 ± 13 | 35 ± 15 | 0.32 |
| m² ± SD | | | |
| ACR mg/g, Median (IQR)* | 143 (43, 514) | 226 (105, 1198) | 0.16 |
| Urine TGFβ pg/mg | 4.8 (2.8, 7.3) | 5.2 (1.5, 10.1) | 0.84 |
| creat, Median (IQR)* | | | |

Legend. Demographics and baseline laboratory differences between study completers and non-completers are shown. Differences were assessed by Student t test or Mann-Whitney rank sum test (*) for continuous variables, and the Chi Square test or Fisher's Exact test for categorical variables.

SUPPLEMENTAL TABLE 2
Baseline Biomarker Values

| | | Prifenidone | Prifenidone | |
|---|---|---|---|---|
| Biomarker | Placebo | 1200 mg | 2400 mg | P-value |
| N | 19 | 17 | 12 | |
| Urine TGF-β/creatinine | 4.6 (2.5, 7.1) | 5.4 (3.2, 7.1) | 4.2 (2.4, 12.3) | 0.90 |
| (pg/mg), median (IQR) | | | | |
| Plasma TGF-β (ng/ml), | 7.8 (4.0, 43.8) | 4.2 (2.4, 7.1) | 4.7 (3.5, 9.0) | 0.16 |
| median (IQR) | | | | |
| Serum brain naturetic | 261 (150, 421) | 318 (234, 400) | 237 (160, 327) | 0.49 |
| peptide (pg/ml), mean ± | | | | |
| SD | | | | |
| Serum fibroblast growth | 130 (82, 335) | 116 (68, 216) | 103 (53, 182) | 0.21 |
| factor 23 (RU/ml), | | | | |
| median (IQR) | | | | |
| Serum interferon-gamma | 0.40 (0.05, 1.34) | 0.64 (0.05, 1.03) | 0.46 (0.18, 1.76) | 0.75 |
| (pg/ml), median (IQR) | | | | |
| Serum interleukin 1β | 0.23 (0.05, 0.33) | 0.05 (0.05, 1.18) | 0.20 (0.05, 0.30) | 0.19 |
| (pg/ml), mean ± SD | | | | |
| Serum tumor necrosis | 8.9 ± 3.2 | 7.7 ± 2.6 | 8.4 ± 3.7 | 0.53 |
| factor (pg/ml), mean ± | | | | |
| SD | | | | |
| Serum soluble TNF | 2.9 (2.4, 4.2) | 2.8 (2.3, 3.9) | 2.6 (2.4, 3.9) | 0.67 |
| receptor 1 (ng/ml), | | | | |
| median (IQR) | | | | |
| Serum YKL-40 (ng/ml), | 138 ± 86 | 122 ± 76 | 130 ± 111 | 0.87 |
| mean ± SD | | | | |

Legend. Shown are baseline biomarker data for study completers only. Data for peptide factors that were normally distributed are presented as mean ± SD and were analyzed by ANOVA, while data that were not normally distributed are presented as median and interquartile range (IQR) and analyzed by the Kruskal Wallis test.

SUPPLEMENTAL TABLE 3
Biomarker correlation matrix.

| | | | | | | | | Ln | |
|---|---|---|---|---|---|---|---|---|---|
| | Ln | Ln(IL- | Ln (TNF | S TNF | Ln(FGF- | YKL- | | (Plasma | Baseline |
| | (IFN) | 1) | α) | R1 | 23) | 40 | Ln(BNP) | TGF) | eGFR |
| Ln (IFN) | 1 | | | | | | | | |
| Ln(IL-1) | 0.3232 | 1 | | | | | | | |
| Ln (TNF α) | 0.398 | 0.2538 | 1 | | | | | | |
| S TNF R1 | 0.1775 | 0.4776 | 0.6264 | 1 | | | | | |
| Ln(FGF-23) | 0.2327 | 0.3068 | 0.6392 | 0.7273 | 1 | | | | |
| YKL-40 | 0.1936 | 0.2724 | 0.2559 | 0.4319 | 0.3329 | 1 | | | |
| Ln(BNP) | −0.0687 | 0.0073 | −0.0079 | −0.0021 | −0.1285 | −0.0865 | 1 | | |
| Ln(Plasma | −0.0521 | 0.3618 | 0.0385 | −0.0745 | 0.0274 | −0.0337 | −0.4002 | 1 | |
| TGF) | | | | | | | | | |
| Baseline | −0.3904 | −0.338 | −0.5092 | −0.6870 | −0.6184 | −0.3659 | −0.0652 | 0.273 | 1 |
| GFR | | | | | | | | | |

Legend: Correlation matrix of baseline biomarkers with one another and with baseline eGFR. Skewed variables were natural logarithm transformed (*), and correlations (r) were derived using Pearson's pairwise correlations.

SUPPLEMENTAL TABLE 3
Changes in biomarkers over 52 weeks compared across study groups

| | Placebo | Prifenidone 1200 | Prifenidone 2400 | |
|---|---|---|---|---|
| | n = 18 | n = 14 | n = 14 | P-value |
| Change in IFN gamma (pg/ml) | 0.3 ± 1.5 | 0.1 ± 1.9 | 0.2 ± 2.5 | 0.97 |
| Change in IL1 (pg/ml)* | −0.1 (−0.3, 0.1) | 0.0 (−0.3, 0.4) | 0.0 (−0.2, 0.2) | 0.39 |
| Change in TNF α (pg/ml) | −0.6 ± 2.4 | −0.8 ± 2.2 | −0.7 ± 2.1 | 0.99 |
| Change in S TNF R1 (pg/ml)* | 177 (−190, 431) | −140 (−497, 148) | 197 (−113, 420) | 0.27 |
| Change in FGF23 (RU/ml)* | 22 (−23, 40) | −3 (−23, 33) | 13 (−6, 63) | 0.55 |
| Change in YKL40 (ng/ml) | 16 ± 61 | 6 ± 62 | 30 ± 43 | 0.55 |
| Change in BNP (pg/ml)* | −13 (−216, 107) | −22 (−114, 24) | 45 (−67, 312) | 0.23 |
| Change in Plasma TGF Beta | | | | |
| (pg/ml) | −12 ± 25 | −2 ± 26 | 7 ± 17 | 0.10 |

Legend. Changes in biomarkers from baseline to end-of-study are presented as mean ± SD and compared among treatment groups by ANOVA; data that was not normally distributed are presented as median and IQR and were compared by the Kruskal-Wallis test (*).

SUPPLEMENTAL TABLE 3
Changes in biomarkers over 52 weeks compared across study groups

| | Placebo | Prifenidone 1200 | Prifenidone 2400 | |
|---|---|---|---|---|
| | n = 18 | n = 14 | n = 14 | P-value |
| Change in IFN gamma (pg/ml) | 0.3 ± 1.5 | 0.1 ± 1.9 | 0.2 ± 2.5 | 0.97 |
| Change in IL1 (pg/ml)* | −0.1 (−0.3, 0.1) | 0.0 (−0.3, 0.4) | 0.0 (−0.2, 0.2) | 0.39 |
| Change in TNF α (pg/ml) | −0.6 ± 2.4 | −0.8 ± 2.2 | −0.7 ± 2.1 | 0.99 |
| Change in S TNF R1 (pg/ml)* | 177 (−190, 431) | −140 (−497, 148) | 197 (−113, 420) | 0.27 |
| Change in FGF23 (RU/ml)* | 22 (−23, 40) | −3 (−23, 33) | 13 (−6, 63) | 0.55 |
| Change in YKL40 (ng/ml) | 16 ± 61 | 6 ± 62 | 30 ± 43 | 0.55 |
| Change in BNP (pg/ml)* | −13 (−216, 107) | −22 (−114, 24) | 45 (−67, 312) | 0.23 |
| Change in Plasma TGF Beta | −12 ± 25 | −2 ± 26 | 7 ± 17 | 0.10 |
| (pg/ml) | | | | |

Legend. Changes in biomarkers from baseline to end-of-study are presented as mean ± SD and compared among treatment groups by ANOVA; data that was not normally distributed are presented as median and IQR and were compared by the Kruskal-Wallis test (*).

Based on this study, the basis for why pirfenidone benefited some patients and not others was unclear. Despite available clinical data and protein biomarker studies there was little understanding on how to dose and prescribe this drug in a future study. Urine metabolomics was performed in subjects enrolled in this study at baseline and at 6 months after enrollment. A panel of metabolites based on a specific ratio added predictive value to the outcome on the drug. Additionally, two metabolites (azelaic acid and citric acid) were affected by the drug and correlated with outcomes. Similar approaches can be employed with other clinical trials to identify which patients will respond to drug, which patients will not, and identify potential new mechanisms. Additionally, metabolite profiles could predict adverse events in patients that are ongoing or will develop in the future.

### Example 3

### Prediction of Medical Complications in Patients with Diabetes

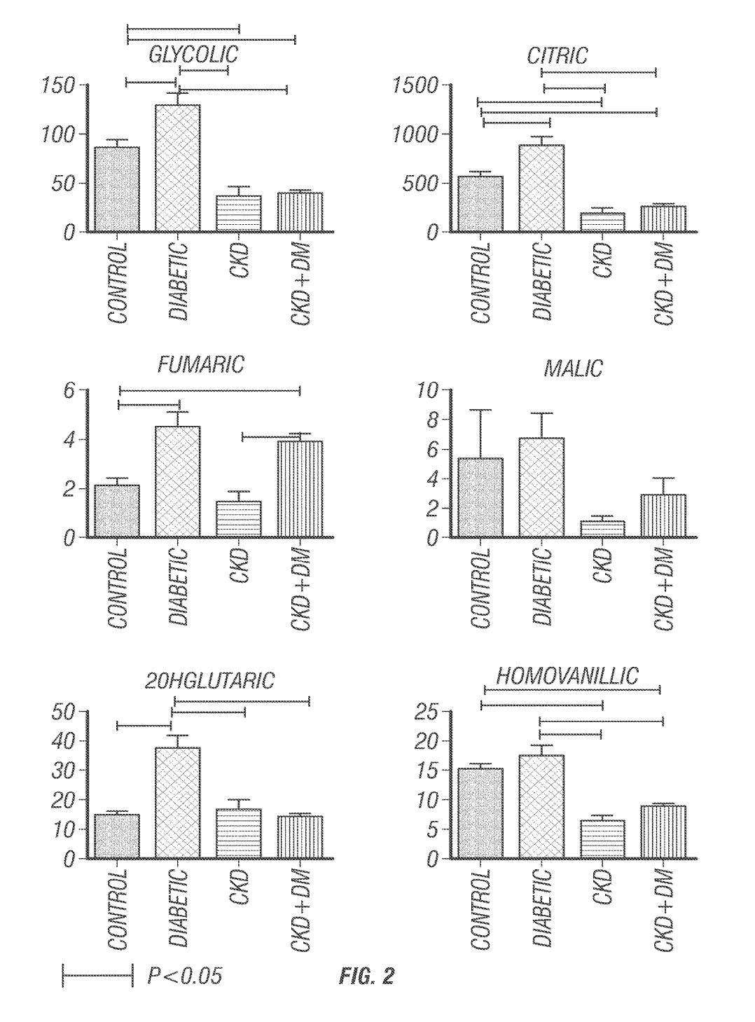

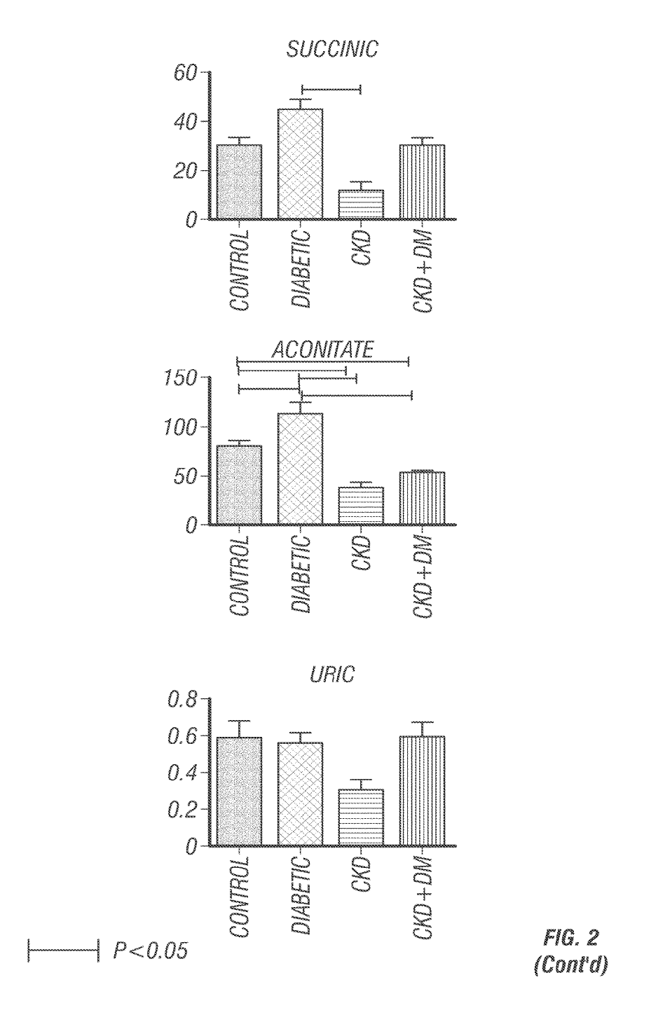

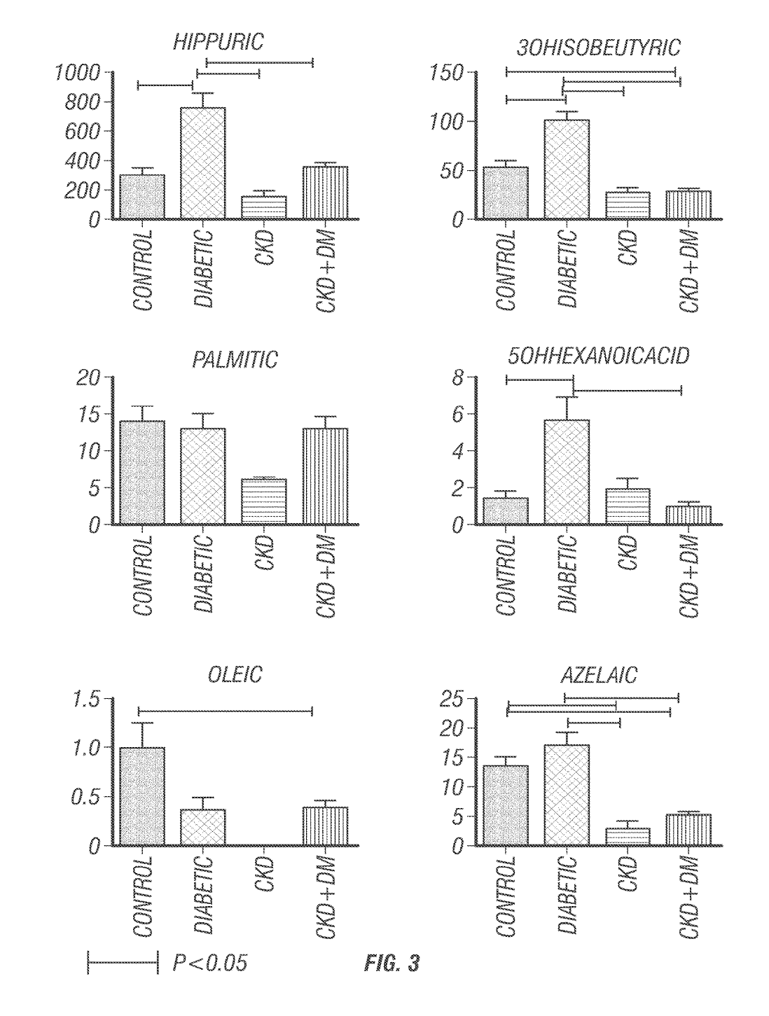

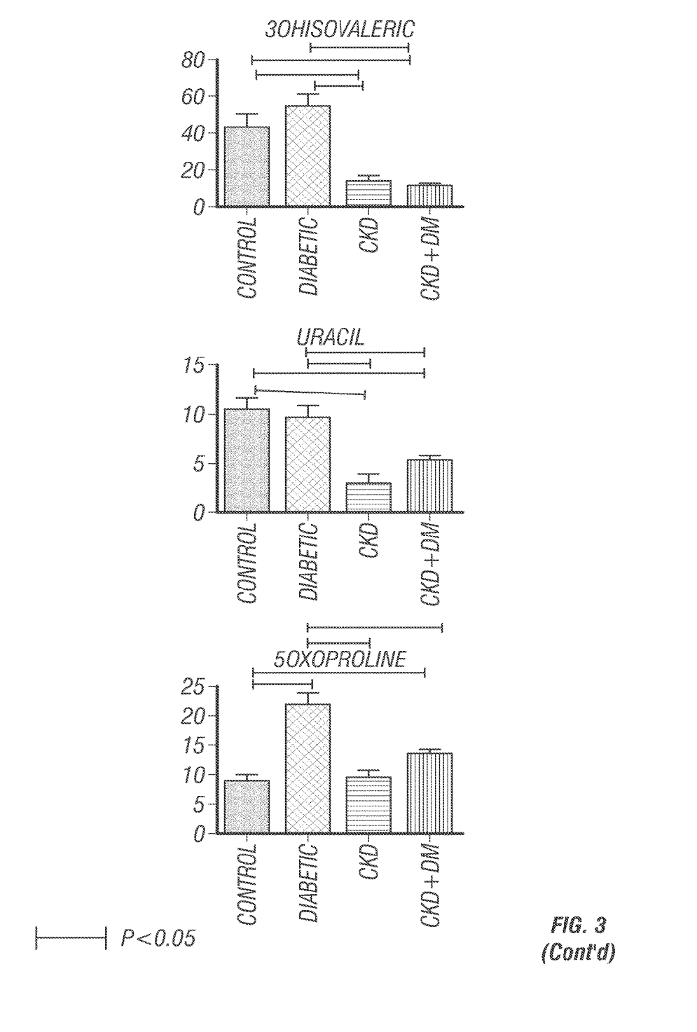

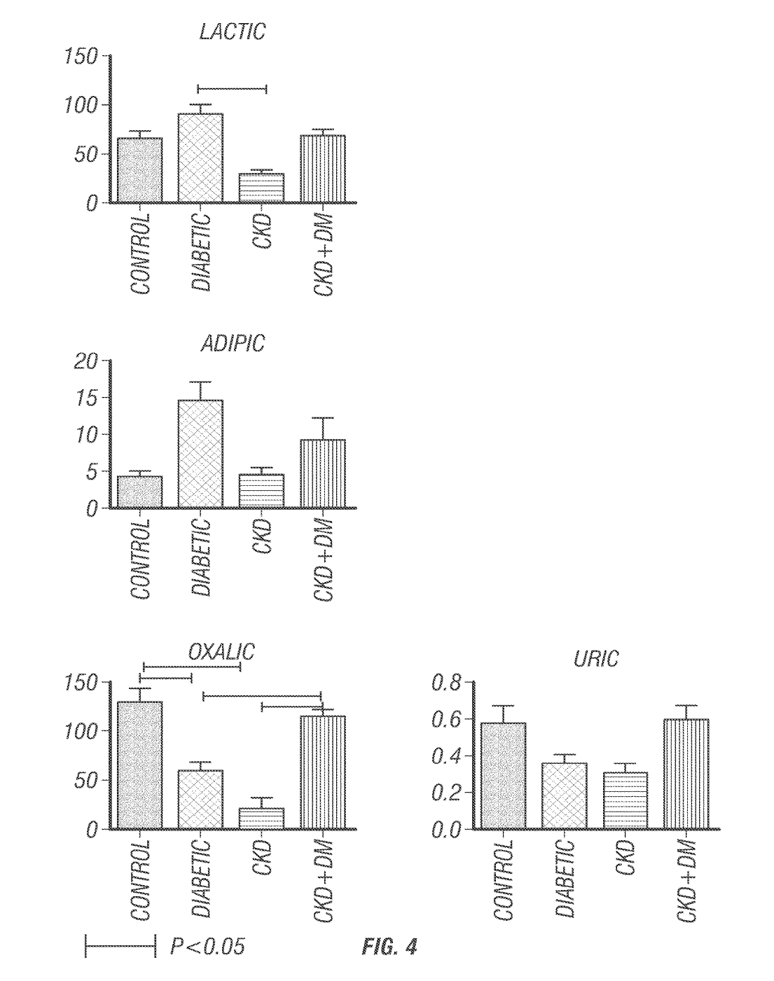

Many patients with diabetes do not develop diabetes-related complications, such as microvascular complications (for example, nephropathy, neuropathy, retinopathy) or macrovascular complications (for example, cardiovascular, peripheral vascular or cerberovascular disease). Often, once they develop evidence of kidney disease, their risk for these other complications increases. Using the methods provided herein, patients having or at risk for kidney disease may be identified before the onset of severe kidney disease. Those patients identified as having or being at risk for kidney disease may then be identified as being likely to develop of microvascular and macrovascular complications. Also, the methods provided herein for identifying kidney disease may also be used to identify patients at risk for developing diabetes-related complications, such as microvascular or macrovascular complications. The patients are identified determined based on the levels of metabolites such as, for example, lactic acid, glycolic acid, fumaric acid, malic acid, adipic acid, 2-OH-glutaric acid, aconitic acid, homovanillic acid, stearic acid, 3-OH-isobutyric acid, palmitic acid, and citrate found in urine or blood samples from the subject. (FIGS. 2-4).

### Example 4

### Prediction of Diabetes, Cardiovascular Disease, Hypertension, and CKD in Individuals with Obesity

Many subjects are obese due to an imbalance in caloric intake and expenditure. However, most of these subjects do not develop medical complications (hypertension, CKD, Cardiovascular disease) or diabetes. A group of human subjects who are overweight (BMI>recommended for that ethnic group) are asked to contribute simultaneous urine samples. After normalizing urine sample density, a urine metabolite profile is collected as detailed herein. A profile including lactic acid, glycolic acid, fumaric acid, malic acid, adipic acid, 2-OH-glutaric acid, aconitic acid, homovanillic acid, stearic acid, 3-OH-isobutyric acid, palmitic acid, and citrate found in urine or blood samples from the subject predicts underlying organ dysfunction that portends diabetes, cardiovascular disease, hypertension or CKD in specific individuals of asymptomatic members of the group.

### Example 5

### Prediction of Diabetes, Cardiovascular Disease, Hypertension, and CKD in Individuals with Obesity

The procedure of Example 4 is repeated with a blood sample collected in concert with the urine sample. The blood sample trends parallel those for urine samples when compared to blood sample controls.

### Example 6

### Prediction of Hypertension and Complications of Hypertension

High blood pressure is found in many individuals and puts patients at risk of cardiovascular and kidney complications. Through the measurement of lactic acid, glycolic acid, fumaric acid, malic acid, adipic acid, 2-OH-glutaric acid, aconitic acid, homovanillic acid, stearic acid, 3-OH-isobutyric acid, palmitic acid, and citric acid found in urine or blood samples from the subject, one can identify subtle signs of organ dysfunction in patients with hypertension and be able to monitor organ dysfunction in response to drug or non-drug therapies.

### Example 7

### Ancillary Liver Involvement

Many individuals with obesity, diabetes or CKD have liver disease. The methods provided herein provide an easier method of detecting liver disease through measurement of lactic acid, glycolic acid, fumaric acid, malic acid, adipic acid, 2-OH-glutaric acid, aconitic acid, homovanillic acid, stearic acid, 3-OH-isobutyric acid, palmitic acid, and citric acid found in urine or blood samples from the subject. By reviewing the metabolic profile of the subject, liver function is determined. The results may provide sensitive clues as to underlying liver function and its response to drug and non-drug therapies, such as diet, exercise, or lifestyle changes through repeated testing of samples as function of time, alone or in combination with conventional liver enzyme measurements. Other metabolites in the urine may also provide informational value to understand the development and severity of liver disease.

### Example 8

### Ancillary Joint Involvement

Many individuals with obesity, diabetes or CKD have joint involvement, such as arthritis or osteoarthritis. The methods provide herein provide an easier method of detecting joint involvement. Using the methods provided herein, through measurement of lactic acid, glycolic acid, fumaric acid, malic acid, adipic acid, 2-OH-glutaric acid, aconitic acid, homovanillic acid, stearic acid, 3-OH-isobutyric acid, palmitic acid, citric acid, and 5-oxoproline found in urine or blood samples from the subject, joint involvement is determined. The results provide sensitive clues as to underlying joint involvement and its response to drug and non-drug therapies, such as diet, exercise, or lifestyle changes through repeated testing of samples as function of time, alone or in combination with conventional ultrasound and MM measurements of joints. Other metabolites in the urine may also provide informational value to understand the development and severity of joint disease.

### Example 9

### Ancillary Lung Disease

Many individuals with obesity, diabetes or CKD have lung involvement, such as sleep apnea, restrictive lung disease or obstructive lung disease. Through measurement of lactic acid, glycolic acid, fumaric acid, malic acid, adipic acid, 2-OH-glutaric acid, aconitic acid, homovanillic acid, stearic acid, 3-OH-isobutyric acid, palmitic acid, citric acid, and 5-oxoproline found in urine or blood samples from the subject, using the methods provided herein, lung involvement is determined. The results provide sensitive clues as to response to drug and non-drug therapies, such as diet, exercise, or lifestyle changes through repeated testing of samples as function of time, alone or in combination with conventional pulmonary measurements. Other metabolites in the urine may also provide informational value to understand the development and severity of lung disease.

### Example 10

### Urine Metabolomics Reveals a Signature of Mitochondrial Dysfunction in Diabetic Kidney DiseaseClinical Trial of the Therapeutic Pirfenidone for Diabetic Nephropathy

### Summary

### Background

Diabetic kidney disease is the leading cause of end stage kidney disease. In the present study, we show that urine metabolomic analysis can be used to identify a metabolic signature of diabetic kidney disease.

Methods:

We employed gas chromatography-mass spectrometry to quantify 94 urine metabolites in 2 independent groups of patients with diabetes and reduced estimated glomerular filtration rate (eGFR) (screening [n=24] and validation groups [n=82], respectively), in patients with type 1 diabetes and normal eGFR (n=27), in patients with type 2 diabetes and normal eGFR (n=25), and in healthy controls (n=23).

### Results

Seventeen metabolites were found to be significantly different in the patients with diabetes and kidney disease vs. controls in the screening group, using a false discovery threshold of p<0.007. Thirteen of these metabolites were confirmed to be different in the independent validation cohort. Urine concentrations of all 13 metabolites were reduced in patients with diabetic kidney disease compared to controls. Network analysis identified that the majority of the metabolites ( 12/13) were linked to mitochondrial metabolism and suggested suppression of mitochondrial activity. To independently test this hypothesis, we found a reduction in kidney levels of cytochrome oxidase subunit II in tissue sections, reduced mitochondrial DNA in urine exosomes, and a reduction in PGClalpha from kidney tissues from patients with diabetic kidney disease.

### Conclusions

We conclude that urine metabolomics is a rich source of biomarkers for diabetic complications, and that, based on the metabolomic signature, diabetic kidney disease is a state of marked reduction of mitochondrial function.

References to tables in this Example refer to tables in Example 10.

### Results and Discussion

### Introduction

Diabetic kidney disease continues to increase worldwide without much evidence of abating 1. Apart from driving increasing rates of end stage kidney disease, progression of kidney disease is a major sign of overall cardiovascular disease and all cause mortality in patients with diabetes 2. The basis of the progression of diabetic kidney disease remains unknown despite numerous investigations using genomics, transcriptomics and proteomics 3-5.

Metabolomics is a systematic evaluation of small molecules and may provide fundamental biochemical insights into disease pathways. Prior studies have evaluated plasma metabolomics in diabetes and end stage kidney disease and revealed alterations in branched chain and aromatic amino acid metabolism and accumulation of metabolites 6. However, with current methods, plasma presents a narrow subset of compounds related to intermediary metabolism. In contrast, urine metabolomics offers a wider range of measurable metabolites 7-9 as the kidney is responsible for concentrating a variety of metabolites and excreting them in the urine. In addition, urine metabolomics may offer direct insights into biochemical pathways linked to kidney dysfunction. However, the relationship of urine metabolomics with diabetic kidney disease has not been comprehensively evaluated. To that end, in the present study we evaluated cohorts of patients with diabetes with and without overt kidney disease with a rigorous quantitative urine metabolomics approach. The studies reveal a characteristic signature for diabetic kidney disease.

### Methods

### Study Populations

Five separate clinical groups were obtained for analysis. A control group of healthy subjects (n=23) and a screening group of 24 consecutive patients with type 2 diabetes and the presence of chronic kidney disease (eGFR<60 ml/min/1.73 m2) were enrolled from the San Diego region. The screening group provided 24-hour urine collections during the same time interval as the control group. An independent validation group was comprised of 82 patients who had a history of diabetes, reduced eGFR and also had undergone 24 h urine collections. The patients in the validation group included patients with either type 1 or type 2 diabetes with kidney disease from various geographic locations within the United States (enrolled from the Pirfenidone Study 10) and from Finland (as part of the FinnDiane Study) 2. Additional cohorts included patients from the FinnDiane Study with type 1 diabetes (n=27) and patients with type 2 diabetes (n=25) who had an eGFR>75 ml/min/1.73 m2 at the time of 24 h urine collection. All groups had urine aliquots stored at <−70° C. for a period of 3 months to 6 years prior to analysis. The institutional review board of the University of California, San Diego and Helsinki University Central Hospital, Finland approved the study and written informed consent was obtained from all the subjects.

### Urine Metabolomics

Aliquots of the frozen 24 h urine collection were thawed and analyzed for creatinine content before being processed for analysis at the UCSD Biochemical Genetics and Metabolomics Laboratory. Samples underwent oximation of ketoacids with pentafluorobenzylhydroxylamine, lyophilization, isolation of organic acids by liquid partition chromatography on silica (42% 2-methyl-2-butanol in chloroform), evaporation of the eluate, and silylation of the dry residue with Trisil-N,O-bis (trimethylsilyl) trifluoroacetamide 11. Aliquots corresponding to 1 μmol of creatinine were applied by injection onto a 30 m×0.32 mm column (Agilent DB-5) in a gas chromatogram (Agilent 5890), eluted with a 4° C./min gradient of 70 to 300° C., and analytes were detected by electron impact mass spectrometry (Agilent 5973 mass selective detector). Each compound was identified by spectrum and confirmed ratio of a qualifying ion and quantifying ion. The quantifying ion's integrated current was used to estimate concentration based upon standard curves for targeted metabolites or based on ratio to 4-nitrophenol or the oximated derivative of 2-ketocaproic acid. As per our procedure, approved by the College of American Pathologists, we maintain 4 to 6 point calibration curves on 83 adducts of 76 compounds; for other compounds (e.g. when authentic standards are not available) concentrations are estimated relative to the quantity of the appropriate internal standard (4-nitrophenol or 2-oxocaproate). The results are reported in μmol organic acid per mmol creatinine.

### Biochemical and Protein-Metabolite Network

We took the 13 metabolites significant for diabetic kidney disease and searched for them using the global map of human metabolic pathway in the KEGG database (http://www.genome.jp/kegg/pathway/map/map01100.html). Of the metabolites we searched, only seven could be mapped to the pathway. For these, we were able to identify their associated enzymes. For the rest, we manually listed the enzymes based on consensus knowledge from the UCSD Biochemical Genetics Lab's internal database. We then downloaded human protein-protein interactions (PPIs) from the BioGrid database (http://thebiogrid.org/) and searched for interactions involving the known enzymes. Finally, the interaction network was constructed and visualized through Cytoscape (http://www.cytoscape.org/).

### Kidney Sections

Unstained slides of kidney samples from biopsy specimens diagnosed as diabetic nephropathy (n=5) or normal (n=5) kidneys were obtained from Dr. Agnes Fogo of Vanderbilt University. These samples were exempt from the requirement of informed consent, according to the institutional review board's approved use of organs and tissues from deceased donors for research. Unstained sections were processed for immunostaining using standard protocols. Sections were incubated first with mouse anti-cytochrome C oxidase, subunit II (Abcam) primary antibody and subsequently with biotin-conjugated α-mouse secondary antibody (Santa Cruz Biotechnology) and HRP-streptavidin (BD Biosciences). Labeling was visualized with chromogen diaminobenzidine (DAB) (Vector Labs, SK-4100), and sections were counterstained with hematoxylin. Sections were digitally scanned at 20× magnification using the Aperio Scanscope at Sanford Burnham Medical Research Institute (La Jolla, Calif.). Staining was assessed using a semi-quantitative scoring method by an observer masked to the identity of the sections. Significance was determined by Student's t-test.

### Urine Exosome Analysis

Aliquots of 24 h urine collections were thawed and exosomes were purified and concentrated 100-fold by volume exclusion. Exosome protein was measured by Pierce BCA assay and total extra- and intra-exosomal double-strand DNA was quantified by Picogreen fluorescence. Extraexosomal DNA was hydrolyzed by treatment with DNAse I. Intraexosomal mitochondrial DNA (mtDNA) was quantified by real-time quantitative PCR (RT-QPCR) using two primer pairs; one directed to the ND4 region of mtDNA in the major arc, and the other pair directed to the 16S region in the minor arc. Copy numbers of mtDNA are reported in copies per μg of exosomal DNA.

### Intrarenal Gene Expression Analysis

Human renal biopsy specimens were collected in an international multicenter study, the European Renal cDNA Bank—Kröner-Fresenius biopsy bank (ERCB-KFB, see appendix for participating centers) 12. Biopsies were obtained from patients after informed consent and with approval of the local ethics committees. In a hybridization experiment Affymetrix HG-U133A microarrays were initially hybridized with cDNA from cortical tubulo-interstitial specimen 13. Confirmatory real-time RT-PCR analyses were performed on microdissected specimen from clinically indicated biopsies from additional patients with diabetic kidney disease (n=14), minimal change disease (n=6), or pretransplant kidney biopsies from living donors as controls (n=8).

### Statistical Analysis

Distributions of all metabolites were checked, and due to the skewed distributions, natural log transformation was applied to all metabolites, adding 1 where appropriate. To initially compare the screening group with the control group, ANOVA was used, adjusting for age, race, and gender. All 94 metabolites were compared in this initial analysis, and a false discovery rate (FDR) method was used to determine a significance cut point 14. It was determined by this method that 17 metabolites would be carried forward for a validation analysis in the other groups, i.e. the Validation, Type 1 and Type 2 diabetic groups using a p-value significance of 0.0077. Since metabolites were natural log transformed, for ease of interpretation, results are presented as a percentage (95% CI) compared to the healthy control sample for each group, per ml/min/1.73 m2 for eGFR and per doubling for the albumin/creatinine ratio. Percents were obtained using the transformation (eβ−1)*100. SAS V9.2 (Cary, N.C.) was used to perform analysis. Unadjusted geometric means (95% CIs) of the metabolites were also calculated.

In order to determine whether a parsimonious profile could distinguish between the 5 groups, using the 17 metabolites carried forward for validation, we performed a principal components analysis with varimax rotation of the factors. Scree plots were used to determine an adequate number of components, and results of principal component 1 versus principal component 2 were plotted for all groups, i.e. Control, Screening, Validation, Type 1 and Type 2 diabetes. To determine the association of eGFR and urine ACR by group with the validated metabolites, we applied global ANOVAs adjusted for age, race and gender, with separate terms for eGFR or the natural log of urine ACR terms for each group. The screening and validation groups were combined for this analysis.

### Results

### Clinical Characteristics of Screening and Validation Cohorts

The healthy control group consisted of patients primarily drawn from an urban population in the San Diego region. They had a mean age of 37.7 years, 52% were Caucasian and 26% were female. The clinical characteristics of the screening cohort with diabetes and CKD, the validation cohort with diabetes and CKD, and the cohort of patients with either type 1 or type 2 diabetes but without kidney disease are shown in Table 1. The screening cohort consisted exclusively of patients with type 2 diabetes with CKD from an urban San Diego region, who were predominantly male and 42% were Caucasian. The validation group included patients with either type 1 or type 2 diabetes from different regions across the United States and Finland. Although patients in the screening and validation groups were different with respect to ethnicity and geographic location, they were similar with respect to eGFR and overall medical management (Table 1).

Urine Metabolites in Healthy Controls and Diabetic Patients with CKD

94 separate metabolites were measured in the healthy control group (n=23) and the screening cohort (n=24). Using 0.0077 as the FDR significance threshold, 17 metabolites were found to be significantly different in the screening cohort as compared to the healthy controls (Table 2). In the validation cohort of patients with diabetic kidney disease, urine metabolomics were analyzed with the same platform. We observed a high concordance rate of the urine metabolites in the validation group, as urine concentrations of 13 of the 17 metabolites identified in the screening cohort were confirmed to be statistically significantly different from the healthy control sample in the validation cohort (Table 2). In addition, the direction of association was consistent, as all 13 metabolites had lower concentrations in patients with diabetic kidney disease compared to the healthy control sample (Supplemental Table 1).

Differentiating Diabetes Vs Diabetic Kidney Disease with Urine Metabolomics

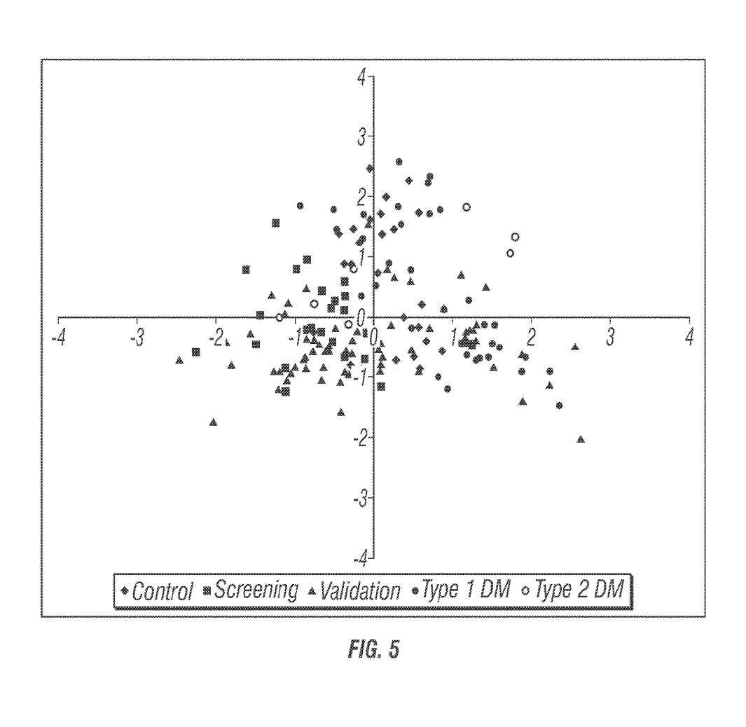

Subjects in the screening and validation groups all had diabetes and reduced eGFR, whereas the control group had neither. Thus, differences between these samples and the healthy controls might be due to the presence of diabetes, the presence of reduced eGFR, or both. To determine if diabetes itself contributed to the observed mean differences in urine metabolites, an independent cohort of patients with type 1 diabetes or type 2 diabetes with intact kidney function (eGFR>75) were evaluated (Supplemental Table 2). Of the 13 metabolites that were consistently different in the screening and validation samples, only one metabolite (3-methyl adipic acid) was found to be consistently and significantly reduced in both the type 1 and type 2 diabetic groups without kidney disease, compared to the healthy controls. An additional two of the 13 metabolites were significantly different in the type 2 diabetic group (3-hydroxyisobutyrate and 2-methyl acetoacetate) compared to the healthy controls. Principal components analysis of the 17 metabolites demonstrated that the first two components separated the groups reasonably well (FIG. 5). The patients with type 2 diabetes alone were isolated on the right horizontal. Interestingly the healthy control group and the type 1 diabetic group co-migrated along the positive vertical axis.

### Biochemical Basis of Metabolite Differences

To determine if any of the metabolites correlated with common markers of kidney disease in diabetes, we evaluated whether the urine concentrations of the metabolites were statistically related to eGFR or albuminuria (ACR) (Supplemental Tables 3a and 4b). Eleven of 13 metabolites showed a significant correlation with eGFR, with 7 demonstrating p values <0.0001. With respect to albuminuria, 3 metabolites were significantly correlated with ACR, although the strength of the associations was weaker overall.

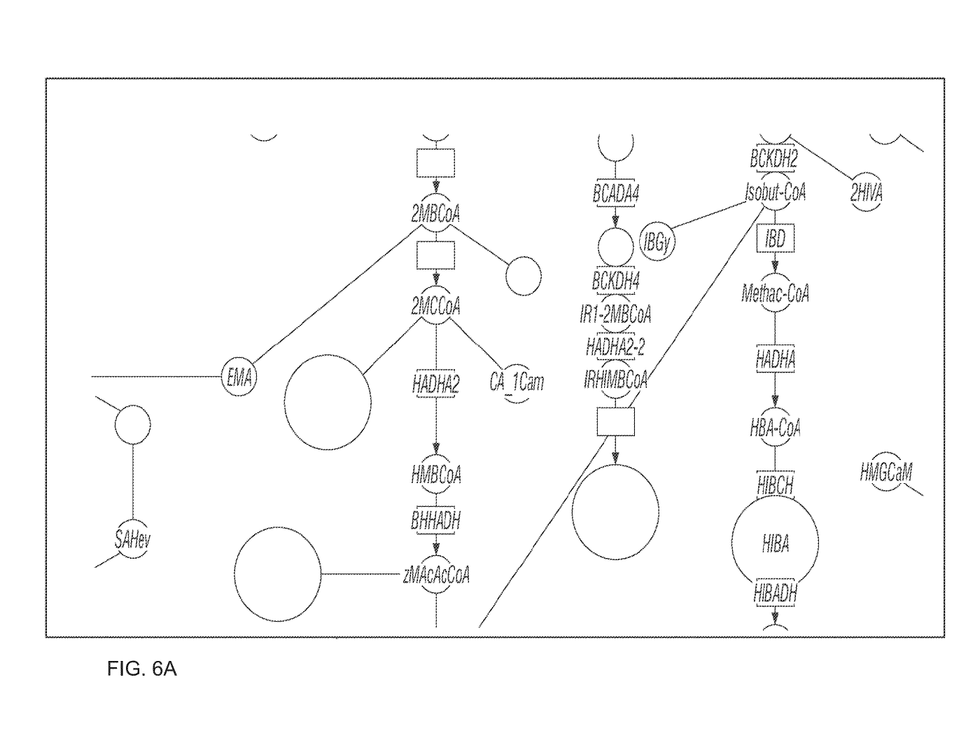

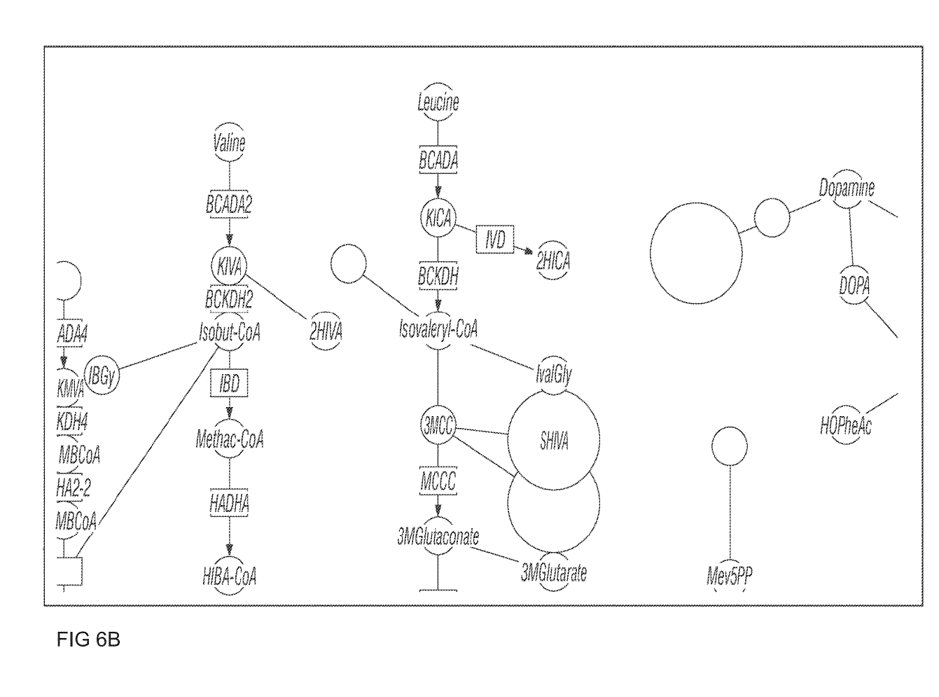

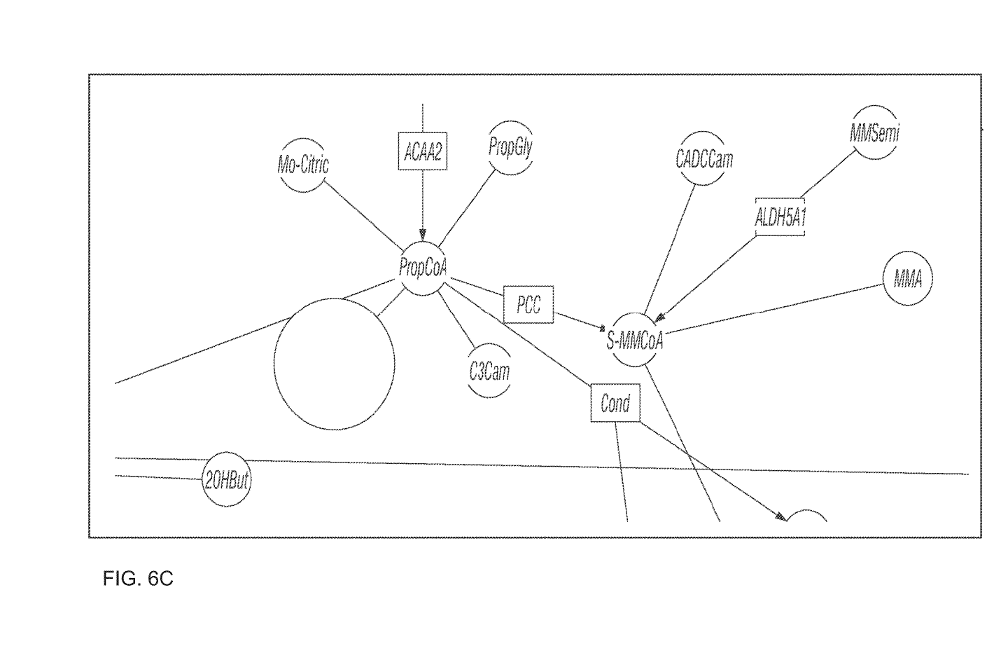

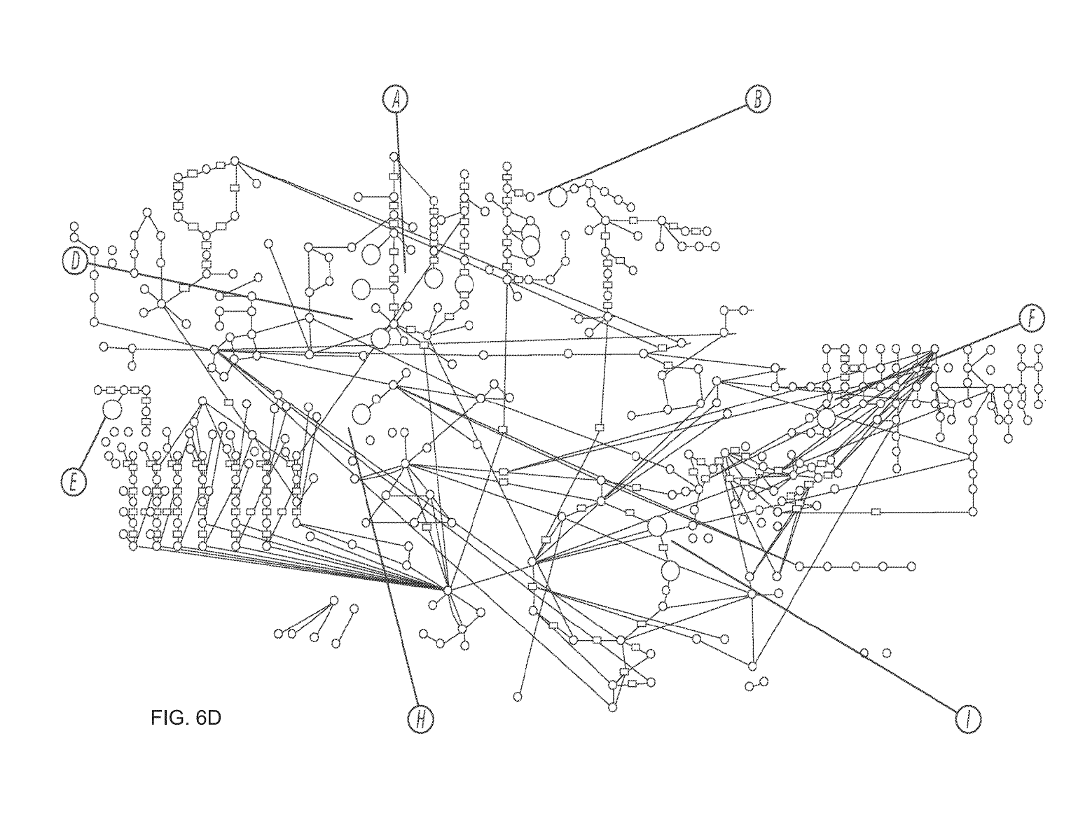

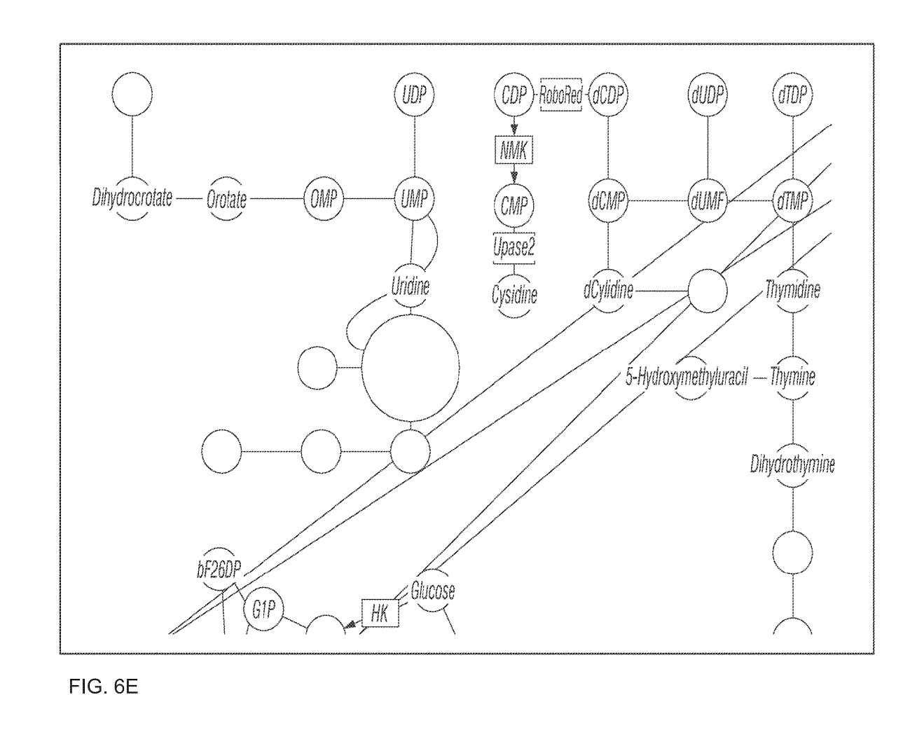

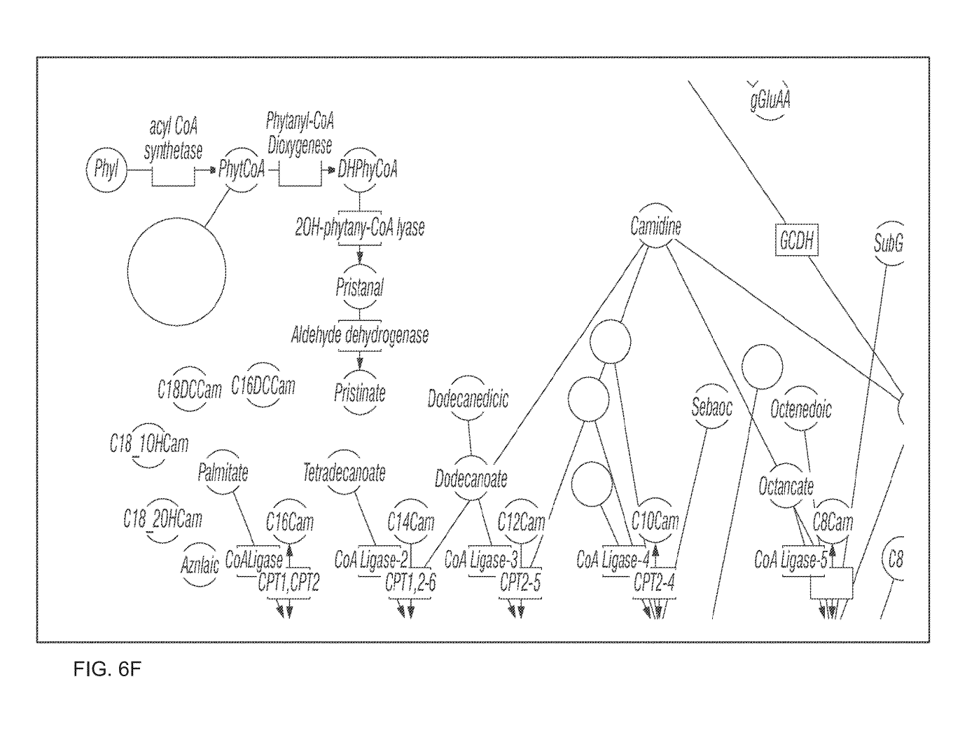

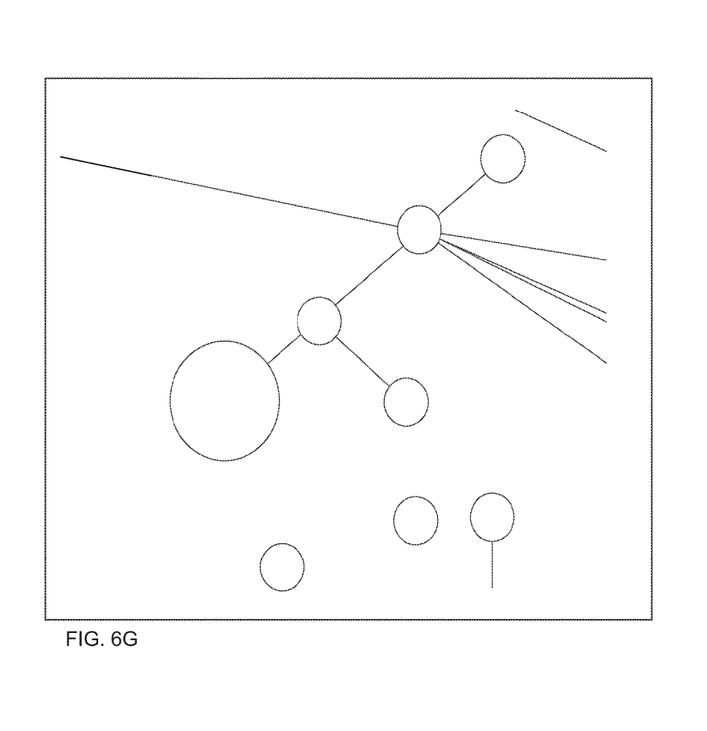

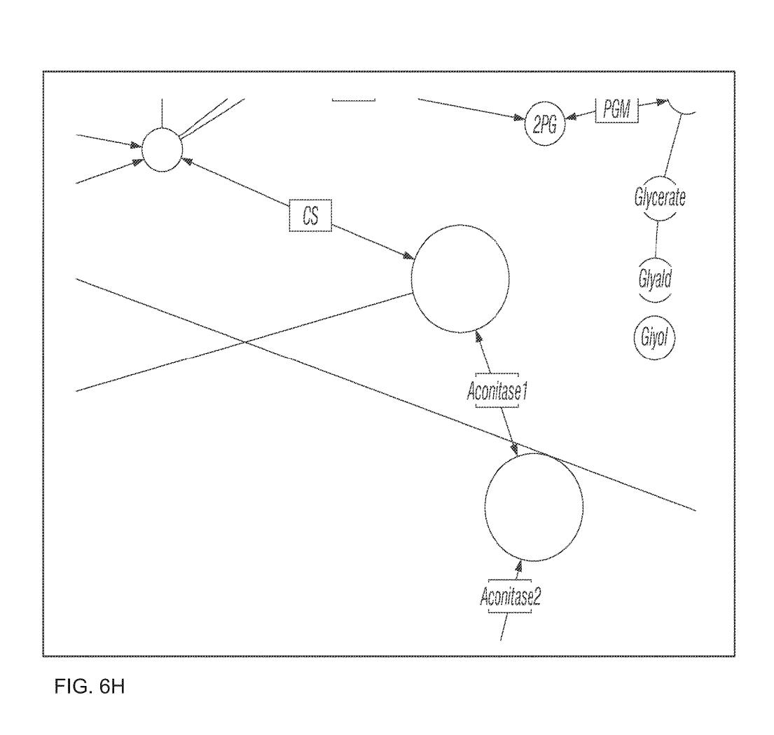

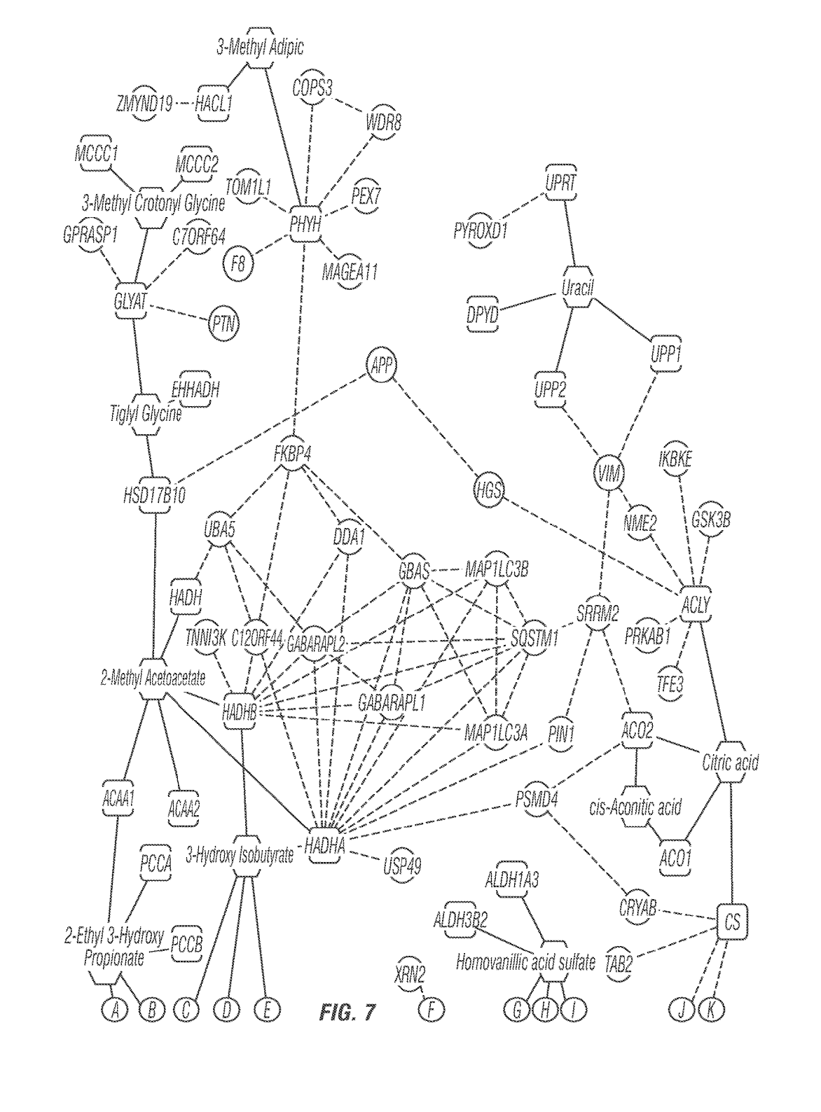

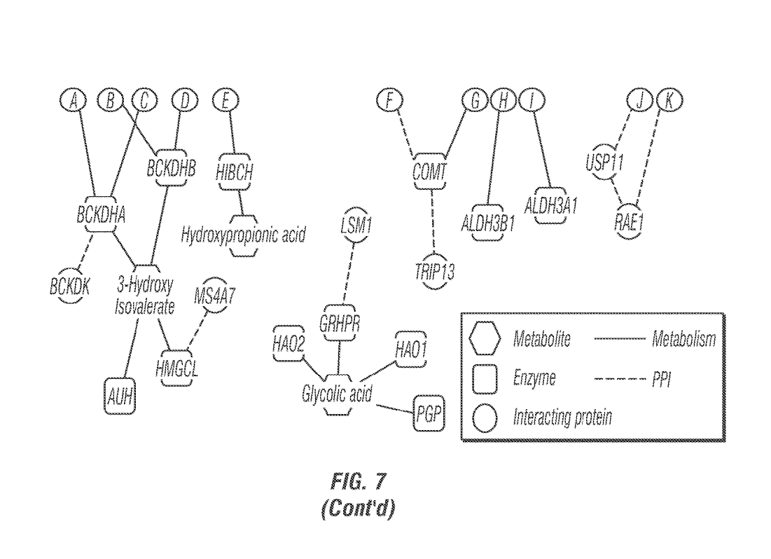

The data separating the diabetic CKD group from the others indicated a series of metabolites which provide a metabolomic signature and may provide biochemical insight. As shown in FIG. 6, metabolites from the Krebs cycle, pyrimidine metabolism, amino acid, propionate, fatty acid and oxalate metabolism were all significantly reduced in the urines of patients with diabetic kidney disease. 11/13 metabolites were connected by network analysis using a protein-protein interaction network linking the enzymes involved in production of the metabolites (FIG. 7). 2/13 of the metabolites (glycolic acid and homovanillic acid) were out of network. A classification table based on organelle localization of the metabolites and the enzymes producing the metabolites identified that 12/13 metabolites that varied significantly in patients with diabetic kidney disease were associated with mitochondria, with 11 exclusively produced in mitochondria (Table 3). As there was a reduction in all of the mitochondrial metabolite markers, the combined biochemical and systems analysis suggests that there may be a generalized reduction in several aspects of mitochondrial function in patients with diabetic kidney disease.

### Validation of Biochemical Pathways

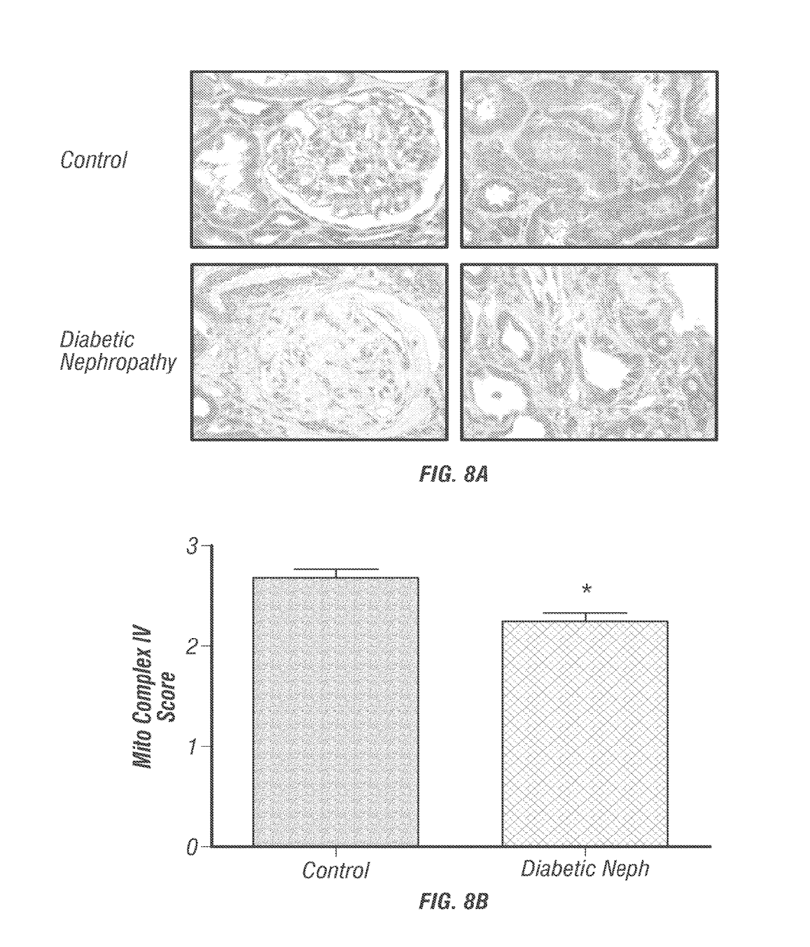

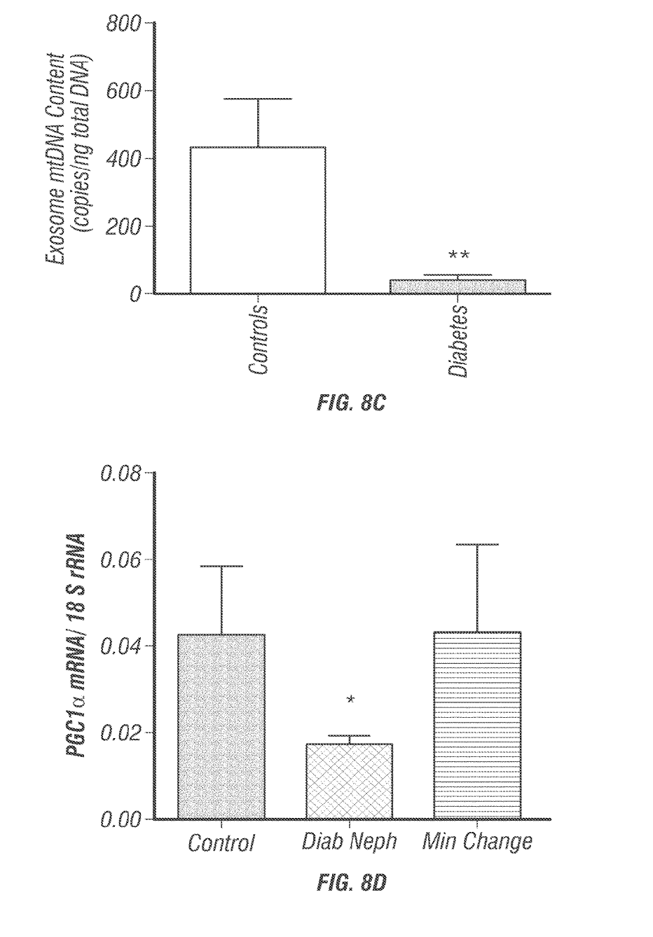

To determine if there was indeed a reduction in mitochondria in patients with diabetic kidney disease, several approaches were pursued. We performed immunostaining with an antibody for cytochrome C oxidase (mitochondrial complex IV) on archived paraffin-embedded tissue sections of kidney biopsy samples from patients with normal kidneys and patients with diabetic nephropathy (n=5 per group). There was a reduction of mitochondrial cytochrome C oxidase (complex IV) in the diabetic kidneys as compared to control kidneys (FIG. 8A, B). Second, measurement of mitochondrial DNA in urine exosomes was performed, as urine exosomes are largely derived from renal epithelial cells 15 and may reflect the intracellular constituents of glomerular and tubular epithelial cells. Consistent with the kidney biopsy findings, we observed a reduction in urine exosomal mtDNA in patients with diabetic kidney disease vs controls (FIG. 8C). The reduction in mitochondrial protein and mtDNA indicate an overall reduction in mitochondrial biogenesis.

To explain a pathway by which mitochondrial proteins may be reduced in diabetic kidney disease, we examined gene expression of PGClalpha, a key regulator of mitochondrial biogenesis16 17. In independent kidney biopsy samples from a European consortium, quantitative RT-PCR for PGClalpha was performed on microdissected cortical tubulo-interstitial samples from patients with diabetic kidney disease, minimal change disease (a non-progressive proteinuric disease), and pre-transplant biopsies as controls (FIG. 8D). The PGClalpha mRNA expression was reduced in samples with diabetic kidney disease (fold-change 0.4, p<0.05), whereas the expression was unchanged in minimal change disease compared with controls.

### Discussion

The results of the present study demonstrate that urine metabolomics can be a clinically useful platform to provide a metabolic signature and provide novel biochemical insights in patients with diabetic kidney disease. We demonstrate that the alteration in the urine metabolome observed in diabetic kidney disease is largely consistent across independent ethnic and geographic groups and is largely driven by diabetic kidney disease, and not diabetes alone. Of 94 urine metabolites that were examined, 13 metabolites were consistently and significantly reduced in urine from patients with diabetic kidney disease compared to healthy controls, and 7 were closely associated with glomerular filtration rate. Using biochemical and systems biology tools, we further demonstrate that diabetic kidney disease is characterized by suppressed mitochondrial function. Independent studies with exosomal analysis and immunohistochemistry validated the hypothesis that there is a reduction in mitochondria in patients with diabetic kidney disease.

A recent study from our collaborators (FinnDiane18) studied the association of urine metabolites with progression to albuminuria over a mean follow up of 5.5 years. Using LC/MS, they identified hippuric acid to be decreased in the group with progressive disease, and S-(3-oxododecanoyl) cysteamine to be increased with progression, and also found that the concentration of acylcarnitines were increased, but did not identify the esters. In our study, we found 4-OH hippurate to be reduced in patients with diabetic kidney disease, similar to the finding for the related hippurate in the FinnDiane study 18; both compounds are glycine esters of a single exogenous metabolite or its hydroxylation derivative.

In our study, 11/13 metabolites that were found to be significantly different in patients with diabetes and CKD were found to be linked by network analysis. The majority of these metabolites are known to be produced in mitochondria or are largely regulated by mitochondrial function. The reduction of mitochondrial function suggested by the metabolomic signature was confirmed with urine exosomal analysis of mtDNA and immunohistochemistry demonstrating reduced mitochondrial content. The basis for reduced mitochondrial content and function will be difficult to address with clinical samples; however in recent work from animal studies our group demonstrated that reduced renal mitochondrial content may be due to signaling pathways affected by hyperglycemia (see companion manuscript). A potential mechanism to explain reduced mitochondrial content in the diabetic kidney is reduction of the co-activator, PGClalpha. In an independent series of samples, PGClalpha mRNA levels were reduced in human diabetic kidneys. As PGClalpha is the major co-activator to regulate mitochondrial biogenesis 16, a reduction in PGClalpha would be expected to lead to reduced mitochondrial biogenesis. Of note, PGClalpha gene expression has been found to be reduced in muscle tissue of patients with type 2 diabetes and may be due to epigenetic alterations of the PGClalpha promoter 17.

The change in urine metabolites may be due to reduction in glomerular filtration itself or may precede and potentially contribute to renal functional decline. As several of the metabolites were not associated with eGFR and ACR there may be an independent regulatory pattern that contribute to the alteration in the urine metabolites. Future longitudinal studies will help to determine if the urine metabolomic signature provides additive predictive value for kidney functional decline and associated co-morbid outcomes.

In conclusion, urine metabolomics provides a novel, non-invasive method to identify biomarkers and biochemical insights that are associated with kidney function and are highly consistent across patient populations with diabetes and kidney disease. The distinct metabolomic signature indicates that mitochondrial function is reduced in patients with diabetic kidney disease relative to healthy controls, and independent studies confirmed this hypothesis. Ultimately, these findings may identify new therapeutic targets for diabetic kidney disease and may serve as additional biomarkers of kidney function, independent of albuminuria.

### Tables

TABLE 1
Baseline characteristics by group

| | Screening | Validation | Type 1 DM | Type 2 DM |
|---|---|---|---|---|
| | DM + CKD | DM + CKD | w/o CKD | w/o CKD |
| Characteristic | n = 24 | n = 82 | n = 27 | n = 25 |
| Age, years | 64.1 ± 7.8 | 60.4 ± 11.2 | 44.4 ± 9.5 | 57.5 ± 6.5 |
| Race | | | | |
| White | 10 (42%) | 59 (72%) | 27 (100%) | 25 (100%) |
| Non-White | 14 (58%) | 23 (28%) | 0 (0%) | 0 (0%) |
| Female Gender | 3 (13%) | 37 (45%) | 12 (44%) | 9 (36%) |
| BMI, kg/m² | 34.2 ± 6.2 | 30.8 ± 7.2 | 25.1 ± 3.2 | 24.2 ± 2.5 |
| Ever Smoking | 14 (58%) | 38 (46%) | 15 (56%) | 18 (72%) |
| Systolic BP, mmHg | 131.8 ± 20.5 | 133.4 ± 14.9 | 138.1 ± 18.2 | 137.3 ± 17.5 |
| Diastolic BP, mmHg | 70.9 ± 10.6 | 74.0 ± 8.8 | 77.6 ± 8.0 | 82.9 ± 9.3 |
| Type 2 DM Duration, | 16.0 (10.0, 21.0) | 13.0 (8.0, 22.0) | — | 11.0 (6.0, 14.0) |
| years* | — | 31.0 (25.0, 38.0) | 31.0 (25.0, 38.0) | — |
| Type 1 DM Duration, | 7.2 ± 1.1 | 7.7 ± 1.4 | 6.2 ± 3.2 | 8.1 ± 1.2 |
| years* | 2.2 ± 0.6 | 1.9 ± 0.9 | 0.7 ± 0.1 | 0.9 ± 0.1 |
| HbA1c, % | 0.81 (0.09, 1.31) | 0.18 (0.05, 0.80) | 0.06 (0.02, 0.95) | 0.08 (0.05, 0.14) |
| Serum Creatinine, mg/dL | 35.5 ± 10.9 | 43.8 ± 11.7 | 112.9 ± 21.9 | 88.9 ± 12.6 |
| Albumin/Creatinine | | | | |
| Ratio* | | | | |
| eGFR, ml/min/1.73 m² | | | | |

*Median (Quartile 1, Quartile 3)

TABLE 2
Comparison of Metabolites in Validation and Screening groups versus healthy control group*

| | Screening | | Validation | |
|---|---|---|---|---|
| | vs. | | vs. | |
| | Control | | Control | |
| Metabolite | Percent (95% CI) | p-value** | Percent (95% CI) | p-value ** |
| 3-Methyl Adipic Acid | −85.82 (−67.25, −93.86) | 2.71700E−05 | −86.81 (−91.18, −80.27) | 1.05344E-16 |
| 2-Methyl Acetoacetate | −75.26 (−36.51, −90.36) | 4.64133E−03 | −85.78 (−91.43, −76.4) | 1.35454E-11 |
| 3-MethylCrotonyl Glycine | −66.72 (−40.8, −81.29) | 2.71700E−05 | −70.88 (−79.91, −57.79) | 2.05079E-09 |
| 3-Hydroxy Propionate | −59.87 (−37.91, −74.07) | 1.27514E−04 | −58.62 (−70.49, −41.98) | 1.15520E-06 |
| 3-Hydroxy Isovalerate | −77.98 (−63.8, −86.6) | 2.46631E−07 | −65.13 (−76.89, −47.38) | 1.75027E-06 |
| 2-Ethyl 3-OH Propionate | −51.03 (−32.76, −64.34) | 4.60544E−05 | −43.84 (−56.02, −28.29) | 8.94167E-06 |
| Glycolic Acid | −67.6 (−53.88, −77.25) | 9.29224E−08 | −48.06 (−61.33, −30.25) | 2.62639E-05 |
| Tiglylglycine | −44.07 (−24.67, −58.47) | 3.03410E−04 | −49.35 (−63.26, −30.19) | 5.69420E-05 |
| Homovanillic Acid | −51.89 (−20.33, −70.95) | 5.49637E−03 | −51.31 (−65.35, −31.58) | 5.87278E-05 |
| Citric Acid | −72.55 (−58.05, −82.03) | 2.40408E−07 | −64.97 (−78.79, −42.13) | 7.09965E-05 |
| 3-Hydroxy Isobutyrate | −61.61 (−39.62, −75.59) | 1.10357E−04 | −55.09 (−72.42, −26.87) | 1.53707E-03 |
| Aconitic Acid | −46.81 (−28.52, −60.41) | 9.59154E−05 | −46.62 (−64.9, −18.81) | 3.72990E-03 |
| Uracil | −67.2 (−38.75, −82.44) | 8.28500E−04 | −42.76 (−62.33, −13.01) | 9.48992E-03 |
| N-Acetyl Asparate | −74.55 (−60.38, −83.65) | 1.78885E−07 | 63.00 (−13.76, 208.07) | 0.1310 |
| Succinic Acid | −63.74 (−27.43, −81.89) | 5.16738E−03 | −18.05 (−45.70, 23.69) | 0.3397 |
| 4-Hydroxy Hippurate | −5.88 (−1.97, −9.63 | 4.44022E−03 | −2.32 (−7.66, 3.32) | 0.4083 |
| 2-Hydroxy Butyrate | −35.34 (−11.47, −52.78) | 7.67354E−03 | 8.98 (−17.69, 44.30) | 0.5447 |

*Adjusted for age, race and gender, and metabolites are ln-transformed.
**P-values are for percents, which are a comparison validation or screening groups to the untreated group; p-values in bold designate significant metabolites by false discovery method

TABLE 3
Subcellular Localization of Enzymes Producing the Metabolites
Associated with Diabetic Kidney Disease.

| Metabolite | | | | |
|---|---|---|---|---|
| Associated with | | Function in | | |
| Diabetic | | Intermediary | Enzyme(s) Producing | Subcellular |
| Kidney Disease | HMDB ID | Metabolism | the Metabolite | Location |
| 3-Hydroxy | HMDB00754 | Leucine | 3-Methyl Glutaconyl | Mitochondria |
| Isovalerate | | metabolite | CoA Hydratase* | |
| (3-OH 3-Methyl | | | | |
| Butyric Acid) | | | | |
| Glycolic Acid | HMDB00115 | Glycine | NADPH-Glyoxylate | Peroxisomes |
| | | (peroxisomes) | Reductase | & |
| | | and 4OH- | | Mitochondria |
| | | Proline | | |
| | | (mitochondria) | | |
| | | metabolite | | |
| Citric Acid | HMDB00094 | Krebs cycle | Citrate Synthase | Mitochondria |
| | | and | | |
| | | lipid synthesis | | |
| 2-Ethyl 3-OH | HMDB00396 | Isoleucine | From R-pathway of | Mitochondria |
| Propionate | | metabolite | isoleucine metabolism | |
| (2-Ethyl | | | (when 2MBDH is | |
| Hydracrylic | | | deficient) | |
| Acid) | | | | |
| Uracil (Undine) | HMDB00300 | Pyrimidine | CoQ10: Dihdyroorotate | Mitochondria |
| | | synthesis | Dehydrogenase, | |
| | | | Uridine | |
| | | | Monophosphate | |
| | | | Synthetase (UMPS) | |
| 3-Hydroxy | HMDB00023 | Valine | 3HIBA CoA | Mitochondria |
| Isobutyrate | | metabolite | Hydratase | |
| Aconitic Acid | HMDB00072 | Krebs cycle | Aconitase | Mitochondria |
| 3-Methyl Adipic | HMDB00555 | Indicates | From decreased Alpha | Peroxisome |
| Acid | | incomplete | Oxidation | |
| | | branched chain | | |
| | | fatty acid | | |
| | | oxidation | | |
| Tiglylglycine | HMDB00959 | Isoleucine | FAD+ 2- | Mitochondria |
| | | metabolite | Methylbutyryl-CoA | |
| | | | Dehydrogenase | |
| | | | (2MBD) | |
| 3-Methyl- | HMDB00459 | Leucine | FAD+ Isovaleryl-CoA | Mitochondria |
| Crotonyl | | metabolite | Dehydrogenase | |
| Glycine | | | | |
| 2-Methyl | HMDB03771 | Isoleucine | NAD+ 2-Methyl-3- | Mitochondria |
| Acetoacetate | | metabolite | Hydroxy Butyryl CoA | |
| | | | Dehydrogenase | |
| Homovanillic | HMDB00118 | Dopamine | Catechol-O-Methyl | Cytosol & |
| Acid | | metabolite | Transferase (COMT) | Mitochondria |
| | | | & Monoamine | |
| | | | Oxidase (MAO) | |
| 3-Hydroxy | HMDB00700 | Isoleucine, | 2-Methylacetoacetyl | Mitochondria |
| Propionate | | Valine, | CoA Thiolase (Ile); | |
| (Hydracrylic | | Threonine, and | NAD+- | |
| acid) | | Methionine | Methylmalonate | |
| | | metabolite | Semialdehyde | |
| | | | Dehydrogenase (Val); | |
| | | | NAD+ 2-Ketobutyrate | |
| | | | Dehydrogenase (Thr & | |
| | | | Met) | |

2MBD: 2-Methylbutyryl-CoA dehydrogenase in Isoleucine metabolism
*Via alternative substrate utilization: 3-Methylbutyryl-CoA vs 3-methylglutaconyl-CoA
^{#}Via alternative substate utilization: 3-Hydroxy-3-methylbutyryl CoA vs 3-hydroxy-3-methylglutaryl-CoA
^{&}As an alternative substrate: 2-Ethyl-3-hydroxypropionyl-CoA vs 2-methyl-3-hydroxybutyryl-CoA

SUPPLEMENTARY TABLE 1
Geometric Mean Metabolite Levels by Group*

| | Control | Screening | Validation |
|---|---|---|---|
| | Geometric Mean | Geometric Mean | Geometric Mean |
| | (95% CI) | (95% CI) | (95% CI) |
| Metabolite | n = 23 | n = 24 | n = 82 |
| 3-Hydroxy Isovalerate | 37.47 (28.93, 48.54) | 11.39 (8.84, 14.68) | 15.85 (13.82, 18.17) |
| 4-Hydroxy Hippurate | 1.06 (1.02, 1.10) | 1.02 (0.98, 1.06) | 1.06 (1.04, 1.08) |
| N-Acetyl Asparate | 6.61 (4.57, 9.58) | 2.25 (1.57, 3.24) | 8.20 (6.74, 9.98) |
| Aconitic Acid | 78.02 (60.94, 99.89) | 54.08 (42.46, 68.88) | 52.40 (45.98, 59.73) |
| Citric Acid | 523.69 (388.37, 706.15) | 201.26 (150.2, 269.68) | 256.22 (218.70, 300.17) |
| 2-Ethyl 3-Hydroxy Propionate | 1.86 (1.58, 2.19) | 1.41 (1.20, 1.65) | 1.38 (1.27, 1.51) |
| Glycolic Acid | 84.39 (69.26, 102.83) | 35.36 (29.14, 42.91) | 46.82 (42.17, 51.98) |
| Homovanillic Acid | 15.77 (12.32, 20.18) | 8.00 (6.28, 10.19) | 10.01 (8.78, 11.41) |
| 3-Hydroxy Isobutyrate | 46.91 (34.87, 63.11) | 27.24 (20.37, 36.41) | 29.56 (25.27, 34.59) |
| 2-Methyl Acetoacetate | 7.77 (5.54, 10.89) | 3.74 (2.68, 5.20) | 1.46 (1.22, 1.75) |
| 3-Methyl Adipic Acid | 8.10 (6.31, 10.4) | 1.88 (1.47, 2.40) | 1.43 (1.26, 1.64) |
| 3-Methyl Crotonyl Glycine | 4.02 (3.04, 5.31) | 1.52 (1.16, 2.00) | 1.36 (1.17, 1.58) |
| 2-Hydroxy Butyrate | 4.23 (3.54, 5.05) | 3.52 (2.95, 4.19) | 4.75 (4.32, 5.22) |
| 3-Hydroxy Propionate | 3.57 (2.79, 4.56) | 1.62 (1.27, 2.05) | 1.71 (1.50, 1.94) |
| Succinic Acid | 28.8 (21.42, 38.71) | 16.78 (12.56, 22.42) | 30.76 (26.30, 35.98) |
| Tiglylglycine | 2.52 (2.00, 3.17) | 1.62 (1.29, 2.03) | 1.48 (1.31, 1.67) |
| Uracil | 10.57 (7.99, 14.00) | 3.05 (2.32, 4.02) | 7.00 (6.03, 8.12) |

*Unadjusted geometric means

SUPPLEMENTAL TABLE 2
Comparison of 13 validated metabolites in Type 1 and
Type 2 diabetic groups versus healthy controls group*

| | Type 1 DM | | Type 2 DM | |
|---|---|---|---|---|
| | w/o CKD | | w/o CKD | |
| | vs. | | vs. | |
| | Control | | Control | |
| Metabolite | Percent (95% CI) | p-value** | Percent (95% CI) | p-value** |
| 3-Hydroxy Isovalerate | 5.41 (−34.38, 69.32) | 0.8239 | −26.78 (−53.39, 15.04) | 0.1713 |
| Aconitic Acid | 5.24 (−24.78, 47.23) | 0.7609 | −6.24 (−25.26, 17.61) | 0.5693 |
| Citric Acid | 21.19 (−12.46, 67.79) | 0.2402 | −15.84 (−38.53, 15.23) | 0.2746 |
| 2-Ethyl 3-OH Propionate | 18.42 (−11.76, 58.91) | 0.2531 | −17.35 (−38.29, 10.71) | 0.1957 |
| Glycolic Acid | 26.68 (−10.94, 80.18) | 0.1831 | −0.9 (−25.09, 31.12) | 0.9486 |
| Homovanillic Acid | −13.53 (−44.75, 35.33) | 0.5166 | 11.76 (−12.65, 43.01) | 0.3679 |
| 3-Hydroxy Isobutyrate | 56.32 (4.91, 132.92) | 0.0290 | −90.27 (−95.91, −76.86) | <.0001 |
| 2-Methyl Acetoacetate | −3.5 (−58.91, 126.66) | 0.9334 | −79.53 (−89.69, −59.33) | <.0001 |
| 3-Methyl Adipic Acid | −61.04 (−78.14, −30.59) | 0.0020 | −55.52 (−75.24, −20.09) | 0.0079 |
| 3-Methyl Crotonyl Glycine | −7.07 (−52.88, 83.29) | 0.8289 | 5.64 (−27.14, 53.16) | 0.7674 |
| 3-Hydroxy Propionate | −35.01 (−57.56, −0.49) | 0.0475 | 8.03 (−39.12, 91.68) | 0.7873 |
| Tiglylglycine | −29.07 (−54.18, 9.81) | 0.1204 | −23.01 (−49.99, 18.53) | 0.2284 |
| Uracil | −10.73 (−41.34, 35.86) | 0.5890 | 72.44 (9.58, 171.38) | 0.0197 |

*Adjusted for age, race and gender, and metabolites are ln-transformed
**P-values are for percents, which are a comparison of validation/screening, type 1 or type 2 groups to the untreated group; p-values in bold designate significant metabolites by false discovery method

SUPPLEMENTAL TABLE 3a
Association of eGFR with Metabolites
in patients with Diabetes and CKD*

| | Type 1 and 2 | |
|---|---|---|
| | DM + CKD | |
| | % change in eGFR | |
| | with change in | |
| | metabolite | |
| Metabolite | Percent (95% CI) | p |
| 3-Hydroxy Isovalerate | 2.65 (2.03, 3.27) | 1.1721E−14 |
| Glycolic Acid | 1.94 (1.47, 2.42) | 1.2290E−13 |
| Citric Acid | 2.80 (2.02, 3.59) | 4.1044E−11 |
| 2-Ethyl 3 OH Propionate | 1.09 (0.68, 1.50) | 4.8216E−07 |
| Uracil | 2.04 (1.20, 2.89) | 3.3314E−06 |
| 3-Hydroxy Isobutyrate | 1.92 (1.12, 2.72) | 4.1345E−06 |
| Aconitic Acid | 1.64 (0.94, 2.35) | 7.9426E−06 |
| 3-Methyl Adipic Acid | 0.93 (0.35, 1.52) | 0.0019 |
| Tiglylglycine | 0.95 (0.27, 1.63) | 0.0063 |
| Homovanillic Acid | 0.94 (0.23, 1.66) | 0.0101 |
| 3-Methyl Crotonyl Glycine | 1.00 (0.21, 1.80) | 0.0137 |
| 3-Hydroxy Propionate | 0.58 (−0.16, 1.33) | 0.1261 |
| 2-Methyl Acetoacetate | 0.39 (−0.67, 1.45) | 0.4746 |

*Metabolites are ln-transformed and eGFR untransformed in ml/min/1.73 m²; models adjusted for age, race and gender

SUPPLEMENTAL TABLE 3b
Association of Albumin-Creatinine Ratio (ACR) with
Metabolites in patients with Diabetes and CKD*

| | Type 1 and 2 | |
|---|---|---|
| | DM + CKD | |
| | Percent change in | |
| | ACR with change in | |
| Metabolite | metabolite (95% CI) | p |
| 3-Hydroxy Isovalerate | 2.45 (−2.26, 7.4) | 0.3111 |
| Aconitic Acid | 0.37 (−4.29, 5.26) | 0.8769 |
| Citric Acid | 0.45 (−4.98, 6.18) | 0.8743 |
| 2-Eth 3 OH Propionate | 3.09 (0.22, 6.05) | 0.0346 |
| Glycolic Acid | −0.35 (−3.73, 3.16) | 0.8436 |
| 3-Methyl Adipic Acid | 4.36 (0.52, 8.34) | 0.0261 |
| 3-Hydroxy Isobutyrate | 3.68 (−1.76, 9.42) | 0.1871 |
| 3-Hydroxy Propionate | 1.05 (−3.54, 5.87) | 0.6566 |
| Tiglylglycine | 2.08 (−2.21, 6.55) | 0.3450 |
| 3-Methyl Crotonyl | 4.62 (−0.59, 10.09) | 0.0824 |
| Glycine | −4.51 (−9.63, 0.9) | 0.1002 |
| Uracil | 10.50 (3.00, 18.55) | 0.0057 |
| 2-Methyl Acetoacetate | 1.37 (−3.18, 6.13) | 0.5600 |
| Homovanillic Acid | | |

*Ln-transformed albumin/creatinine ratio and metabolites; results are presented per doubling of albumin creatinine ratio, i.e. each doubling albumin/creatinine ratio corresponds to the given percent higher or lower metabolite; models adjusted for age, race and gender.

### REFERENCES FOR THIS EXAMPLE

1. Rosolowsky E T, Skupien J, Smiles A M, et al. Risk for ESRD in type 1 diabetes remains high despite renoprotection. J Am Soc Nephrol 2011; 22:545-53.
2. Groop P H, Thomas M C, Moran J L, et al. The presence and severity of chronic kidney disease predicts all-cause mortality in type 1 diabetes. Diabetes 2009; 58:1651-8.
3. Susztak K, Bottinger E, Novetsky A, et al. Molecular profiling of diabetic mouse kidney reveals novel genes linked to glomerular disease. Diabetes 2004; 53:784-94.
4. Ewens K G, George R A, Sharma K, Ziyadeh F N, Spielman R S. Assessment of 115 candidate genes for diabetic nephropathy by transmission/disequilibrium test. Diabetes 2005; 54:3305-18.
5. Sharma K, Lee S, Han S, et al. Two-dimensional fluorescence difference gel electrophoresis analysis of the urine proteome in human diabetic nephropathy. Proteomics 2005; 5:2648-55.
6. Wang T J, Larson M G, Vasan R S, et al. Metabolite profiles and the risk of developing diabetes. Nature medicine 2011; 17:448-53.
7. Sweetman L, Nyhan W L. Detailed comparison of the urinary excretion of purines in a patient with the Lesch-Nyhan syndrome and a control subject. Biochem Med 1971; 4:121-34.
8. Nyhan W L, James J A, Teberg A J, Sweetman L, Nelson L G. A new disorder of purine metabolism with behavioral manifestations. J Pediatr 1969; 74:20-7.
9. Aramaki S, Lehotay D, Nyhan W L, MacLeod P M, Sweetman L. Methylcitrate in maternal urine during a pregnancy with a fetus affected with propionic acidaemia. J Inherit Metab Dis 1989; 12:86-8.
10. Sharma K, Ix J H, Mathew A V, et al. Pirfenidone for diabetic nephropathy. J Am Soc Nephrol 2011; 22:1144-51.
11. Hoffmann G, Aramaki S, Blum-Hoffmann E, Nyhan W L, Sweetman L. Quantitative analysis for organic acids in biological samples: batch isolation followed by gas chromatographic-mass spectrometric analysis. Clin Chem 1989; 35:587-95.
12. Cohen C D, Frach K, Schlondorff D, Kretzler M. Quantitative gene expression analysis in renal biopsies: a novel protocol for a high-throughput multicenter application. Kidney Int 2002; 61:133-40.
13. Schmid H, Boucherot A, Yasuda Y, et al. Modular activation of nuclear factor-kappaB transcriptional programs in human diabetic nephropathy. Diabetes 2006; 55:2993-3003.
14. Benjamini Y, Hochberg Y. Controlling the false discovery rate: A practical and powerful approach to multiple testing. J Royal Statistical Society, Series B 1995; 57:289-300.
15. Zhou H, Cheruvanky A, Hu X, et al. Urinary exosomal transcription factors, a new class of biomarkers for renal disease. Kidney Int 2008; 74:613-21.
16. Spiegelman B M. Transcriptional control of mitochondrial energy metabolism through the PGC1 coactivators. Novartis Found Symp 2007; 287:60-3; discussion 3-9.
17. Barres R, Osler M E, Yan J, et al. Non-CpG methylation of the PGC-lalpha promoter through DNMT3B controls mitochondrial density. Cell Metab 2009; 10:189-98.
18. van der Kloet F M, Tempels F W, Ismail N, et al. Discovery of early-stage biomarkers for diabetic kidney disease using ms-based metabolomics (FinnDiane study). Metabolomics 2012; 8:109-19.

### Example 11

### Representative Embodiments

This example describes exemplary embodiments of the invention:

1. A method of identifying the presence or level of kidney disease in a subject, comprising
a. determining the level of at least one metabolite selected from the group consisting of 3-methyl adipic acid, 2-methyl acetoacetate, 3-methyl crotonyl glycine, 3-hydroxy propionate, 2-ethyl 3-OH propionate, and tiglylglycine in a sample obtained from the subject;
b. comparing the level of the at least one metabolite with a reference level of the at least one metabolite, wherein
   i. the reference level has been determined from at least one sample collected from the same subject at a different time period; or
   ii. the reference level has been determined from a sample or samples collected from one or more other subjects; and
c. identifying the presence or level of kidney disease in the subject where the at least one metabolite level in the subject is decreased when compared to the reference level of the at least one metabolite.
   2. The method of embodiment 1, further comprising
d. determining the level of at least one additional metabolite selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject,
e. comparing the level of the at least one additional metabolite with a reference level of the at least one additional metabolite, wherein
   i. the reference level has been determined from at least one sample collected from the same subject at a different time period; or
   ii. the reference level has been determined from a sample or samples collected from one or more other subjects; and
f. identifying the presence or level of kidney disease in the subject where the at least one metabolite and the at least one additional metabolite levels in the subject are decreased when compared to the reference levels of the at least one metabolite and the at least one additional metabolite.
   3. The method of embodiment 1, further comprising
d. determining the level of at least two additional metabolites selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least two additional metabolites with reference levels of the at least two additional metabolites, wherein
   i. the reference levels have been determined from at least one sample collected from the same subject at a different time period; or
   ii. the reference levels have been determined from a sample or samples collected from one or more other subjects; and
f. identifying the presence or level of kidney disease in the subject where the at least one metabolite and the at least two additional metabolite levels in the subject are decreased when compared to the reference levels of the at least one metabolite and the at least two additional metabolites.
   4. The method of embodiment 1, further comprising
d. determining the level of at least three additional metabolites selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least three additional metabolites with reference levels of the at least two additional metabolites, wherein
   i. the reference levels have been determined from at least one sample collected from the same subject at a different time period; or
   ii. the reference levels have been determined from a sample or samples collected from one or more other subjects; and
f. identifying the presence or level of kidney disease in the subject where the at least one metabolite and the at least three additional metabolite levels in the subject are decreased when compared to the reference levels of the at least one metabolite and the at least three additional metabolites.
   5. The method of embodiment 1, further comprising
d. determining the level of at least four additional metabolites selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least four additional metabolites with reference levels of the at least two additional metabolites, wherein
   i. the reference levels have been determined from at least one sample collected from the same subject at a different time period; or
   ii. the reference levels have been determined from a sample or samples collected from one or more other subjects; and
f. identifying the presence or level of kidney disease in the subject where the at least one metabolite and the at least four additional metabolite levels in the subject are decreased when compared to the reference levels of the at least one metabolite and the at least four additional metabolites.
   6. The method of embodiment 1, further comprising
d. determining the level of at least five additional metabolites selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least five additional metabolites with reference levels of the at least two additional metabolites, wherein
   i. the reference levels have been determined from at least one sample collected from the same subject at a different time period; or
   ii. the reference levels have been determined from a sample or samples collected from one or more other subjects; and
f. identifying the presence or level of kidney disease in the subject where the at least one metabolite and the at least five additional metabolite levels in the subject are decreased when compared to the reference levels of the at least one metabolite and the at least five additional metabolites.
   7. The method of embodiment 1, further comprising
d. determining the level of at least six additional metabolites selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least six additional metabolites with reference levels of the at least two additional metabolites, wherein
   i. the reference levels have been determined from at least one sample collected from the same subject at a different time period; or
   ii. the reference levels have been determined from a sample or samples collected from one or more other subjects; and
f. identifying the presence or level of kidney disease in the subject where the at least one metabolite and the at least six additional metabolite levels in the subject are decreased when compared to the reference levels of the at least one metabolite and the at least six additional metabolites.
   8. The method of embodiment 1, further comprising
d. determining the level of at least seven additional metabolites selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least seven additional metabolites with reference levels of the at least two additional metabolites, wherein
   i. the reference levels have been determined from at least one sample collected from the same subject at a different time period; or
   ii. the reference levels have been determined from a sample or samples collected from one or more other subjects; and
f. identifying the presence or level of kidney disease in the subject where the at least one metabolite and the at least seven additional metabolite levels in the subject are decreased when compared to the reference levels of the at least one metabolite and the at least seven additional metabolites.
   9. The method of embodiment 1, further comprising
d. determining the level of at least eight additional metabolites selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least eight additional metabolites with reference levels of the at least two additional metabolites, wherein
   i. the reference levels have been determined from at least one sample collected from the same subject at a different time period; or
   ii. the reference levels have been determined from a sample or samples collected from one or more other subjects; and
f. identifying the presence or level of kidney disease in the subject where the at least one metabolite and the at least eight additional metabolite levels in the subject are decreased when compared to the reference levels of the at least one metabolite and the at least eight additional metabolites.
   10. The method of embodiment 1, further comprising
d. determining the level of at least nine additional metabolites selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least nine additional metabolites with reference levels of the at least two additional metabolites, wherein
   i. the reference levels have been determined from at least one sample collected from the same subject at a different time period; or
   ii. the reference levels have been determined from a sample or samples collected from one or more other subjects; and
f. identifying the presence or level of kidney disease in the subject where the at least one metabolite and the at least nine additional metabolite levels in the subject are decreased when compared to the reference levels of the at least one metabolite and the at least nine additional metabolites.
   11. The method of embodiment 1, further comprising
d. determining the level of at least ten additional metabolites selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least ten additional metabolites with reference levels of the at least two additional metabolites, wherein
   i. the reference levels have been determined from at least one sample collected from the same subject at a different time period; or
   ii. the reference levels have been determined from a sample or samples collected from one or more other subjects; and
f. identifying the presence or level of kidney disease in the subject where the at least one metabolite and the at least ten additional metabolite levels in the subject are decreased when compared to the reference levels of the at least one metabolite and the at least ten additional metabolites.
   12. The method of embodiment 1, further comprising
d. determining the level of at least eleven additional metabolites selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least eleven additional metabolites with reference levels of the at least two additional metabolites, wherein
   i. the reference levels have been determined from at least one sample collected from the same subject at a different time period; or
   ii. the reference levels have been determined from a sample or samples collected from one or more other subjects; and
f. identifying the presence or level of kidney disease in the subject where the at least one metabolite and the at least eleven additional metabolite levels in the subject are decreased when compared to the reference levels of the at least one metabolite and the at least eleven additional metabolites.
   13. The method of embodiment 1, further comprising
d. determining the level of at least twelve additional metabolites selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least twelve additional metabolites with reference levels of the at least two additional metabolites, wherein
   i. the reference levels have been determined from at least one sample collected from the same subject at a different time period; or
   ii. the reference levels have been determined from a sample or samples collected from one or more other subjects; and
f. identifying the presence or level of kidney disease in the subject where the at least one metabolite and the at least twelve additional metabolite levels in the subject are decreased when compared to the reference levels of the at least one metabolite and the at least twelve additional metabolites.
   14. The method of any of embodiments 1 to 13, wherein the subject has diabetes.
   15. The method of any of embodiments 1 to 13, wherein the subject has diabetic kidney disease.
   16. The method of any of embodiments 1 to 15, wherein the reference level of the metabolite is determined from a sample obtained from a healthy patient.
   17. The method of any of embodiments 1 to 16, wherein the reference level of the metabolite is determined from a sample obtained from the subject at an earlier time.
   18. The method of any of embodiments 1 to 16, wherein the reference level of the metabolite is determined from an analysis of samples obtained from more than one healthy patient.
   19. The method of any of embodiments 1 to 18, wherein the level of the metabolite or acids is decreased at least 1.5 fold compared to the reference level.
   20. The method of any of 1 to 18, wherein the level of the metabolite or acids is decreased at least 2 fold compared to the reference level.
   21. The method of any of 1 to 18, wherein the level of the metabolite or acids is decreased at least 3 fold compared to the reference level.
   22. The method of any of 1 to 18, wherein the level of the metabolite or acids is decreased at least 4 fold compared to the reference level.
   23. The method of any of embodiments 1 to 22, wherein the subject has not been diagnosed with diabetes.
   24. The method of any of embodiments 1 to 23, wherein the subject has kidney disease.
   25. The method of any of embodiments 1 to 24, wherein the level of the metabolite is determined using gas chromatography.
   26. The method of any of embodiments 1 to 25, wherein the level of the metabolite is determined using mass spectrometry.
   27. The method of any of embodiments 1 to 26, wherein the level of the metabolite is determined from a biological sample from the subject.
   28. The method of embodiment 27, wherein the sample contains urine, or a urine fraction, or blood, or a blood fraction.
   29. A method of determining the progression of kidney disease over time in a subject diagnosed with kidney disease, comprising
a. determining the level of at least one organic acid selected from the group consisting of 3-methyl adipic acid, 2-methyl acetoacetate, 3-methyl crotonyl glycine, 3-hydroxy propionate, 2-ethyl 3-OH propionate, and tiglylglycine, in a sample obtained from the subject;
b. comparing the level of the at least one organic acid to the level of the at least one organic acid determined in a sample obtained from the subject at an earlier time point;
c. determining that the kidney disease has progressed in the subject where the at least one organic acid level in the subject is decreased when compared to the level determined in the sample obtained from the subject at the earlier time point.
   30. The method of claim 29, further comprising
d. determining the level of at least one additional organic acid selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least one additional organic acid with a reference level of the at least one additional organic acid, wherein
   i. the reference level has been determined from at least one sample collected from the same subject at a different time period; or
   ii. the reference level has been determined from a sample or samples collected from one or more other subjects; and
f. determining that the kidney disease has progressed in the subject where the at least one organic acid and the at least one additional organic acid levels in the subject are decreased when compared to the level determined in the sample obtained from the subject at the earlier time point.
   31. The method of claim 29, further comprising
d. determining the level of at least two additional organic acids selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least two additional organic acids with reference levels of the at least two additional organic acids, wherein
   i. the reference level has been determined from at least one sample collected from the same subject at a different time period; or
   ii. the reference level has been determined from a sample or samples collected from one or more other subjects; and
f. determining that the kidney disease has progressed in the subject where the at least one organic acid and the at least two additional organic acid levels in the subject are decreased when compared to the levels determined in the sample obtained from the subject at the earlier time point.
   32. The method of embodiment 29, further comprising
d. determining the level of at least three additional metabolites selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least three additional metabolites with reference levels of the at least three additional metabolites, wherein
   i. the reference level has been determined from at least one sample collected from the same subject at a different time period; or
   ii. the reference level has been determined from a sample or samples collected from one or more other subjects; and
f. determining that the kidney disease has progressed in the subject where the at least one metabolite and the at least three additional metabolite levels in the subject are decreased when compared to the levels determined in the sample obtained from the subject at the earlier time point.
   33. The method of embodiment 29, further comprising
d. determining the level of at least four additional metabolites selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least four additional metabolites with reference levels of the at least four additional metabolites, wherein
   i. the reference level has been determined from at least one sample collected from the same subject at a different time period; or
   ii. the reference level has been determined from a sample or samples collected from one or more other subjects; and
f. determining that the kidney disease has progressed in the subject where the at least one metabolite and the at least four additional metabolite levels in the subject are decreased when compared to the levels determined in the sample obtained from the subject at the earlier time point.
   34. The method of embodiment 29, further comprising
d. determining the level of at least five additional metabolites selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least five additional metabolites with reference levels of the at least five additional metabolites, wherein
   i. the reference level has been determined from at least one sample collected from the same subject at a different time period; or
   ii. the reference level has been determined from a sample or samples collected from one or more other subjects; and
f. determining that the kidney disease has progressed in the subject where the at least one metabolite and the at least five additional metabolite levels in the subject are decreased when compared to the levels determined in the sample obtained from the subject at the earlier time point.
   35. The method of embodiment 29, further comprising
d. determining the level of at least six additional metabolites selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least six additional metabolites with reference levels of the at least six additional metabolites, wherein
   i. the reference level has been determined from at least one sample collected from the same subject at a different time period; or
   ii. the reference level has been determined from a sample or samples collected from one or more other subjects; and
f. determining that the kidney disease has progressed in the subject where the at least one metabolite and the at least six additional metabolite levels in the subject are decreased when compared to the levels determined in the sample obtained from the subject at the earlier time point.
   36. The method of embodiment 29, further comprising
d. determining the level of at least seven additional metabolites selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least seven additional metabolites with reference levels of the at least seven additional metabolites, wherein
   i. the reference level has been determined from at least one sample collected from the same subject at a different time period; or
   ii. the reference level has been determined from a sample or samples collected from one or more other subjects; and
f. determining that the kidney disease has progressed in the subject where the at least one metabolite and the at least seven additional metabolite levels in the subject are decreased when compared to the levels determined in the sample obtained from the subject at the earlier time point.
   37. The method of embodiment 29, further comprising
d. determining the level of at least eight additional metabolites selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least eight additional metabolites with reference levels of the at least eight additional metabolites, wherein
   i. the reference level has been determined from at least one sample collected from the same subject at a different time period; or
   ii. the reference level has been determined from a sample or samples collected from one or more other subjects; and
f. determining that the kidney disease has progressed in the subject where the at least one metabolite and the at least eight additional metabolite levels in the subject are decreased when compared to the levels determined in the sample obtained from the subject at the earlier time point.
   38. The method of embodiment 29, further comprising
d. determining the level of at least nine additional metabolites selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least nine additional metabolites with reference levels of the at least nine additional metabolites, wherein
   i. the reference level has been determined from at least one sample collected from the same subject at a different time period; or
   ii. the reference level has been determined from a sample or samples collected from one or more other subjects; and
f. determining that the kidney disease has progressed in the subject where the at least one metabolite and the at least nine additional metabolite levels in the subject are decreased when compared to the levels determined in the sample obtained from the subject at the earlier time point.
   39. The method of embodiment 29, further comprising
d. determining the level of at least ten additional metabolites selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least ten additional metabolites with reference levels of the at least ten additional metabolites, wherein
   i. the reference level has been determined from at least one sample collected from the same subject at a different time period; or
   ii. the reference level has been determined from a sample or samples collected from one or more other subjects; and
f. determining that the kidney disease has progressed in the subject where the at least one metabolite and the at least ten additional metabolite levels in the subject are decreased when compared to the levels determined in the sample obtained from the subject at the earlier time point.
   40. The method of embodiment 29, further comprising
d. determining the level of at least eleven additional metabolites selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least eleven additional metabolites with reference levels of the at least eleven additional metabolites, wherein
   i. the reference level has been determined from at least one sample collected from the same subject at a different time period; or
   ii. the reference level has been determined from a sample or samples collected from one or more other subjects; and
f. determining that the kidney disease has progressed in the subject where the at least one metabolite and the at least eleven additional metabolite levels in the subject are decreased when compared to the levels determined in the sample obtained from the subject at the earlier time point.
   41. The method of embodiment 29, further comprising
d. determining the level of at least twelve additional metabolites selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least twelve additional metabolites with reference levels of the at least twelve additional metabolites, wherein
   i. the reference level has been determined from at least one sample collected from the same subject at a different time period; or
   ii. the reference level has been determined from a sample or samples collected from one or more other subjects; and
f. determining that the kidney disease has progressed in the subject where the at least one metabolite and the at least twelve additional metabolite levels in the subject are decreased when compared to the levels determined in the sample obtained from the subject at the earlier time point.
   42. The method of any of embodiments 29 to 41, wherein the subject has diabetes.
   43. The method of any of embodiments 29 to 41, wherein the subject has diabetic kidney disease.
   44. The method of any of embodiments 29 to 43, wherein the level of the metabolite or acids is decreased at least 1.5 fold compared to the level in the sample obtained from the subject at the earlier time point.
   45. The method of any of embodiments 29 to 43, wherein the level of the metabolite or acids is decreased at least 2 fold compared to the level in the sample obtained from the subject at the earlier time point.
   46. The method of any of embodiments 29 to 45, wherein the subject has not been diagnosed with diabetes.
   47. The method of any of embodiments 29 to 46, wherein the level of the metabolite is determined using gas chromatography.
   48. The method of any of embodiments 29 to 46, wherein the level of the metabolite is determined using mass spectrometry.
   49. The method of any of embodiments 29 to 46, wherein the level of the metabolite is determined from a biological sample from the subject.
   50. The method of embodiment 49, wherein the sample contains urine or a urine fraction, or blood or a blood fraction.
   51. A method comprising:
a. administering a therapeutic to a subject diagnosed with kidney disease;
b. determining the level of at least one metabolite selected from the group consisting of 3-methyl adipic acid, 2-methyl acetoacetate, 3-methyl crotonyl glycine, 3-hydroxy propionate, 2-ethyl 3-OH propionate, and tiglylglycine, in a sample obtained from the subject after administration of the therapeutic;
c. comparing the level of the at least one metabolite to the level of the at least one metabolite determined in a sample obtained from the subject at an earlier time point;
d. administering another dose of the therapeutic to the subject where the level of the at least one metabolite in the sample obtained after administration is not decreased compared to the level of the at least one metabolite in the sample obtained at the earlier time point.
   52. The method of embodiment 51, further comprising
e. determining the level of at least one additional metabolite selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
f. comparing the level of the at least one additional metabolite to the level of the at least one additional metabolite determined in a sample obtained from the subject at an earlier time point;
g. administering another dose of the therapeutic to the subject where the level of the at least one metabolite and the level of the at least one additional organic in the sample obtained after administration is not decreased compared to the level of the at least one metabolite and the level of the at least one additional metabolite in the sample obtained at the earlier time point.
   53. The method of embodiment 51 or 52, wherein another dose of the therapeutic is administered to the subject where the level of the at lease one metabolite or the at least one additional metabolite is not decreased 2 fold.
   54. The method of any of embodiments 51 to 53, wherein the subject has diabetes.
   55. The method of any of embodiments 51 to 53, wherein the subject has diabetic kidney disease.
   56. The method of any of embodiments 51 to 53, wherein the subject has not been diagnosed with diabetes.
   57. The method of any of embodiments 51 to 56, wherein the level of the metabolite is determined from a biological sample from the subject.
   58. The method of embodiment 57, wherein the sample contains urine or a urine fraction, or blood or a blood fraction.
   59. A method for reducing toxicity of a treatment, comprising:
a. determining the pre-treatment level of at least one metabolite selected from the group consisting of 3-methyl adipic acid, 2-methyl acetoacetate, 3-methyl crotonyl glycine, 3-hydroxy propionate, 2-ethyl 3-OH propionate, and tiglylglycine, in a sample obtained from a subject;
b. administering a therapeutic to the subject;
c. determining the post-treatment level of the at least one metabolite, in a sample obtained from the subject after step b; and
d. lowering the subsequent dosage of the therapeutic where the post-treatment level of the at least one metabolite is decreased compared to the pre-treatment level of the at least one metabolite in the sample.
   60. The method of embodiment 59, further comprising determining the pre-treatment and post-treatment levels of at least one additional metabolite selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;

and lowering the subsequent dosage of the therapeutic where the post-treatment levels of the at least one metabolite and the least one additional metabolite are decreased compared to the pre-treatment levels of the at least one metabolite and the at least one additional metabolite in the sample.

61. The method of embodiment 59, further comprising determining the pre-treatment and the post-treatment levels of at least two additional metabolites selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;

and lowering the subsequent dosage of the therapeutic where the post-treatment levels of the at least one metabolite and the least two additional metabolites are decreased compared to the pre-treatment levels of the at least one metabolite and the at least two additional metabolites in the sample.

62. The method of embodiment 59, further comprising determining the pre-treatment and the post-treatment levels of at least three additional metabolites selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;

and lowering the subsequent dosage of the therapeutic where the post-treatment levels of the at least one metabolite and the least three additional metabolites are decreased compared to the pre-treatment levels of the at least one metabolite and the at least three additional metabolites in the sample.

63. The method of embodiment 59, further comprising determining the pre-treatment and the post-treatment levels of at least four additional metabolites selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;

and lowering the subsequent dosage of the therapeutic where the post-treatment levels of the at least one metabolite and the least four additional metabolites are decreased compared to the pre-treatment levels of the at least one metabolite and the at least four additional metabolites in the sample.

64. The method of any of embodiments 59 to 63, wherein the level of the metabolite is determined from a biological sample from the subject.
65. The method of any of embodiments 59 to 63, wherein the sample contains urine or a urine fraction or blood or a blood fraction.
66. The method of any of embodiments 59 to 63, wherein the level of the metabolite is determined using mass spectrometry.
67. A method of identifying the presence or level of diabetes related complications in a subject, comprising
a. determining the level of at least one metabolite selected from the group consisting of 3-methyl adipic acid, 2-methyl acetoacetate, 3-methyl crotonyl glycine, 3-hydroxy propionate, 2-ethyl 3-OH propionate, and tiglylglycine in a sample obtained from the subject;
b. comparing the level of the at least one metabolite with a reference level of the at least one metabolite, wherein
   i. the reference level has been determined from at least one sample collected from the same subject at a different time period; or
   ii. the reference level has been determined from a sample or samples collected from one or more other subjects; and
c. identifying the presence or level of diabetes-related complications in the subject where the at least one metabolite level in the subject is decreased when compared to the reference level of the at least one metabolite.
   68. The method of embodiment 67, further comprising
d. determining the level of at least one additional metabolite selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least one additional metabolite with a reference level of the at least one additional metabolite, wherein
   i. the reference level has been determined from at least one sample collected from the same subject at a different time period; or
   ii. the reference level has been determined from a sample or samples collected from one or more other subjects; and
f. identifying the presence or level of diabetes-related complications in the subject where the level of the at least one metabolite and the level of the at least one additional metabolite in the subject is decreased when compared to the reference levels of the at least one metabolite and the at least one additional metabolite.
   69. The method of embodiments 67 or 68, wherein the diabetes related complication is a microvascular complication.
   70. The method of embodiments 67 or 68, wherein the diabetes related complication is a macrovascular complication.
   71. The method of any of embodiments 67 to 70, wherein the reference level of the metabolite is determined from a sample obtained from a healthy patient.
   72. The method of any of embodiments 67 to 70, wherein the reference level of the metabolite is determined from a sample obtained from the subject at an earlier time.
   73. The method of any of embodiments 67 to 70, wherein the reference level of the metabolite is determined from an analysis of samples obtained from more than one healthy patient.
   74. The method of any of embodiments 67 to 70, wherein the level of the metabolite is determined from a biological sample from the subject.
   75. A method of determining the progression of a diabetes related complication over time in a subject diagnosed with a diabetes related complication, comprising
a. determining the level of at least one metabolite selected from the group consisting of 3-methyl adipic acid, 2-methyl acetoacetate, 3-methyl crotonyl glycine, 3-hydroxy propionate, 2-ethyl 3-OH propionate, and tiglylglycine in a sample obtained from the subject;
b. comparing the level of the at least one metabolite to the level of the at least one metabolite determined in a sample obtained from the subject at an earlier time point;
c. determining that the diabetes related complication has progressed in the subject where the at least one metabolite level in the subject is decreased when compared to the level determined in the sample obtained from the subject at the earlier time point.
   76. The method of embodiment 75, further comprising
d. determining the level of at least one additional metabolite selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least one additional metabolite to the level of the at least one additional metabolite determined in a sample obtained from the subject at an earlier time point;
f. determining that the diabetes related complication has progressed in the subject where the levels of the at least one metabolite and the at least one additional metabolite in the subject are decreased when compared to the levels determined in the sample obtained from the subject at the earlier time point.
   77. A method of identifying the presence or level of diabetes, cardiovascular disease, hypertension, or chronic kidney disease in an obese subject, comprising
a. determining the level of at least one metabolite selected from the group consisting of 3-methyl adipic acid, 2-methyl acetoacetate, 3-methyl crotonyl glycine, 3-hydroxy propionate, 2-ethyl 3-OH propionate, and tiglylglycine in a sample obtained from the subject;
b. comparing the level of the at least one metabolite with a reference level of the at least one metabolite, wherein
   i. the reference level has been determined from at least one sample collected from the same subject at a different time period; or
   ii. the reference level has been determined from a sample or samples collected from one or more other subjects; and
c. identifying the presence or level of diabetes, cardiovascular disease, hypertension, or chronic kidney disease in the subject where the at least one metabolite level in the subject is decreased when compared to the reference level of the at least one metabolite.
   78. The method of embodiment 77, further comprising
d. determining the level of at least one additional metabolite selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least one additional metabolite with a reference level of the at least one metabolite, wherein
   i. the reference level has been determined from at least one sample collected from the same subject at a different time period; or
   ii. the reference level has been determined from a sample or samples collected from one or more other subjects; and
f. identifying the presence or level of diabetes, cardiovascular disease, hypertension, or chronic kidney disease in the subject where the levels of the at least one metabolite and the at least one additional metabolite in the subject is decreased when compared to the reference levels of the at least one metabolite and the at least one additional metabolite.
   79. The method of embodiment 77 or 78, wherein the sample contains urine, a urine fraction, blood, or a blood fraction.
   80. A method of identifying the presence or level of hypertension in a subject, comprising
a. determining the level of at least one metabolite selected from the group consisting of 3-methyl adipic acid, 2-methyl acetoacetate, 3-methyl crotonyl glycine, 3-hydroxy propionate, 2-ethyl 3-OH propionate, and tiglylglycine in a sample obtained from the subject;
b. comparing the level of the at least one metabolite with a reference level of the at least one metabolite, wherein
   i. the reference level has been determined from at least one sample collected from the same subject at a different time period; or
   ii. the reference level has been determined from a sample or samples collected from one or more other subjects; and
c. identifying the presence or level of hypertension in the subject where the at least one metabolite level in the subject is decreased when compared to the reference level of the at least one metabolite.
   81. The method of embodiment 80, further comprising
d. determining the level of at least one additional metabolite selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least one additional metabolite with a reference level of the at least one additional metabolite, wherein
   i. the reference level has been determined from at least one sample collected from the same subject at a different time period; or
   ii. the reference level has been determined from a sample or samples collected from one or more other subjects; and
f. identifying the presence or level of hypertension in the subject where the levels of the at least one metabolite and the at least one additional metabolite in the subject is decreased when compared to the reference levels of the at least one metabolite and the at least one additional metabolite.
   82. The method of embodiment 80 or 81, wherein the sample contains urine, a urine fraction, blood, or a blood fraction.
   83. A method of identifying the presence or level of liver disease in a subject having obesity, diabetes, or chronic kidney disease, comprising
a. determining the level of at least one metabolite selected from the group consisting of 3-methyl adipic acid, 2-methyl acetoacetate, 3-methyl crotonyl glycine, 3-hydroxy propionate, 2-ethyl 3-OH propionate, and tiglylglycine in a sample obtained from the subject;
b. comparing the level of the at least one metabolite with a reference level of the at least one metabolite, wherein
   i. the reference level has been determined from at least one sample collected from the same subject at a different time period; or
   ii. the reference level has been determined from a sample or samples collected from one or more other subjects; and
c. identifying the presence or level of liver disease in the subject where the at least one metabolite level in the subject is decreased when compared to the reference level of the at least one metabolite.
   84. The method of embodiment 83, further comprising
d. determining the level of at least one additional metabolite selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least one additional metabolite with a reference level of the at least one metabolite, wherein
   i. the reference level has been determined from at least one sample collected from the same subject at a different time period; or
   ii. the reference level has been determined from a sample or samples collected from one or more other subjects; and
f. identifying the presence or level of liver disease in the subject where the levels of the at least one metabolite and the at least one additional metabolite in the subject are decreased when compared to the reference levels of the at least one metabolite and the at least one additional metabolite.
   85. The method of embodiments 83 or 84, wherein the sample contains urine, a urine fraction, blood, or a blood fraction.
   86. A method of identifying the presence or level of joint involvement in a subject having obesity, diabetes, or chronic kidney disease, comprising
a. determining the level of at least one metabolite selected from the group consisting of 3-methyl adipic acid, 2-methyl acetoacetate, 3-methyl crotonyl glycine, 3-hydroxy propionate, 2-ethyl 3-OH propionate, and tiglylglycine in a sample obtained from the subject;
b. comparing the level of the at least one metabolite with a reference level of the at least one metabolite, wherein
   i. the reference level has been determined from at least one sample collected from the same subject at a different time period; or
   ii. the reference level has been determined from a sample or samples collected from one or more other subjects; and
c. identifying the presence or level of joint involvement in the subject where the at least one metabolite level in the subject is decreased when compared to the reference level of the at least one metabolite.
   87. The method of embodiment 86, further comprising
d. determining the level of at least one additional metabolite selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least one additional metabolite with a reference level of the at least one metabolite, wherein
   i. the reference level has been determined from at least one sample collected from the same subject at a different time period; or
   ii. the reference level has been determined from a sample or samples collected from one or more other subjects; and
f. identifying the presence or level of joint involvement in the subject where the levels of the at least one metabolite and the at least one additional metabolite in the subject are decreased when compared to the reference levels of the at least one metabolite and the at least one additional metabolite.
   88. The method of embodiments 87 or 88, wherein the sample contains urine, a urine fraction, blood, or a blood fraction.
   89. A method of identifying the presence or level of sleep apnea, restrictive lung disease or obstructive lung disease in a subject having diabetes, obesity, or chronic kidney disease, comprising
a. determining the level of at least one metabolite selected from the group consisting of 3-methyl adipic acid, 2-methyl acetoacetate, 3-methyl crotonyl glycine, 3-hydroxy propionate, 2-ethyl 3-OH propionate, and tiglylglycine in a sample obtained from the subject;
b. comparing the level of the at least one metabolite with a reference level of the at least one metabolite, wherein
   i. the reference level has been determined from at least one sample collected from the same subject at a different time period; or
   ii. the reference level has been determined from a sample or samples collected from one or more other subjects; and
c. identifying the presence or level of sleep apnea, restrictive lung disease or obstructive lung disease in the subject where the at least one metabolite level in the subject is decreased when compared to the reference level of the at least one metabolite.
   90. The method of embodiment 89, further comprising
d. determining the level of at least one additional metabolite selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
e. comparing the level of the at least one additional metabolite with a reference level of the at least one metabolite, wherein
   i. the reference level has been determined from at least one sample collected from the same subject at a different time period; or
   ii. the reference level has been determined from a sample or samples collected from one or more other subjects; and
f. identifying the presence or level of sleep apnea, restrictive lung disease or obstructive lung disease in the subject where the levels of the at least one metabolite and the at least one additional metabolite in the subject are decreased when compared to the reference levels of the at least one metabolite and the at least one additional metabolite.
   91. The method of embodiments 89 or 90, wherein the sample contains urine, a urine fraction, blood, or a blood fraction.
   92. A method of identifying the presence or level of kidney disease in a subject, comprising
a. determining the level of a panel of metabolites comprising glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in a sample obtained from the subject;
b. comparing the level of the panel of metabolites with a reference level of the panel of metabolites, wherein
   i. the reference level has been determined from at least one sample collected from the same subject at a different time period; or
   ii. the reference level has been determined from a sample or samples collected from one or more other subjects; and
c. identifying the presence or level of kidney disease in the subject wherein the level of at least one, two, three, four, five, six, seven, eight, nine, ten, eleven, twelve, or thirteen metabolites in the subject is decreased when compared to the reference levels of the at least one, two, three, four, five, six, seven, eight, nine, ten, eleven, twelve, or thirteen metabolites, wherein the thirteen metabolites are selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine.
   93. The method of embodiment 92 wherein the levels of thirteen metabolites, wherein the metabolites are selected from the group consisting of glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine are decreased when compared to the reference levels of the thirteen metabolites.
   94. A method comprising
a. obtaining a sample from a subject;
b. detecting the amount of a panel of metabolites comprising glycolic acid, 3-hydroxy isobutyrate, 3-hydroxy isovalerate, aconitic acid, homovanillic acid, citric acid, uracil, 3-methyl adipic acid, 2-methyl acetoacetate, 3-methyl crotonyl glycine, 3-hydroxy propionate, 2-ethyl 3-OH propionate, and tiglylglycine in the sample; and
c. comparing said amount to the amount of the panel of metabolites in a sample obtained from the subject at an earlier time point.
   95. The method of embodiment 94, further comprising providing an outcome based on the comparison of step (c).
   96. The method of any of embodiments 94 or 95, wherein the subject is human.
   97. The method of any of embodiments 94 to 96, wherein the subject has diabetes.
   98. The method of any of embodiments 94 to 96, wherein the subject has diabetic kidney disease.
   99. The method of any of embodiments 94 to 96, wherein the subject is obese.

### Embodiment A

A method comprising:

(1) (a) administering a therapeutic to a subject diagnosed with kidney disease;

b. determining the level of at least one organic acid selected from the group consisting of glycolic acid, 3-OH isobutyric acid, 3-OH isovaleric acid, aconitic acid, homovanillic acid, citric acid, uracil, fumaric acid, oleic acid and azelaic acid, in a sample obtained from the subject; and

c. determining whether the dosage of the therapeutic subsequently administered to the subject is adjusted based on the level of the at least one organic acid; or

(2) (a) determining the level of at least one organic acid selected from the group consisting of glycolic acid, 3-OH isobutyric acid, 3-OH isovaleric acid, aconitic acid, homovanillic acid, citric acid, uracil, fumaric acid, oleic acid and azelaic acid, in a sample obtained from a subject diagnosed with kidney disease, wherein the subject has been administered a therapeutic; and

b. maintaining a subsequent dosage of the therapeutic or adjusting a subsequent dosage of the therapeutic administered to the subject based on the level of the at least one organic acid in the sample.

### Embodiment B

The method of EMBODIMENT A:

(a) comprising determining the level of at least two organic acids selected from the group consisting of glycolic acid, 3-OH isobutyric acid, 3-OH isovaleric acid, aconitic acid, homovanillic acid, citric acid, uracil, fumaric acid, oleic acid and azelaic acid, are determined, and maintaining a subsequent dosage of the therapeutic or adjusting a subsequent dosage of the therapeutic administered to the subject based on the levels of at least two organic acids in the sample;

(b) further comprising determining the level of 5-oxoproline in a sample obtained from the subject, and maintaining a subsequent dosage of the therapeutic or adjusting a subsequent dosage of the therapeutic administered to the subject based on the levels of 5-oxoproline in the sample;

(c) further comprising determining the level of citrate in a sample obtained from the subject, and maintaining a subsequent dosage of the therapeutic or adjusting a subsequent dosage of the therapeutic administered to the subject based on the levels of citrate in the sample;

(d) wherein the subject has diabetes or diabetic kidney disease, or the subject has not been diagnosed with diabetes;

(e) the level of the organic acid, the 5-oxoproline, or the citrate is determined using gas chromatography or mass spectrometry, or the level of the organic acid is determined from a biological sample from the subject; or

(f) the sample contains urine or a urine fraction, or blood or a blood fraction.

### Embodiment C

A method for reducing toxicity of a treatment, comprising:

(a) (i) determining the pre-treatment level of at least one organic acid selected from the group consisting of glycolic acid, 3-OH isobutyric acid, 3-OH isovaleric acid, aconitic acid, homovanillic acid, citric acid, uracil, fumaric acid, oleic acid and azelaic acid, in a sample obtained from the subject;

(ii) administering a therapeutic to the subject;

(iii) determining the post-treatment level of the at least one organic acid selected from the group consisting of glycolic acid, 3-OH isobutyric acid, 3-OH isovaleric acid, aconitic acid, homovanillic acid, citric acid, uracil, fumaric acid, oleic acid and azelaic acid, in the subject after step b; and

(iv) lowering the subsequent dosage of the therapeutic where the post-treatment level of the at least one organic acid is decreased compared to the pre-treatment level of the at least one organic acid in the sample;

(b) the method of (a), comprising determining the levels of at least two organic acids selected from the group consisting of glycolic acid, 3-OH isobutyric acid, 3-OH isovaleric acid, aconitic acid, homovanillic acid, citric acid, uracil, fumaric acid, oleic acid and azelaic acid, and lowering the subsequent dosage of the therapeutic where the post-treatment levels of the at least two organic acids are decreased compared to the pre-treatment levels of the at least two organic acids in the sample;

(c) the method of (a) or (b), further comprising determining the pre-treatment level of 5-oxoproline, in a sample obtained from the subject, determining the post-treatment level of 5-oxoproline, in the subject, and lowering the subsequent dosage of the therapeutic where the post-treatment level of 5-oxoproline is increased compared to the pre-treatment level of 5-oxoproline in the sample;

(d) the method of any of (a) to (c), further comprising determining the pre-treatment level of citrate, in the subject, determining the post-treatment level of citrate, in a sample obtained from the subject, and lowering the subsequent dosage of the therapeutic where the post-treatment level of citrate is decreased compared to the pre-treatment level of citrate in the sample;

(e) the method of any of (a) to (d), wherein the level of the organic acid is determined from a biological sample from the subject;

(f) the method of any of (a) to (e), wherein the sample contains urine or a urine fraction or blood or a blood fraction; or

(g) the method of any of (a) to (f), wherein the level of the organic acid is determined using mass spectrometry.

### Embodiment D

A method of identifying the presence or level of diabetes related complications in a subject, comprising:

(1) (a) determining the level of at least one metabolite selected from the group consisting of lactic acid, glycolic acid, fumaric acid, malic acid, adipic acid, 2-OH-glutaric acid, aconitic acid, homovanillic acid, stearic acid, 3-OH-isobutyric acid, palmitic acid, and citrate in a sample obtained from the subject;

b. comparing the level of the at least one metabolite with a reference level of the at least one metabolite, wherein
i. the reference level has been determined from at least one sample collected from the same subject at a different time period; or
ii. the reference level has been determined from a sample or samples collected from one or more other subjects; and

c. identifying the presence or level of diabetes-related complications in the subject where the at least one metabolite level in the subject is decreased when compared to the reference level of the at least one metabolite;

(2) the method of (1), wherein the level of at least two metabolites are determined, compared to at least two reference metabolites, and the presence or level of diabetes related complications in the subject is identified where the at least metabolite levels in the subject are decreased when compared to the at least two reference metabolite levels;

(3) the method (1) or (2), wherein the diabetes related complication is a microvascular complication;

(4) the method any of (1) to (3), wherein the diabetes related complication is a macrovascular complication;

(5) the method any of (1) to (4), wherein the reference level of the metabolite is determined from a sample obtained from a healthy patient;

(6) the method any of (1) to (5), wherein the reference level of the metabolite is determined from a sample obtained from the subject at an earlier time;

(7) the method any of (1) to (6), wherein the reference level of the metabolite is determined from an analysis of samples obtained from more than one healthy patient;

(8) the method any of (1) to (7), wherein the level of the metabolite is determined from a biological sample from the subject; or

(9) the method any of (1) to (8), wherein the sample contains urine, or a urine fraction, or blood, or a blood fraction.

### Embodiment E

A method of determining the progression of a diabetes related complication over time in a subject diagnosed with a diabetes related complication, comprising

(a) (i) determining the level at least one metabolite selected from the group consisting of lactic acid, glycolic acid, fumaric acid, malic acid, adipic acid, 2-OH-glutaric acid, aconitic acid, homovanillic acid, stearic acid, 3-OH-isobutyric acid, palmitic acid, and citrate in a sample obtained from the subject;

(ii). comparing the level of the at least one metabolite to the level of the at least one metabolite determined in a sample obtained from the subject at an earlier time point; and

(iii) determining that the diabetes related complication has progressed in the subject where the at least one metabolite level in the subject is decreased when compared to the level determined in the sample obtained from the subject at the earlier time point;

(b) the method of (a), wherein the level of the metabolite is determined from a biological sample from the subject;

(c) the method of (a) or (b), wherein the sample contains urine or a urine fraction; or

(d) the method of any of (a) to (c), wherein the sample contains blood or a blood fraction.

### Embodiment F

A method comprising:

a. administering a therapeutic to a subject diagnosed with a diabetes related complication;

b. determining the level of at least one metabolite selected from the group consisting of lactic acid, glycolic acid, fumaric acid, malic acid, adipic acid, 2-OH-glutaric acid, aconitic acid, homovanillic acid, stearic acid, 3-OH-isobutyric acid, palmitic acid, and citrate in a sample obtained from the subject; and

c. determining whether the dosage of the therapeutic subsequently administered to the subject is adjusted based on the level of the at least one metabolite.

### Embodiment G

A method of identifying the presence or level of diabetes, cardiovascular disease, hypertension, or chronic kidney disease in an obese subject, comprising

(1) (a) determining the level of at least one metabolite selected from the group consisting of lactic acid, glycolic acid, fumaric acid, malic acid, adipic acid, 2-OH-glutaric acid, aconitic acid, homovanillic acid, stearic acid, 3-OH-isobutyric acid, palmitic acid, and citrate in a sample obtained from the subject;

(b) comparing the level of the at least one metabolite with a reference level of the at least one metabolite, wherein
i. the reference level has been determined from at least one sample collected from the same subject at a different time period; or
ii. the reference level has been determined from a sample or samples collected from one or more other subjects; and

(c) identifying the presence or level of diabetes-related complications in the subject where the at least one metabolite level in the subject is decreased when compared to the reference level of the at least one metabolite; or

(2) the method of (1), wherein the sample contains urine, a urine fraction, blood, or a blood fraction;

### Embodiment H

A method of identifying the presence or level of diabetes, cardiovascular disease, hypertension, or chronic kidney disease in an obese subject, comprising

(1) (a) determining the level of at least one metabolite selected from the group consisting of lactic acid, glycolic acid, fumaric acid, malic acid, adipic acid, 2-OH-glutaric acid, aconitic acid, homovanillic acid, stearic acid, 3-OH-isobutyric acid, palmitic acid, and citrate in a sample obtained from the subject;

(b) comparing the level of the at least one metabolite with a reference level of the at least one metabolite, wherein
i. the reference level has been determined from at least one sample collected from the same subject at a different time period; or
ii. the reference level has been determined from a sample or samples collected from one or more other subjects; and

(c) identifying the presence or level of diabetes, cardiovascular disease, hypertension, or chronic kidney disease in the subject where the at least one metabolite level in the subject is decreased when compared to the reference level of the at least one metabolite;

(2) the method of (1), wherein the sample contains urine, a urine fraction, blood, or a blood fraction.

### Embodiment I

A method of identifying the presence or level of hypertension in a subject, comprising

(1) (a) determining the level of at least one metabolite selected from the group consisting of lactic acid, glycolic acid, fumaric acid, malic acid, adipic acid, 2-OH-glutaric acid, aconitic acid, homovanillic acid, stearic acid, 3-OH-isobutyric acid, palmitic acid, and citric acid in a sample obtained from the subject;

(b) comparing the level of the at least one metabolite with a reference level of the at least one metabolite, wherein
i. the reference level has been determined from at least one sample collected from the same subject at a different time period; or
ii. the reference level has been determined from a sample or samples collected from one or more other subjects; and

(c) identifying the presence or level of hypertension in the subject where the at least one metabolite level in the subject is decreased when compared to the reference level of the at least one metabolite.

(2) the method of (1), wherein the sample contains urine, a urine fraction, blood, or a blood fraction.

### Embodiment J

A method of identifying the presence or level of liver disease in a subject having obesity, diabetes, or chronic kidney disease, comprising

(1) (a) determining the level of at least one metabolite selected from the group consisting of lactic acid, glycolic acid, fumaric acid, malic acid, adipic acid, 2-OH-glutaric acid, aconitic acid, homovanillic acid, stearic acid, 3-OH-isobutyric acid, palmitic acid, and citric acid in a sample obtained from the subject;

(b) comparing the level of the at least one metabolite with a reference level of the at least one metabolite, wherein
i. the reference level has been determined from at least one sample collected from the same subject at a different time period; or
ii. the reference level has been determined from a sample or samples collected from one or more other subjects; and

(c) identifying the presence or level of liver disease in the subject where the at least one metabolite level in the subject is decreased when compared to the reference level of the at least one metabolite.

(2) the method of (1), wherein the sample contains urine, a urine fraction, blood, or a blood fraction.

### Embodiment K

A method of identifying the presence or level of joint involvement in a subject having obesity, diabetes, or chronic kidney disease, comprising

(1) (a) determining the level of at least one metabolite selected from the group consisting of lactic acid, glycolic acid, fumaric acid, malic acid, adipic acid, 2-OH-glutaric acid, aconitic acid, homovanillic acid, stearic acid, 3-OH-isobutyric acid, palmitic acid, citric acid, and 5-oxoproline in a sample obtained from the subject;

(b) comparing the level of the at least one metabolite with a reference level of the at least one metabolite, wherein
i. the reference level has been determined from at least one sample collected from the same subject at a different time period; or
ii. the reference level has been determined from a sample or samples collected from one or more other subjects; and

(c) identifying the presence or level of joint involvement in the subject where the at least one metabolite level in the subject is decreased when compared to the reference level of the at least one metabolite.

(2) the method of (1), wherein the sample contains urine, a urine fraction, blood, or a blood fraction.

### Embodiment L

A method of identifying the presence or level of sleep apnea, restrictive lung disease or obstructive lung disease in a subject having diabetes, obesity, or chronic kidney disease, comprising

(1) (a) determining the level of at least one metabolite selected from the group consisting of lactic acid, glycolic acid, fumaric acid, malic acid, adipic acid, 2-OH-glutaric acid, aconitic acid, homovanillic acid, stearic acid, 3-OH-isobutyric acid, palmitic acid, citric acid, and 5-oxoproline in a sample obtained from the subject;

(b) comparing the level of the at least one metabolite with a reference level of the at least one metabolite, wherein
i. the reference level has been determined from at least one sample collected from the same subject at a different time period; or
ii. the reference level has been determined from a sample or samples collected from one or more other subjects; and

(c) identifying the presence or level of sleep apnea, restrictive lung disease or obstructive lung disease in the subject where the at least one metabolite level in the subject is decreased when compared to the reference level of the at least one metabolite.

(2) the method of (1), wherein the sample contains urine, a urine fraction, blood, or a blood fraction.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

The description presented herein is merely exemplary in nature and is in no way intended to limit the scope of the invention, its application, or uses, which may vary. The technology is described with relation to the non-limiting definitions and terminology included herein. These definitions and terminology are not designed to function as a limitation on the scope or practice of the technology, but are presented for illustrative and descriptive purposes only. Various terms used throughout the specification and claims are defined as set forth herein as it may be helpful to an understanding of the technology discussed herein.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim that follow.

What is claimed is:

1. A method of identifying the presence or level of kidney disease in a subject, comprising
a. determining the level of at least one organic acid selected from the group consisting of glycolic acid, 3-OH isobutyric acid, 3-OH isovaleric acid, aconitic acid, homovanillic acid, citric acid, uracil, fumaric acid, oleic acid and azelaic acid in a sample obtained from the subject;
b. comparing the level of the at least one organic acid with a reference level of the at least one organic acid, wherein
i. the reference level has been determined from at least one sample collected from the same subject at a different time period; or
ii. the reference level has been determined from a sample or samples collected from one or more other subjects; and
c. identifying the presence or level of kidney disease in the subject where the at least one organic acid level in the subject is decreased when compared to the reference level of the at least one organic acid.

2. The method of claim 1, wherein the level of:
(a) at least two organic acids selected from the group consisting of glycolic acid, 3-OH isobutyric acid, 3-OH isovaleric acid, aconitic acid, homovanillic acid, citric acid, uracil, fumaric acid, oleic acid, and azelaic acid, are determined, compared to at least two reference organic acids, and the presence or level of kidney disease in the subject is identified where the at least two organic acid levels in the subject are decreased when compared to the at least two reference organic acid levels;
(b) at least three organic acids selected from the group consisting of glycolic acid, 3-OH isobutyric acid, 3-OH isovaleric acid, aconitic acid, homovanillic acid, citric acid, uracil, fumaric acid, oleic acid, and azelaic acid, are determined, compared to at least three reference organic acids, and the presence or level of kidney disease in the subject is identified where the at least three organic acid levels in the subject are decreased when compared to the at least three reference organic acid levels;
(c) least four organic acids selected from the group consisting of glycolic acid, 3-OH isobutyric acid, 3-OH isovaleric acid, aconitic acid, homovanillic acid, citric acid, uracil, fumaric acid, oleic acid, and azelaic acid, are determined, compared to at least four reference organic acids, and the presence or level of kidney disease in the subject is identified where the at least four organic acid levels in the subject are decreased when compared to the at least four reference organic acid levels;
(d) at least five organic acids selected from the group consisting of glycolic acid, 3-OH isobutyric acid, 3-OH isovaleric acid, aconitic acid, homovanillic acid, citric acid, uracil, fumaric acid, oleic acid, and azelaic acid, are determined, compared to at least five reference organic acids, and the presence or level of kidney disease in the subject is identified where the at least five organic acid levels in the subject are decreased when compared to the at least five reference organic acid levels;
(e) at least six organic acids selected from the group consisting of glycolic acid, 3-OH isobutyric acid, 3-OH isovaleric acid, aconitic acid, homovanillic acid, citric acid, uracil, fumaric acid, oleic acid, and azelaic acid, are determined, compared to at least six reference organic acids, and the presence or level of kidney disease in the subject is identified where the at least six organic acid levels in the subject are decreased when compared to the at least six reference organic acid levels;
(f) at least seven organic acids selected from the group consisting of glycolic acid, 3-OH isobutyric acid, 3-OH isovaleric acid, aconitic acid, homovanillic acid, citric acid, uracil, fumaric acid, oleic acid, and azelaic acid, are determined, compared to at least seven reference organic acids, and the presence or level of kidney disease in the subject is identified where the at least seven organic acid levels in the subject are decreased when compared to the at least seven reference organic acid levels;
(g) at least eight organic acids selected from the group consisting of glycolic acid, 3-OH isobutyric acid, 3-OH isovaleric acid, aconitic acid, homovanillic acid, citric acid, uracil, fumaric acid, oleic acid, and azelaic acid, are determined, compared to at least eight reference organic acids, and the presence or level of kidney disease in the subject is identified where the at least eight organic acid levels in the subject are decreased when compared to the at least eight reference organic acid levels;
(h) at least nine organic acids selected from the group consisting of glycolic acid, 3-OH isobutyric acid, 3-OH isovaleric acid, aconitic acid, homovanillic acid, citric acid, uracil, fumaric acid, oleic acid, and azelaic acid, are determined, compared to at least nine reference organic acids, and the presence or level of kidney disease in the subject is identified where the at least nine organic acid levels in the subject are decreased when compared to the at least nine reference organic acid levels; or
(i) at least ten organic acids selected from the group consisting of glycolic acid, 3-OH isobutyric acid, 3-OH isovaleric acid, aconitic acid, homovanillic acid, citric acid, uracil, fumaric acid, oleic acid, and azelaic acid, are determined, compared to at least ten reference organic acids, and the presence or level of kidney disease in the subject is identified where the at least ten organic acid levels in the subject are decreased when compared to the at least ten reference organic acid levels.

3. The method of claim 1, further comprising determining the level of:
(a) 5-oxoproline in a sample obtained from the subject, comparing the level to the level to a reference level of 5-oxoproline, and identifying the presence or level of kidney disease in the subject where the level of 5-oxoproline in the subject is increased when compared to the reference 5-oxoproline level; or
(b) citrate in a sample obtained from the subject, comparing the level to the level to a reference level of citrate, and identifying the presence or level of kidney disease in the subject where the level of citrate in the subject is decreased when compared to the reference citrate level.

4. The method of any of claim 1, wherein the subject has diabetes.

5. The method of claim 1, wherein the subject has diabetic kidney disease.

6. The method of claim 1, wherein:
(a) the reference level of the organic acid, the 5-oxoproline, or the citrate is determined from a sample obtained from a healthy patient;
(b) the organic acid is selected from the group consisting of glycolic acid, 3-OH isobutyric acid, 3-OH isovaleric acid, aconitic acid, homovanillic acid, citric acid, and uracil;
(c) the reference level of the organic acid, the 5-oxoproline, or the citrate is determined from a sample obtained from the subject at an earlier time;
(d) the reference level of the organic acid, the 5-oxoproline, or the citrate is determined from an analysis of samples obtained from more than one healthy patient;
(e) the level of the organic acid or acids is decreased at least 1.5 fold compared to the reference level;
(f) the level of the organic acid or acids is decreased at least 2 fold compared to the reference level;
(g) the level of 5-oxoproline is increased compared to the reference level; or
(h) the level of citrate is increased compared to the reference level.

7. The method of claim 1, wherein the subject has not been diagnosed with diabetes, or, the subject has kidney disease.

8. The method of claim 1, wherein the level of the organic acid, the 5-oxoproline, or the citrate is determined:
(a) using a gas chromatography;
(b) using a mass spectrometry; or
(c) from a biological sample from the subject.

9. The method of claim 1, wherein the sample contains urine, or a urine fraction, or blood, or a blood fraction.

10. A method of determining the progression of kidney disease over time in a subject diagnosed with kidney disease, comprising
a) determining the level of at least one organic acid selected from the group consisting of glycolic acid, 3-OH isobutyric acid, 3-OH isovaleric acid, aconitic acid, homovanillic acid, citric acid, uracil, fumaric acid, oleic acid and azelaic acid, in a sample obtained from the subject;
b) comparing the level of the at least one organic acid to the level of the at least one organic acid determined in a sample obtained from the subject at an earlier time point; or
c) determining that the kidney disease has progressed in the subject where the at least one organic acid level in the subject is decreased when compared to the level determined in the sample obtained from the subject at the earlier time point.

11. The method of claim 10, further comprising:
(a) determining the level of at least two organic acids selected from the group consisting of glycolic acid, 3-OH isobutyric acid, 3-OH isovaleric acid, aconitic acid, homovanillic acid, citric acid, uracil, fumaric acid, oleic acid and azelaic acid, are determined,
comparing the level of the at least two organic acids to the level of the at least two organic acids determined in a sample obtained from the subject at an earlier time point, and
determining that the kidney disease has progressed in the subject where the at least two organic acid levels in the subject are decreased when compared to the levels determined in the sample obtained from the subject at the earlier time point; or
(b) determining the level of 5-oxoproline in a sample obtained from the subject,
comparing the level to the level determined in a sample obtained from the subject at an earlier time point, and
determining that kidney disease has progressed in the subject where the level of 5-oxoproline in the subject is increased when compared to the 5-oxoproline level in the sample obtained from the subject at the earlier time point; or
(c) determining the level of citrate in a sample obtained from the subject,
comparing the level to the level determined in a sample obtained from the subject at an earlier time point, and
determining that kidney disease has progressed in the subject where the level of citrate in the subject is increased when compared to the citrate level in the sample obtained from the subject at the earlier time point.

12. The method of claim 10, wherein:
(a) the subject has diabetes or the subject has diabetic kidney disease, or the subject has not been diagnosed with diabetes;
(b) the level of the organic acid or acids is decreased at least 1.5 fold compared to the level in the sample obtained from the subject at the earlier time point;
(c) the level of the organic acid or acids is decreased at least 2 fold compared to the level in the sample obtained from the subject at the earlier time point;
(d) the level of the organic acid, the 5-oxoproline, or the citrate is determined using: gas chromatography or mass spectrometry, or the level of the organic acid is determined from a biological sample from the subject; or
(e) the sample contains urine or a urine fraction, or blood or a blood fraction.

13. A method of identifying the presence or level of joint involvement in a subject having obesity, diabetes, or chronic kidney disease, comprising
a. determining the level of at least one metabolite selected from the group consisting of 3-methyl adipic acid, 2-methyl acetoacetate, 3-methyl crotonyl glycine, 3-hydroxy propionate, 2-ethyl 3-OH propionate, and tiglylglycine in a sample obtained from the subject;
b. comparing the level of the at least one metabolite with a reference level of the at least one metabolite, wherein
i. the reference level has been determined from at least one sample collected from the same subject at a different time period; or
ii. the reference level has been determined from a sample or samples collected from one or more other subjects; and
c. identifying the presence or level of joint involvement in the subject where the at least one metabolite level in the subject is decreased when compared to the reference level of the at least one metabolite.
